(19) United States
(12) Patent Application Publication
(10) Pub. No.: US 2004/0038369 A1
(43) Pub. Date: Feb. 26, 2004

(54) Airway-specific trypsin-like enzymes and method of using the same

(76) Inventors: Hiroshi Eguchi, Tokyo (JP); Manabu Chokki, Tokyo (JP); Satoshi Yamamura, Tokyo (JP); Reiko Mita, Tokyo (JP); Tsukio Masegi, Tokyo (JP)

Correspondence Address: SUGHRUE MION, PLLC, 2100 PENNSYLVANIA AVENUE, N.W., WASHINGTON, DC, 20037, US

(21) Appl. No.: 10/362,881

(22) PCT Filed: Aug. 28, 2001
(86) PCT No.: PCT/JP01/07349

(30) Foreign Application Priority Data
Aug. 28, 2000 (JP) 2000-257104

(51) Int. Cl. G01N 33/53; C12N 9/76; C07K 16/40

(52) U.S. Cl. 435/213; 435/7.1; 530/388.26

(57) ABSTRACT

The subjects of the present invention are to provide a method of screening a compound or a polypeptide which inhibits the activity of AST, or inhibits PAR activation, mucus production promotion, cell proliferation, intracellular calcium influx or EGFR pathway activation due to AST, and further to provide a method of assaying AST in vivo and in biological cells or samples.

The present invention further includes the following inventions. ASTs whose each is protein comprising the whole amino acid sequence represented by SEQ ID NO: 1 or 2 or a part thereof or a mammalian AST protein having a 66% or more homology with the amino acid sequence represented by SEQ ID NO: 1 and in whose each a propeptide moiety is bound to a trypsin-like protein moiety via a disulfide bond. Nucleic acids encoding the same. Antibodies binding to the same. A method for assaying AST by using these antibodies. Further a method of assaying the inhibitory activity of a compound or a polypeptide to be assayed against AST or PAR activation, mucus production promotion, cell proliferation, intracellular calcium influx or EGFR pathway activation due to the AST.

### TECHNICAL FIELD

The present invention relates to airway-specific trypsin-like enzyme proteins whose structures were newly elucidated. Further, the present invention relates to a method for detecting the inhibitory activity of a compound or a polypeptide (including proteins and antibodies), using a system for detecting or assaying the mucus secretion-promoting action, inflammation-eliciting action, intracellular calcium inflow-eliciting action and protease activated receptor-activating action of said enzyme.

### BACKGROUND ART

In recent years, a human airway trypsin-like enzyme (hereinafter referred to as “airway-specific trypsin-like enzyme” or “AST” (Airway Specific Trypsin-like Protease)) has been purified from the sputum of a human chronic airway inflammation patient (JP-A 7-067640 (hereinafter, JP-A means “Japanese Unexamined Patent Publication”), Yasuoka S. et al., Am. J. Respir. Cell Mol. Biol., 16: p300-308, 1997), its amino acid sequence and cDNA sequence have been elucidated (JP-A 8-89246, U.S. Pat. No. 5,804,410, EP-699763, Yamaoka K. et al., J. Biol. Chem., 273 (19); 11895-11901, 1998).

Several activities of the enzyme have been examined in vitro. It is estimated that the enzyme may participate in the disease states of airway inflammation, because the enzyme participates in mucous ciliary movements and further has an action to reinforce the production of cytokines, such as IL-8, GM-CSF, from human bronchial epithelial cell strains (Toshiko Terao, et al., 1998 The Japanese Respiratory Society). Further, since the enzyme has enzymatic activities such as a fibrinogen-degrading activity (Yoshinaga S. et al., J. Med. Invest., 45: 77-86, 1998), and a plasminogen activator (prourokinase)-activating action (Sumiko Yoshinaga, et al., 1998 Conference on Proteases and Inhibitors in Pathophysiology and Therapeutics), it is estimated that the enzyme may anti-inflammatorily act through the formation of fibrin on the basal surface of an airway, modify the morbit state of a chronic airway patient, and further participate in metastasis and the like.

On the other hand, a protease-activated receptor (Protease Activated Receptor, hereinafter referred to as “PAR”) defined as a receptor whose signal transduction is promoted by the activity of the protease is a receptor mediating the signals by the protease, and a thrombin receptor (human PAR-1) existing in human platelets has been cloned in 1991 (Vu U K -H et al., Cell, 64: 1057-1068, 1991). Subsequently, mouse PAR-1 (Coughlin, S. R., Unpublished, Accession No. L03529, 1992), rat PAR-1 (Runge M. S. et al., J. Biol. Chem., 267: 16975-16979, 1992), mouse PAR-2 (Nystedt S. et al., Proc Natl Acad Sci USA, 91: 9208-9212, 1994), human PAR-2 (Bohm S. K., Biochem. J., 314: 1009-1016, 1996), rat PAR-2 (Saifeddine M. et al., Br. J. Pharmacol., 118(3): 521-530, 1996), human and mouse PAR-3 (Ishihara H. et al., Nature, 386: 502-506, 1997), mouse PAR-4 (Kahn M. L. et al., Nature, 394: 690-694, 1998), human PAR-4 (Xu W -F et al., Proc Natl Acad Sci USA, 95: 6642-6646, 1998), and the like, have been cloned. Many reports that PAR-1 and PAR-2 participate in various pathologic states such as inflammation have been made (Dery O. et al., Am. J. Physiol., 274: C1429-C1452, 1998).

In relation to the activation of the PAR-2, it has been reported that trypsin (Nystedt S. et al., Proc Natl Acad Sci USA, 91, 9208-9212, 1994) and tryptase (Schwartz, L. B. et al., J. Immunol., 126: 1290-1294, 1981) activate the PAR-2 (Molino M. et al., J. Biol. Chem., 272: 4043-4049, 1997).

There are a report that the PAR-2 participates in the regulation of pancreatic secretion in pancreas (Bohm S. K., Biochem. J., 314: 1009-1016, 1996), the activation of ion channels and the like (Nguyen T. D. et al., J. Clin. Invest., 103: 261-269, 1999), and a report that the PAR-2 further participates in the regulation of duodenal movements and the like (Kawabata A. et al., Br. J. Pharmacol., 128(4): 865-872, 1999).

On the other hand, reports have been made that the tryptase is released from mast cells, exhibits an enzymatic activity only as its tetramer (Schwartz, L. B. et al., J. Biol. Chem., 256: 11939-11943, 1981), and participates in skin inflammation (Steinhoff M. et al., Exp. Dermatol., 8: 282-294, 1999) and pulmonary inflammation (Am. J. Physiol. Lung Cell. Mol. Physiol., 278: L193-L201, 2000).

However, there are many still unknown points related to the actions of the tryptase and the activation of PAR2, and other PAR2-activating enzymes are entirely not elucidated. Further, there is a report suggesting the existence of the above-mentioned trypsinogen in the airway as an enzyme for activating PAR2 in the airway. However, the activity and quantity of the trypsinogen are completely not known, and only the existence of a protein by immunohistochemistry has been shown (Cocks T. M. et al., Nature, 398: 156-160, 1999). It has still more not reported that an airway-specific enzyme activating PAR2 exists.

On the one hand, in chronic inflammatory airway diseases, the exacerbation of the pathologic states due to the hypersecretion of mucous is cited as a pathologic state becoming a problem together with continuous inflammation (Jeffery, P. K. et al., Am. J. Respir. Crit. Dare. Med., 150: S6-13, 1994). The effects of macrolide drugs, such as erythromycin, against DPB (diffuse panbronchiolitis) belonging to the above-described diseases are known (Nagai, H. et al., Respiration, 58(3-4): 145-149), but the macrolide drugs are preferably not used in Europe, USA, and the like, because of being antibiotics.

Further, various theories related to target molecules mainly causing excessive mucus production promotion and secretion cell proliferation have been reported (Christian, P. et al., J. Clin. Invest., 85: 682-689, 1990), but an effective therapeutic drug does still not exist among drugs having been developed on the basis of the theories. Thereby, a drug effective for controlling the excessive mucus secretive production promotion is globally desired for chronic airway inflammatory diseases represented by COPD (Chronic Obstructive Pulmonary Disease).

### DISCLOSURE OF INVENTION

The problem of the present invention is to obtain an active AST having a true structure.

The problem of the present invention is also to construct a method for screening an agent for inhibiting the mucus production-promoting action, EGFR (Epidermal Growth Factor Receptor) pathway-activating action, inflammation-promoting action (due to cytokine production promotion or the like), intracellular calcium influx-eliciting action, and a PAR (Protease-Activated Receptor, defined as a receptor that signal tranduction is caused by the proteolytic activity)-activating action of the active AST having the true structure, and further an evaluation system for detecting the inhibitory activity.

Further, the problem of the present invention is to provide a compound or polypeptide (including proteins and antibodies) inhibiting the activity of the AST having said structure, or an evaluation system for the medicinal efficacies of the compound or the polypeptide inhibiting the mucus production-promoting action, EGFR pathway-activating action, inflammation-promoting action, intracellular calcium influx-eliciting action, and PAR-activating action of the AST having said structure.

The problem of the present invention is also to obtain an antibody binding to the enzyme having said structure and to construct an assay system, thereby providing a means for diagnosing the disorder of a secretion system, an inflammatory disease, the disorder of a congealing fibrinogenolysis system, a cancer and the like.

Namely, it can be expected that the compound or polypeptide inhibiting the activities of the AST obtained by using the method of the present invention, or the compound or polypeptide inhibiting the mucus production-promoting action, EGFR pathway-activating action, inflammation-promoting action, intracellular calcium influx-eliciting action, and PAR-activating action of the AST, is used for inhibiting or modifying the physiological actions of the AST, such as a secretion-promoting or mucus production-promoting action for the secretory cells, a congealing fibrinogenolysis system action, an airway remodeling action, an airway inflammation action, actions for the proliferation and suppression of fibroblasts•epitheliocytes•ciliated cells•smooth muscle cells•goblet cells, actions related to the proliferation and metastasis of cancer, actions for mucociliary movements, and an anti-virus infection action, and for improving and treating pathologic states.

Further, since the AST activates the PAR, it can be expected that the compound or polypeptide is used for inhibiting or modifying the physiological actions of the PAR, such as secretion-promoting actions (the mucus production-promoting actions of mucous gland cells, serous gland cells, and goblet cells, secretion-promoting actions for nerve cells•neuroendocrine cells, a chloride ion secretion-promoting action in airway epithelial cells, and the like) for secretory cells, the relaxation actions of blood vessels, airways, intestinal tracts, and the like via epithelial cells or endothelial cells, a smooth muscle-contracting action, the inflammatory cytokine production-inducing and reinforcing actions of fibroblasts, epithelial cells and the like, actions for the proliferation and suppression of cells such as fibroblasts•epithelial cells (ciliated cells, goblet cells, basal cells)•smooth muscle cells•secretory gland cells, and hypersensitiveness-reinforcing action for nerve cells, and for improving and treating pathologic states.

Furthermore, since the AST activates the EGFR pathway, it can be expected that the compound or polypeptide is used for inhibiting or modifying physiological actions caused by the activation of the EGFR, such as secretion product production-promoting actions and cytokine production-promoting actions for secretory cells (mucous gland cells, serous gland cells, goblet cells, squamous cells, cancer cells, and the like), inflammatory cytokine production-inducing and reinforcing actions for various EGFR pathway-having cells such as fibroblasts, endothelial cells, epithelial cells, and smooth muscle cells, and cell-proliferating actions for various EGFR pathway-having cells such as fibroblasts, endothelial cells, epithelial cells, ciliated cells, goblet cells, basal cells, squamous cells, cancer cells, smooth muscle cells, and secretory gland cells, and for improving and treating pathologic states.

From the direct and indirect actions on the AST, the compound or polypeptide can be used as a diagnostic drug or for an evaluation system for screening new drugs for chronic airway inflammation (chronic obstructive pulmonary diseases (including chronic bronchitis, pulmonary emphysema, diffuse pan-bronchiolitis, and bronchiectasis), bronchitic asthma), sinobronchitis syndrome, pulmonary fibrosis, respiratory anaphilaxia, pulmonary hypertension, diseases caused by congealing fibrinogenolysis system disorders, broadly-defined airway tissue cancer, arthritis, and skin inflammatory diseases.

The inventors of the present invention have took the above-described conditions into considerations and intensively made researches related to a method for purifying the AST. As the result, a method for efficiently purifying the active AST from sputum or recombinant insect cells has been established. Consequently, it has been possible to obtain the AST in a larger amount. The obtained purified protein has been used to determine its amino acid sequence. In consequence, it has clarified for the first time that the structure of the purified active AST has a protein structure in which a propeptide moiety is linked to a trypsin-like protein moiety via a disulfide bond, in addition to the already reported amino acid structure similar to that of a trypsin-like protein.

Further, it has been found that the enzyme is specifically expressed and located in large amounts in bronchial epithelium and glandulae bronchiales, especially ciliated epithelial cells, basal cells. It has also been found that the AST having said structure has an ability to activate the PAR. In addition, it has been found that the AST promotes the production of mucus in a mucus-secreting ability-having cell strain originated from a respiratory organ. The inventors of the present invention have furthermore made researches on the basis of the findings and consequently reached the present invention.

The inventors of the present invention have prepared a recombinant baculovirus vector on the basis of the cDNA sequence of HAST (namely, human AST) in insect cells (JP-A 8-89246, U.S. Pat. No. 5,804,410, EP-699763, and Yamaoka K. et al., J. Biol. Chem., 273 (19): 11895-11901, 1998), and expressed the recombinant protein. The active protein has been purified from the recombinant protein by the purification method shown in Example 1, and the primary structure of the active protein has been determined. Consequently, it has been found that the active protein contains a protein having a structure in which a propeptide partial moiety sequence comprising an amino acid sequence between methionine (Met) at the 1^{st} position and arginine (Arg) at the 186^{th} position (concretely, a propeptide moiety started from asparagine (Asn) at the 145^{th} position or aspartic acid (Asp) at the 162^{nd} position) is bound to a trypsin-like protein moiety via a disulfide bond, in addition to the already reported trypsin-like structure protein (JP-A 7-067640, S. Yasuoka et al., Am. J. Respir. Cell Mol. Biol., 16: p300-308, 1997, a trypsin-like protein moiety starting from isoleucine (Ile) at the 187^{th} position). And, it has been discovered that the protein having the structure mentioned above is the main component of the active AST.

Further, natural HAST has been purified from sputum, and the amino acid sequence of the natural HAST has similarly been determined. Consequently, it has again been found that there is a protein having a structure in which a propeptide partial moiety sequence (concretely, a propeptide moiety starting from isoleucine (Ile) at the 173^{rd} position) is bound to a trypsin-like protein moiety starting from isoleucine (Ile) at the 187^{th} position via a disulfide bond. And, it has been found that the protein having the structure is the main component of the active AST.

Meanwhile, the HAST has been considered to specifically exist in human being, and has been named HAT (Human Airway Trypsin-like Protease) (JP-A 7-067640, S. Yasuoka et al., Am. J. Respir. Cell Mol. Biol., 16: p300-308, 1997). However, the inventors of the present invention have carried out a PCR (Polymerase Chain Reaction) using a degenerate primer prepared on the basis of the homology of several serine proteases and further using a cDNA prepared from a mouse airway as a template, and determined the sequence of the obtained DNA fragment. Consequently, it has been fount for the first time that a HAST-like protein exists also in an animal, such as mouse, not carrying out standing bipedal locomotion. In addition, the full length cDNA encoding the AST-like protein of the mouse has been obtained by executing 5′-RACE (Rapid Amplification of cDNA Ends), 3′-RACE. The inventors of the present invention have examined the tissue specificity of the gene expression, and also observed an airway-specific expression in the mouse. Further, the obtained mouse AST gene has been expressed in insect cells as a recombinant, and the primary structure of the protein has been determined. In consequence, it has been clarified that similarly to the human AST (HAST), the mouse AST is also an enzyme having a structure in which the partial sequence of a propeptide comprising an amino acid sequence between methionine (Met) at the 1^{st} position and arginine (Arg) at the 186^{th} position is bound to a trypsin-like protein moiety starting from isoleucine (Ile) at the 187^{th} position via a disulfide bond. By the similar method, the cloning of hamster AST and the cloning of guinea pig AST have also been succeeded.

Additionally, by methods similar to the above-described methods, it has been found for the first time that HAST-like proteins exist also in higher mammals such as monkey, pig, dog, rabbit, and bovine. Concretely, a degenerate primer designed on the basis of cDNA sequences of human and rodent ASTs has been prepared, and a PCR using a cDNA synthesized from a total RNA extracted from the airway of a higher mammal as a template has been carried out to amplify the partial cDNA sequence of the AST of the higher mammal, followed by determining the DNA sequence. A primer for RACE has been prepared on the basis of the determined DNA sequence, and 5′-RACE, 3′-RACE are performed to obtain the full length cDNA encoding the HAST-like protein. The mammalian AST gene thus obtained has transiently been expressed in human cultured cells, and it has consequently been found that a trypsin-like activity similar to that of the human AST (HAST) can be expressed. Further, a simian AST gene has been expressed in insect cells as a recombinant, and the primary structure of the protein has been determined. Consequently, the N-terminal amino acid sequence of a trypsin-like protein moiety starting from IIGG and the partial amino acid sequence of a propeptide starting from DQAA have been detected in equimolar amounts, respectively. And, it has been clarified that, similarly to the human AST, the simian AST is also an enzyme having a structure in which a propeptide partial sequence comprising an amino acid sequence between methionine (Met) at the 1^{st} position and arginine (Arg) at the 186^{th} position is bound to a trypsin-like protein moiety starting from isoleucine (Ile) at the 187^{th} position via a disulfide bond.

The above-described discovery of the existence of the animal AST enables the evaluation of the expression of the animal AST at a gene level or at a protein level, and the evaluation in the expression and activity of the animal AST in an animal disease model is greatly useful for clarifying the participation of the AST in said pathological state. Additionally, the participation of the AST in a pathologic model animal can be examined by inhibiting the expression of the AST with an anti-sense oligonucleotide or transducing an expression vector to increase the expression of the AST. Further provided is information which is important on the evaluation of the medicinal efficacies of an AST inhibitor or an inhibitory polypeptide (including proteins and antibodies) in an animal model and also important on the examination and evaluation of a species difference between animals.

Next, a recombinant AST having the above-described structure has been allowed to act on a recombinant PAR-expressing insect cell, and the activation of the PAR has been evaluated, while using calcium influx as an indicator. Consequently, it has been found for the first time that AST activates PAR and the calcium influx is caused in the insect cell in which PAR1 and PAR2 are expressed.

Further, they have been found for the first time that the AST activates the PAR in normal human airway epithelial cells and airway epithelial cell strain to cause calcium influx and that the AST having said structure causes IL-8 production promotion and cell proliferation with a signal transduction system mediating the PAR, and it has been clarified that the AST modifies the morbid state of an airway disease with the signal transduction system mediating the PAR. And, a screening system based on the enzymatic activity or PAR activation action of a recombinant AST protein having said structure has been constructed on the basis of the results.

In addition, it has been found for the first time that, in a human mucus-secreting cell strain, the AST having said structure promotes mucus production, causes intracellular calcium influx, activates an EGFR pathway, ant the like to cause IL-8 production promotion and cell proliferation. And it has been clarified that, in a chronic airway disease in which the mucus production is promoted, the AST promotes the mucus production, causes the intracellular calcium influx, activates the PAR, the EGFR pathway, and the like to cause the IL-8 production promotion and the cell proliferation, thus modifying the pathologic state.

Furthermore, it has thus been found that a screening system can be constructed on the basis of the enzymatic activity or mucus production-promoting activity, PAR-activating action, intracellular calcium influx-eliciting action, EGFR pathway-activating action and IL-8 production-reinforcing action of the recombinant AST protein having the above-described structure, and the present invention has thus been completed.

Namely, the present invention is the AST having the structure in which the propeptide moiety is bound to the trypsin-like protein moiety via the disulfide bond.

Further, the present invention is the inhibitor or inhibitory polypeptide-screening system using the mucus production-promoting action, PAR-activating action, intracellular calcium influx-eliciting action, EGFR pathway-activating action and IL-8 production-reinforcing action or cell proliferation-promoting action of the enzyme having said structure as an indicator.

Further, the present invention includes animal cells, such as insect cells, producing the AST having said structure.

In addition, the present invention is an antibody which binds to the enzyme having said structure and comprises a monoclonal antibody or polyclonal antibody having a bonding property enabling the detection of said enzyme in vivo by an enzyme immunoassay, or a monoclonal antibody or polyclonal antibody having a bonding property enabling the detection of said enzyme in a tissue or in a cell by an immune tissue dyeing method, or a monoclonal antibody or polyclonal antibody capable of inhibiting said enzyme activity.

Furthermore, the present invention includes animal cells, such as hybridoma cells, producing the monoclonal antibody.

The present invention will more concretely be described as follows.

(1). An AST which is a protein comprising the whole amino acid sequence represented by SEQ ID NO: 1 or a part thereof and has a structure in which a propeptide moiety comprising the whole amino acid sequence from Met at the 1^{st} position to Arg at the 186^{th} position or a part thereof is bound to a trypsin-like protein moiety comprising a sequence of 232 amino acids from Ile at the 187^{th} position to Ile at the 418^{th} position via a disulfide bond.

(2). A mammalian AST which is a mammalian AST counterpart protein having a 66% or more homology with the amino acid sequence represented by SEQ ID NO: 1 and has a structure in which a moiety corresponding to said propeptide moiety is bound to a moiety corresponding to said trypsin-like protein moiety via a disulfide bond.

(3). A mammalian AST which is a protein comprising the whole amino acid sequence represented by SEQ ID NO: 2, 27, 29, 31, 33, 35, 37 or 39 or a part thereof and has a structure in which a propeptide moiety comprising the whole amino acid sequence from Met at the 1^{st} position to Arg at the 185^{th} or 186^{th} position or a part thereof is bound to a polypeptide moiety comprising a sequence of 232 amino acids from Ile at the 186^{th} or 187^{th} position to Ile or Val at the 417^{th} or 418^{th} position via a disulfide bond.

(Hereinafter, the AST having either of the above-described structures (1) to (3) is simply described as “airway-specific trypsin-like enzyme” or “AST”).

(4). An antibody specifically binding to AST.

(5). A monoclonal antibody inhibiting human AST activity, or inhibiting its activation.

(6). A method for detecting or assaying AST with the above-described antibody.

(7). A method for detecting the AST-inhibiting activity of a compound or a polypeptide to be assayed, by mixing an enzyme substrate with the compound or the polypeptide to be assayed, incubating the mixture and the AST or a cell expressing said enzyme or a tissue expressing said enzyme under proper conditions to react with said enzyme substrate, and then assaying the reaction product.

(8). A method for detecting the inhibitory activity of a compound or a polypeptide to be assayed against PAR activation due to AST, by mixing the AST or a cell expressing said enzyme or a tissue expressing said enzyme, the compound or the polypeptide to be assayed, and further a PAR-expressing cell, and then using the PAR activation as an indicator.

(9). A method for detecting the inhibitory activity of a compound or a polypeptide to be assayed against intracellular calcium influx due to AST, by mixing the AST or a cell expressing said enzyme or a tissue expressing said enzyme, the compound or the polypeptide to be assayed, and further a cell having an intracellular calcium influx ability, and then using the intracellular calcium influx as an indicator.

(10). A method for detecting the inhibitory activity of a compound or a polypeptide to be assayed against a mucus production-promoting action due to AST, by mixing the AST or a cell expressing said enzyme or a tissue expressing said enzyme, the compound or the polypeptide to be assayed, and further a cell having a mucus-producing ability, and then using the secretion product as an indicator.

(11). A method for detecting the inhibitory activity of a compound or a polypeptide to be assayed against an EGFR-L (EGFR-ligand)-releasing ability due to AST, by mixing the AST or a cell expressing said enzyme or a tissue expressing said enzyme, the compound or the polypeptide to be assayed, and further a cell having an EGFR-L, and then using the release quantity of the EGFR-L as an indicator.

(12). A method for detecting the inhibitory activity of a compound or a polypeptide to be assayed against an EGFR signal transduction system-activating action due to AST, by mixing the AST or a cell expressing said enzyme or a tissue expressing said enzyme, the compound or the polypeptide to be assayed, and further a cell having an EGFR signal transduction pathway, and then using the EGFR pathway signal transduction as an indicator.

(13). The coding domain of a natural mammalian AST gene.

(14). A mRNA or cDNA originated from a mammalian AST gene. ([for example], SEQ ID NO: 3, 4, 5, 6, 26, 28, 30, 32, 34, 36, 38).

(15). A mammalian AST.

(16). An expression vector prepared with the nucleic acid of the above-described (13) or (14). A cell transfected or transformed with the expression vector. A method for purifying an AST from the cell and the purified AST.

(17). A method for detecting the expression quantity of an AST gene corresponding to the above-described nucleic acids with the whole or partial information of the nucleic acids.

(18). A method for screening a substance inhibiting the activity of AST or inhibiting the activation of said enzyme or a method for judging a therapeutic effect thereof, comprising administering a compound or a polypeptide to be assayed to a mammal having a disease characterized by the activation excess or up-regulate of the AST.

(19). A method for screening a substance inhibiting mucus production-promoting action, inflammation-promoting action (due to cytokine production promotion or the like), intracellular calcium influx action, PAR-activating action, and EGFR pathway-activating action due to AST or a method for judging its therapeutic effect, comprising administering a compound or a polypeptide to be assayed to a mammal having a disease characterized by the mucus production promotion, inflammation promotion, intracellular calcium influx promotion, EGFR pathway activation, and PAR activation due to the AST.

(20). A method for judging a therapeutic effect due to the inhibition of the activity of AST or the inhibition of its activation, by administering a drug containing the above-described anti-sense oligonucleotide to a mammal having a disease characterized by the activation excess or up-regulate of the AST.

### BRIEF DESCRIPTION OF DRAWINGS

FIG. 1 shows the results of AB-PAS staining in a mucus-secreting cell strain NCI-H292 by the addition of EGF-L.

FIG. 2 shows the results of AB-PAS staining in a mucus-secreting cell strain NCI-H292 by the addition of the HAST of the present invention or EGF-L.

FIG. 3 shows the results of AB-PAS staining in a mucus-secreting cell strain NCI-H292 by the addition of the HAST of the present invention and a serine protease inhibitor.

FIG. 4 shows the results of AB-PAS staining in a mucus-secreting cell strain NCI-H292 by the addition of the HAST of the present invention or thermally treated HAST.

FIG. 5 shows the assay results of the mRNA quantities of Muc5AC, when a protease inhibitor was simultaneously added to a mucus-secreting cell strain NCI-H292 to which the HAST of the present invention was added.

FIG. 6 shows the assay results of the mRNA quantities of Muc5AC, when an EGFR neutralizing antibody or a protease inhibitor was simultaneously added to a mucus-secreting cell strain NCI-H292 to which the HAST of the present invention was added.

FIG. 7 shows the assay results of the mRNA quantities of Muc5AC in a mucus-secreting cell strain NCI-H292 to which the AST of the present invention, trypsin, tryptase, or elastase was added.

FIG. 8 shows assay results that the IL-8 release promotion from an airway epithelial cell strain BEAS-2B by the addition of AST of the present invention is inhibited with protease inhibitors.

FIG. 9 shows that the IL-8 release promotion from an airway epithelial cell strain BEAS-2B by the addition of AST of the present invention occurs at a transcription level.

FIG. 10 shows that the AST of the present invention promotes the tyrosine phosphorylation of EGFR in mucus-secreting cell strain NCI-H292.

FIG. 11 is a figure showing the homology of the amino acid sequence of human AST and mouse AST.

FIG. 12 shows calcium influx in PAR-expressing insect cells due to the AST of the present invention.

FIG. 13 shows calcium influx due to the AST of the present invention with the normal human airway epithelial cells.

FIG. 14 shows calcium influx due to the AST of the present invention with the airway epithelial cell strain.

FIG. 15 shows the evaluation results of the inhibitory activities of the anti-AST antibodies of the present invention.

FIG. 16 shows the standard curve of an assay system using the anti-AST polyclonal antibody of the present invention against a recombinant AST.

FIG. 17 shows the assay results of AST in sputum by an assay system using the anti-AST polyclonal antibody of the present invention.

FIG. 18 shows results obtained by examining the liquid phase antigenic reactivity of the anti-AST monoclonal antibody and anti-AST polyclonal antibody of the present invention with the recombinant AST.

FIG. 19 shows the standard curve of an assay system using the anti-AST monoclonal antibody and anti-AST polyclonal antibody of the present invention against the recombinant AST.

### BEST MODE FOR CARRYING OUT THE INVENTION

In the airway-specific trypsin-like enzyme of the present invention comprising the whole amino acid sequence represented by SEQ ID NO: 1 or a part thereof, it is preferable that the propeptide moiety is bound to the trypsin-like protein moiety via the disulfide bond of Cys at the 173^{rd} position to Cys at the 292^{nd} position. In the airway-specific trypsin-like enzyme of the present invention comprising the whole amino acid sequence represented by SEQ ID NO: 2 or a part thereof, it is preferable that a moiety corresponding to the propeptide moiety is bound to a moiety corresponding to the trypsin-like protein moiety via the disulfide bond of Cys at the 172^{nd} position to Cys at the 291^{st} position. In the mammalian airway-specific trypsin-like enzyme of the present invention having the 66% or more homology with the amino acid sequence represented by SEQ ID NO: 1, it is preferable that a moiety corresponding to the propeptide moiety is bound to a moiety corresponding to the trypsin-like protein moiety via the disulfide bond of a cysteine moiety corresponding to Cys at the 173^{rd} position to a cysteine moiety corresponding to Cys at the 292^{nd} position in the AST represented by SEQ ID NO: 1.

In a case of the human AST, as the propeptide moiety is preferable a propeptide moiety whose N-terminal (amino terminal) amino acid is an amino acid between Met at the 1^{st} position and Ile at the 170^{th} position, an amino acid between Asp at the 44^{th} position and Ile at the 170^{th} position, or an amino acid between Asn at the 145^{th} position and Ile at the 170^{th} position. A propeptide moiety ranged from either of Asn at the 145^{th} position, Asp at the 162^{nd} position and Ile at the 170^{th} position to Arg. at the 186^{th} position as the C-terminal (carboxy terminal) is especially preferable. A propeptide moiety ranged from either of Asp at the 162^{nd} position and Ile at the 170^{th} position to Arg at the 186^{th} position is most preferable.

As an antibody to be specifically bound to the above-described AST of the present invention is preferably a monoclonal antibody, especially preferably a monoclonal antibody which inhibits the activity of the human AST or inhibits its activation.

An enzyme immunoassay is preferably used as an AST-detecting or assaying method using the antibody of the present invention.

The AST or AST-expressing cell used in the method for screening the AST-inhibiting activity of the compound or polypeptide to be assayed, of the present invention is generally the structure-having AST of the present invention or a cell producing said AST, preferably an AST-expressing cell or organ, tissue originated from an oral tissue, nasal tissue, paranasal sinus tissue, pharyngeal tissue, laryngeal tissue, trachea, bronchi or pulmonary tissue, a cell originated from the tissue or a cell strain thereof. The cell or cell strain originated from the tissue includes cancer cells and cancer cell strains. Especially, epithelial cells, in particular, ciliated epithelium cells or basal cells or cell strains originated from the cells are preferable. The purified AST of the present invention, having the above-described structure is most preferable for being supplied to evaluation.

In the method for screening the inhibitory activity of the compound or polypeptide to be assayed against the mucus production-promoting action due to the AST, of the present invention, the dyeing quantity of the mucus glycoprotein by an AB-PAS staining method, the release quantity of a sulfuric acid-labeled polymer, the production quantity of mucin, the mRNA expression quantity of mucin (especially Muc5AC), the promoter transcription activity of the mucin (especially Muc5AC), the ligand-producing or releasing quantity of an EGF receptor, the phosphorylated tyrosine quantity of a protein participated in an EGF receptor signal transduction pathway, or the quantity of a phosphorylated MAPK (MAP kinase) is preferably cited as the indicator of the mucus production-promoting action.

In a method for screening the inhibitory activity of the compound or polypeptide to be assayed against the EGFR pathway-activating action due to the AST, of the present invention, the cutting or release quantity of the EGFR-L (EGFR-Ligand), the assay target of the already reported EGFR signal transduction system (Wells A., Int. J. of Biochem. & Cell Biology, 31: 637-643, 1999), a phenomenon caused by the signal transduction, or the like, can be used as the indicator of the EGFR pathway activation to assay the EGFR signal transduction system activation due to the AST.

The indicator of the EGFR pathway activation includes intracellular calcium influx, a cell proliferation assay, the assay of the transcription activity of a gene (IL-8, IL-6, GM-CSF or the like) containing a transcription element such as NFkB or AP-1, the assay of a produced protein, the assay of an inositol phosphate-degrading activity in cells, the assay of prostaglandin E₂ production, the assay of thromboxane A₂, the assay of cAMP (cyclic AMP), the assay of a mucus production quantity, the production quantity of mucin, the mRNA expression quantity of mucin (especially Muc5AC), the promoter transcription activity of the mucin (especially Muc5AC), and the phosphorylated tyrosine quantity of a protein (the phosphorylated tyrosine quantity of EGFR, MAPK (MAP kinase) or the like) participated in an EGF receptor signal transduction pathway.

In a method for screening the inhibitory activity of the compound or polypeptide to be assayed against the PAR-activating action due to the AST, of the present invention, the PAR signal transduction system activation due to the AST can be assayed with the already reported assay target as the indicator of the PAR activation signal transduction.

The indicator of the PAR activation signal transduction includes an intracellular calcium influx, a cell proliferation assay, the assay of the transcription activity or produced protein of a gene (IL-8, IL-6, GM-CSF or the like) containing a transcription element such as NFkB or AP-1, the assay of an inositol phosphate-degrading activity in cells, the assay of prostaglandin E₂ production, the assay of thromboxane A₂, the assay of cAMP (cyclic AMP), a method for directly or indirectly assaying the production of NO (Nitric Oxide), the assay of calcium release or electric current with Xenopus Oocyte, the assay of a chloride ion transport quantity via a chloride channel, and the assay of a secreted mucus quantity.

Additionally, the AST-expressing cell is preferably a recombinant cell expressing said enzyme. Further, the AST-expressing cell or a tissue expressing said enzyme is preferably originated from an oral tissue, nasal tissue, paranasal sinus tissue, pharyngeal tissue, laryngeal tissue, trachea, bronchi or pulmonary tissue, or is preferably a cell originated from the tissue, an organ or the tissue, or a cell strain thereof. The cell or cell strain originated from the tissue includes cancer cells and cancer cell strains. Especially, epithelial cells, particularly, airway epithelial cells, more particularly, ciliated epithelium cells or basal cells or cell strains originated from the cells are preferable. In addition, the AST-expressing cell includes goblet cells or a cell strain originated from the goblet cells, mucous gland cells or a cell strain originated from the mucous gland cells, and serous gland cells or a cell strain originated from the serous gland cells.

The cells for evaluating the secretion include a mucus-secreting cell strain, NCI-H292, and preferably further include secretory ability-having cells, organ or tissue existing in an oral tissue, nasal tissue, paranasal sinus tissue, pharyngeal tissue, laryngeal tissue, trachea, bronchi or pulmonary tissue, preferably airway epithelial cells, particularly ciliated epithelium cells or basal cells or cell strains originated from the cells. The cells or cell strain originated from the tissue include cancer cells and cancer cell strains. In addition, the secretory ability-having cells preferably include goblet cells or a cell strain originated from the goblet cells, mucous gland cells or a cell strain originated from the mucous gland cells, and serous gland cells or a cell strain originated from the serous gland cells. NCI-H 292 cells, A549 cells and the like established as cell strains are most preferably supplied for the evaluation.

The PAR-expressing cells include PAR-expressing recombinant insect cells, and preferably further include PAR-expressing cells, organ or tissue existing in an oral tissue, nasal tissue, paranasal sinus tissue, pharyngeal tissue, laryngeal tissue, trachea, bronchi or pulmonary tissue, especially preferably airway epithelial cells, particularly ciliated epithelium cells or basal cells or cell strains originated from the cells. In addition, the PAR-expressing cells preferably include goblet cells or a cell strain originated from the goblet cells, mucous gland cells or a cell strain originated from the mucous gland cells, serous gland cells or a cell strain originated from the serous gland cells, airway smooth muscle cells, pulmonary fibroblasts, and nerve cells. BEAS-2B established as a cell strain is most preferably supplied for the evaluation.

The concrete examples of the PAR are protease-activating receptors to be activated with the AST, preferably PAR1 and PAR2 among known PAR1, PAR2, PAR3, and PAR4, most preferably PAR2 among the four PARs.

The EGFR-L-expressing cells include recombinant EGFR-L-expressing animal cells, and preferably further include cells, organs or tissues existing in an oral tissue, nasal tissue, paranasal sinus tissue, pharyngeal tissue, laryngeal tissue, trachea, bronchi or pulmonary tissue, especially preferably airway epithelial cells, particularly ciliated epithelium cells or basal cells or cell strains originated from the cells. In addition, the EGFR-L-expressing cells preferably include goblet cells or a cell strain originated from the goblet cells, mucous gland cells or a cell strain originated from the mucous gland cells, serous gland cells or a cell strain originated from the serous gland cells, airway smooth muscle cells, pulmonary fibroblasts, and nerve cells.

Concrete examples include HCI-H292, A549, and the like established as cell strains. Recombinant EGFR-L-expressing insect cells or animal cells are preferably supplied for evaluation.

The concrete example of the EGFR-L is generally EGFR-L to be released with the AST, and includes known EGF, HB-EGF, TGF-α, Amphiregulin, and Betacellulin. EGF and HB-EGF are preferable among the known substances, and EGF is most preferable.

The EGFR-expressing cells include NCI-H292 cell strain, and preferably further include cells, organs or tissues existing in an oral tissue, nasal tissue, paranasal sinus tissue, pharyngeal tissue, laryngeal tissue, trachea, bronchi or pulmonary tissue, especially preferably airway epithelial cells, particularly ciliated epithelium cells, squamous cells, basal cells, or cancer cells or cell strains originated from the cells. In addition, the EGFR-L-expressing cells preferably include goblet cells or a cell strain originated from the goblet cells, mucous gland cells or a cell strain originated from the mucous gland cells, serous gland cells or a cell strain originated from the serous gland cells, airway smooth muscle cells, pulmonary fibroblasts, and nerve cells. The EGFR-L-expressing cells are most preferably HCI-H292, A549 and the like, established as cell strains.

The concrete example of the EGFR-L is generally EGFR-L to be released with the AST, and includes known EGF, HB-EGF, TGF-α, Amphiregulin, and Betacellulin. EGF and HB-EGF are preferable among the known substances, and EGF is most preferable.

The mRNA or cDNA originated from the mammalian AST gene of the present invention includes that of mouse, such as a mRNA or cDNA encoding an AST represented by SEQ ID NO: 5 or SEQ ID NO: 6. Similarly, the mammalian AST of the present invention includes those of monkey, pig, dog, rabbit, cattle, hamster, and guinea pig, concretely a mRNA or cDNA encoding an AST represented by SEQ ID NO: 27, 29, 31, 33, 35, 37, or 39.

The present invention includes the expression vector indicating the expression of the AST of the present invention and prepared with the whole or partial information of the nucleic acid sequences; the cell transfected or transformed with the expression vector; the method for producing the AST, comprising culturing the cell and then recovering the structure-having active AST of the present invention from the culture solution or cultured cells; and the AST obtained by the production method. Further, the present invention includes the natural mammalian AST purified from the body fluid, cells or cell strains of a mammal in which the natural mammalian AST exists.

Furthermore, the present invention is a method for detecting the expression quantity of the AST gene corresponding to the nucleic acids with the whole or partial information of the nucleic acids, such as a northern hybridization method or a gene amplification method. The present invention further includes a sense primer or anti-sense primer which is used for the gene amplification method and is prepared on the information of a natural mammalian AST gene sequence, such as the information of a natural mammalian cDNA sequence.

The present invention is further a method for screening a substance inhibiting the activity of the AST or inhibiting the activation of said enzyme, comprising administering the compound or polypeptide to be assayed to a mammal having a disease characterized by the activation excess or up-regulate of the above-described AST, or a method for judging the therapeutic effect of the substance.

The disease characterized by the activation excess or up-regulate of the above-described AST includes chronic airway inflammation, chronic obstructive pulmonary diseases, chronic bronchitis, pulmonary emphysema, diffuse panbronchiolitis, bronchiectasis, asthma, sinobronchitis syndrome, fibrosis, nerve oversensitivity, diseases due to congealing fibrinogenolysis system disorders, cancer, arthritis, and skin-inflammatory diseases, and especially includes inflammatory diseases in respiratory systems and diseases whose pathologic conditions are caused by secretion disorders in oral tissues, nasal tissues, paranasal sinus tissues, pharyngeal tissues, laryngeal tissues, tracheae, bronchi, or pulmonary tissues, extracellular substrate synthesis disorders, cell amplification disorders, cell differentiation disorders, nerve system disorders, immune system disorders, mucociliary transport system disorders or congealing fibrinogenolysis system disorders.

The disease characterized by the mucus production promotion, inflammation promotion, intracellular calcium influx promotion, EGFR pathway activation, or PAR activation due to the AST includes chronic airway inflammation, chronic obstructive pulmonary diseases, chronic bronchitis, pulmonary emphysema, diffuse panbronchiolitis, bronchiectasis, asthma, sinobronchitis syndrome, fibrosis, nerve oversensitivity, diseases due to congealing fibrinogenolysis system disorders, cancer, arthritis, and skin-inflammatory diseases, and especially includes inflammatory diseases in respiratory systems and diseases whose pathologic conditions are caused by secretion disorders in oral tissues, nasal tissues, paranasal sinus tissues, pharyngeal tissues, laryngeal tissues, tracheae, bronchi, or pulmonary tissues, extracellular substrate synthesis disorders, cell amplification disorders, cell differentiation disorders, nerve system disorders, immune system disorders, mucociliary transport system disorders or congealing fibrinogenolysis system disorders.

In addition, the present invention includes a method for judging a therapeutic effect due to the inhibition of the activity or activation of the above-described AST, by administering a drug containing the anti-sense oligonucleotide to the mammal having the disease characterized by the activation excess or up-regulate of the AST, wherein the anti-sense oligonucleotide is prepared with a part of the above-described nucleic acid of the present invention, and a drug which contains the anti-sense oligonucleotide as an active ingredient and is used for treating said disease. The disease characterized by the activation excess or up-regulate of the AST concretely includes the same diseases as the above-described diseases.

In Examples 13, 14 of the present invention, the mucus production-promoting action of the AST is evaluated with the NCI-H292 cell strain, and the cell used in the system for screening the mucus production promotion inhibition of the AST inhibitors is preferably a cell having a secreting ability, more preferably a cell, organ or tissue having the secreting ability in a respiratory system. The NCI-H292 cell, A549 cell or the like established as a cell strain is most preferable to supply for the evaluation.

The secretion product used as the indicator is an organic substance or an inorganic substance, and the organic substance is preferably a glycoprotein, more preferably a mucus. Mucin which is a component of the mucus is most preferable as a secretion indicator from relations with pathologic states (Tsuda, T. et al., “Molecular Biology of Respiratory Diseases”; 87-92, 1998). Further, Muc5AC (Meerzaman, D. et al., J. Biol. Chem., 269: 12932-12939, 1994), Muc2 (Gum, J. R. Jr. et al., J. Biol. Chem., 269 (4): 2440-2446, 1994), and the like, to which attentions are paid in the pathologic states of hypersecretion such as COPD are especially preferable among the mucin.

The execution form of the assay indicator in the HAST inhibitor-screening system based on the mucus production-promoting action preferably includes (1) an enzyme immune assay using a monoclonal antibody 17Q2 (human airway mucin is used as an antigen) which is an antibody against the mucin antigen (Thomas, E. et al., Am. J. Respir. Cell Mol. Biol., 14: 104-112, 1996) or the like, the mucus-dyeing method (AB-PAS dyeing) shown in Example 13, and a method for assaying the release quantity of a sulfuric acid-labeled polymer, at the level of glycoproteins, and (2) a real time PCR method disclosed in Example 14 as a method for assaying the mRNA quantity of the mucin (preferably Muc5AC, Muc2), and a reporter gene assay system using the promoter domain of the mucin as a method for evaluating the transcription activity of the gene, at the level of genes.

In addition, since the Mucr5AC mRNA increase based on the activity of the HAST is blocked with the EGF-R neutralizing antibody as shown in Example 14, it can be estimated that the signal transduction of the EGF-R pathway can become one of the main pathways of the mucus production promotion due to the AST (Takeyama, K. et al., Proc. Natl. Acad. Sci. USA, 96: 3081-3086, 1999). The effect of the AST inhibitor can be assayed by quantifying the reaction participating in the EGF-R signal transduction pathway (Louis, M. L. et al., Current Opinion in Cell Biology, 11: 177-183, 1999). If the reactions can be quantified in the above-described related pathway, all of the reactions will be able to be used as assay indicators. For example, the phosphorylation of the tyrosine residue of the EGF-R and the phosphorylation of MAPK (Mitogen-Activated Protein Kinase) in target cells can be used as indicators in an immune assay.

The execution form of the AST inhibitor-screening system based on the mucus production-promoting action includes a mucus production-inhibiting effect assay which comprises adding the AST to a tissue, organ or cultured cell having a mucus-secreting ability, adding a compound or the like, and then assaying the quantity of the secretion product, the quantity of the mRNA, the transcription activity of said gene, or the like. Further, as described above, the inhibition effect can be judged by a method for directly assaying the content of the EGFR-L in the organ or the cell culture supernatant, a method for adding to any other cell system and then indirectly assaying the content of the EGFR-L, or the like. The EGFR-L to be assayed preferably includes EGF (Epidermal Growth Factor, Carpenter, G. et al., J. Biol. Chem., 265: 7709-7712, 1990), HB-EGF (Heparin-binding EGF-like Growth Factor, Abraham, J. A. et al., Biochem. Biophys. Res. Commun., 190: 125-133, 1993), TGF-α (Transforming Growth Factor-α, Lee, D. C. et al., Pharm. Rev., 47: 51-85, 1995), Amphiregulin (Plowman, G. D. et al., Mol. Cell. Biol., 10: 1969-1981, 1990), and Betacellulin (Shing, Y et al., Science, 259: 1604-1607, 1993) (Gerhard, R. et al., Biochim. Biophys. Acta, 1333: F179-F199, 1997). EGF, HB-EGF are especially preferable.

### EXAMPLES

The present invention will be explained in more detail hereafter with examples, but the present invention is not limited to the examples.

### Example 1

### Preparation of AST

The purification of an active natural AST from a sputum was carried out by the same method as the below-described method for purifying a recombinant AST.

The purification of a recombinant HAT was carried out by preparing a recombinant baculovirus vector on the basis of a cDNA sequence disclosed in JP-A 8-89246, U.S. Pat. No. 5,804,410, EP-699763, and Yamaoka K. et al., J. Biol. Chem., 273(19): 11895-11901, 1998), infecting insect cells with the recombinant baculovirus vector to produce the recombinant AST (rAST), and then purifying the rAST from the cell fraction of the product. Namely, the insect cells (Tn-5 cells) were allowed to grow up to a density of 5×10⁶ cells/mol in a single layer, and the culture medium was then removed. A serum-free culture medium containing the recombinant AST-expressing baculovirus was added to the grown insect cells in an MOI (Multiplicity of Infection) of 0.2 to 0.5 per cell to infect the insect cells with the recombinant AST-expressing baculovirus, and the infected insect cells were cultured for three days to express the AST. The identification of the expressed protein was carried out by a western blot method using SDS-PAGE and an anti-AST peptide antibody (Anal. Biochem., 112, 195-203, 1981).

The culture product was centrifuged (about 500×g) to separate the cells from the supernatant. The cells corresponding to 450 mL were suspended in 100 mL of M-PER (Mammalian Protein Extraction Reagent, produced by PIERCE Corp.), incubated at room temperature for 30 minutes, and further subjected to an ultrasonic crushing treatment on ice for 15 minutes to lyse the cells. The obtained lysate was centrifuged at 10,000 rpm at 4° C. for 30 minutes, and the supernatant was recovered. The recovered supernatant was dialyzed with 1 liter of 50 mM sodium acetate (pH 4)/0.01% PEG 6000 at room temperature for two hours with stirring, and then centrifuged at 10,000 rpm at 4° C. for 30 minutes to recover the supernatant.

Subsequently, the recovered sample was mixed with BSA (final concentration: 100 μg/mL), twice analyzed with 2 liters of 50 mM Tris-HCl (pH 8)/0.5M NaCl/0.01% PEG 6000, and then centrifuged at 10,000 rpm at 4° C. for 30 minutes to recover the supernatant. The recovered supernatant was put on benzamidine sepharose 6B (10 mL bed volume), washed with 100 mL of 50 mM Tris-HCl (pH 8)/0.5M NaCl/0.01% PEG 6000 in a column, further washed with 80 mL of a 10 mM sodium phosphate buffer solution (pH 7.5) in the column, and then subjected to the elution of the bound protein with 10 mM HCl (pH 2). After the elution, the absorbance (280 nm) of each fraction was assayed, and the main peak fractions were collected. The collected main peak fractions were subjected to an SDS polyacrylamide gel electrophoresis (SDS-PAGE) to detect and identify the protein having a molecular weight of about 30 kD, which was frozen and stored as the purified rAST sample.

### Example 2

### Activity Assay of AST

50 μL of an AST-containing solution was added to 1.0 mL of a 50 mM Tris-HCl buffer solution (pH 8.6) containing 100 μM of a synthetic substrate Boc-Phe-Ser-Arg-MCA (MCA: methylcoumarinamide) for trypsin and 0.01% of BSA and then incubated at 37° C. for one hour. Subsequently, 1 mL of 30% acetic acid was added, and the produced 7-amino-4-methylcoumarin (AMC) was assayed by fluorometry (fluorescent light: 460 nm, exciting light: 380 nm). The enzymatic activity was calculated. The activity for producing 1 pM of AMC for one minute was defined as one unit.

### Example 3

### Determination of Amino Acid Sequences of Recombinant AST and Natural AST

The purified protein obtained in Example 1 was subjected to SDS-PAGE under reducing and non-reducing conditions, respectively. After each electrophoresis, the protein in the gel was transferred to a PVDF (polyvinylidene disulfide) membrane (Immobilon-P-transfer membrane, Millipore Corp.), and then dyed in a Coomassie Blue R-250 solution (0.1% of a 50% methanol solution). The band of the AST protein at a place near to about 30 kD was cut out, and the amino acid sequence of the AST protein was determined.

Consequently, with respect to the recombinant AST expressed in the insect cells, an amino acid sequence starting from I-L-G-G was identified under the reducing condition. Three kinds of bands were identified under the non-reducing condition. When the three kinds of the proteins were named as (a), (b) and (c) sequentially from the protein having the largest molecular weight, the contents of (a), (b) and (c) were 8%, 87% and 5%, respectively, which were estimated by the dyeing of silver. The bands were cut out, and then the amino acid sequences of the proteins were determined. The determined amino acid sequences were (a) an amino acid sequence starting from N-S-G-N-L-E-I-N and I-L-G-G-T-E-A-E in equivalent moles, respectively, (b) an amino acid sequence starting from D-Q-A-A-A-N-W-L and I-L-G-G-T-E-A-E in equivalent moles, respectively, and (c) an amino acid sequence starting from I-L-G-G-T-E-A-E. The previously reported AST is the protein having the structure (c).

From the above-described results, it was found that the main band (87%) of the purified rAST had the structure (b) wherein a propeptide moiety starting from the D-Q-A-A-A-N-W-L was bound to a trypsin-like protein moiety starting from the I-L-G-G-T-E-A-E via a disulfide bond. The structure has the activity.

On the other hand, with respect to the AST purified from the sputum, an amino acid sequence starting from the already reported I-L-G-G was identified under the reducing condition. Meanwhile, the peaks of amino acids could be identified, respectively, in addition to the I-L-G-G under the non-reducing condition. The sequence was read as I-N-E-Blank-G-A-G-P (the first I was the same as that of the main peak). The sequence coincided with the amino acid sequence of the human AST, starting from 1 at the 170^{th} position of the propeptide. Thereby, it was found for the first time that the natural AST originated from the sputum mainly had a structure wherein the propeptide was bound via a disulfide bond, similarly to the recombinant AST. It was clarified that the natural AST had a structure in which the cysteine of the disulfide bond on the propeptide (light chain) side was cysteine at the 173^{rd} position and in which the trypsin-like enzyme moiety (heavy chain) was bound to the cysteine moiety corresponding to cysteine at the 292^{nd} position by a structure homology comparison with the other trypsin substance via the disulfide bond.

### Example 4

### Preparation of Various Recombinant Human PAR-Expressing Cells

(1) Acquisition of PAR cDNA and Preparation of PAR cDNA-Containing Transfer Vector

RNA was extracted from normal human airway epithelial cells, and cDNA was synthesized using a cDNA synthesis kit (made by Amersham Pharmacia Biotech. Corp.). PAR-1 was amplified from primers PAR1F (SEQ ID NO: 7) and PAR1R (SEQ ID NO: 8) by a PCR, and PAR-2 was amplified from primers PAR2F (SEQ ID NO: 9) and PAR2R (SEQ ID NO: 10) by a PCR. PAR-3 was also amplified from primers PAR3F (SEQ ID NO: 11) and PAR3R (SEQ ID NO: 12) by a PCR. PAR-4 was further amplified from primers PAR4F (SEQ ID NO: 13) and PAR4R (SEQ ID NO: 14) with a 1/100 amount of a human prostate gland/originated cDNA (purchased from Clontech) by a PCR. The PCR reaction solution was subjected to an agarose gel electrophoresis. Consequently, the band of the cDNA fragment of PAR-1, PAR-2, PAR-3 or PAR-4 was recognized. The DNA fragment was extracted and purified from the gel with a DNA purification kit (using QIAE XII made by QIAGEN Corp.). The purified DNA fragment was cleaved with EcoRI, BamHI (Bgl II with respect to PAR-4), and then ligated to a fragment obtained by cleaving a baculovirus transfer vector pVL 1393 (Invitrogen Corp.) with restriction enzymes EcoRI and BamHI. The obtained reaction solution was used to transform Escherichia coli JM 109, and plasmids were extracted from several transformant colonies. Several clones of the extracted plasmids were purified (using a plasmid extraction kit made by QIAGEN Corp.). The DNA sequences were determined using the purified plasmids with a fluorescence automatic sequencer made by ABI Corp. Consequently, it was found that the DNA fragments encoded the PAR.

(2). Preparation of Recombinant Baculovirus for Expressing PAR

BaculoGold™ transfection kit made by PharMingen Corp. was used for preparing a recombinant baculovirus. Insect cells Sf9 were sowed on a 35 mm dish in a density of 1×10⁶ cells/mL and adhered at room temperature for 30 minutes. On the other hand, 0.5 mg of BaculoGold™ DNA and 2 μg of pVL-PAR plasmid DNA were mixed with each other, left at room temperature for 5 minutes, and then well mixed with 0.5 mL of a transfection buffer B (25 mM HEPES, pH 7.1, 125 mM CaCl₂, 140 mM NaCl). Then, the culture medium of the sowed Sf9 cells was taken out and then mixed with 0.5 mL of a transfection buffer A (Grace culture medium, containing 10% bovine serum). A solution of DNA in the transfection buffer B was gradually dropped on the mixture to carry out the cotransfection, and incubated at 27° C. for 4 hours. Then, the culture medium was replaced by a new Grace culture medium (containing 10% bovine serum). Five days later, the culture supernatant containing the recombinant viruses was recovered. 100 μL of the culture supernatant and 200 μm of EX-CELL 405 culture medium were prepared, and then used to infect the Sf9 cells (1×10⁶ cells/dish) at room temperature for one hour. The supernatant was taken out, dropwisely mixed with 2 mL of a 1% SeaPlaque agarose-containing Grace culture medium (42° C.), and left at room temperature for 10 minutes until to solidify the agarose. Then, the mixture was cultured at 27° C. for 5 days. On the fifth day a neutral red/PBS (Phosphate Buffered Saline) solution (0.1 g/L) was added in a volume of 1 mL/dish, and on the sixth day the virus plaques were observed and identified. Several single virus plaques were taken out together with the agarose with a Pasteur pipette, and then mixed with Grace culture medium to diffuse the viruses in the culture medium. The single plaque-originated virus liquid was used to infect the Sf9 cells (cultured in a 25 cm² flask) cultured in the single layer. The infected cells were cultured at 27° C. for 5 days, and the virus liquid was then recovered. 1 mL of the virus liquid was placed in a 1.5 mL Eppendorf tube and then centrifuged at 15,000 rpm at 4° C. for 30 minutes to precipitate the virus particles. The precipitates were suspended in 200 μL of a TE (10 mM Tris, 0.1 mM EDTA) buffer solution, mixed with 50 μL of Lysis buffer solution (10% SDS, 1 mM EDTA), and incubated at 60° C. for 20 minutes. Further, phenol/chloroform extraction and ethanol precipitation were performed to recover the virus DNA in the reaction solution. One-tenth of the recovered virus DNA was used to carry out a PCR amplification with primers BacF (SEQ ID NO: 15) and BacR (SEQ ID NO: 16). Thus, it was identified that PARcDNA was contained in the DNA. The recombinant baculovirus for expressing the PAR was named VL-PAR. The virus liquid was properly diluted and then subjected to a plaque assay and the check of the titer. Further, the virus liquid was used to infect the Sf9 cells to obtain the virus liquid having a high concentration of about 1-5×10⁷ pfu (plaque forming unit)/mL. The virus liquid was stored at 4° C. and −8° C.

(3). Expression of PAR in Insect Cells with Recombinant Baculovirus

The above-obtained recombinant baculovirus VL-PAR for expressing the PAR was used to infect 1.0×10⁷ Sf9 cells sowed on a 75 cm² flask at an MOI=2, and was then left and stationally cultured at 27° C. for 40 hours. After the culture, the infected cells were recovered and then subjected to a calcium influx experiment to-confirm the function of the PAR.

### Example 5

### Evaluation of Calcium Influx Due to Human AST with PAR-Expressing Insect Cells

The recovered infected cells (about 5×10⁶ cells) were once washed with HEPES-Tyrode buffer solution-Ca²⁺ (−) pH 7.4, and then suspended in 5 mL of HEPES-Tyrode buffer solution (containing Fura2-AM, 1 μg/mL) to load the Fura2-AM on the cells at 27° C. for 30 minutes. The suspension was centrifuged at 800 rpm at room temperature for 5 minutes to recover the cells. The cells were twice washed with HEPES-Tyrode buffer solution-Ca²⁺ (−) pH 7.4 and then resuspended in 5 mL of HEPES-Tyrode buffer solution-Ca²⁺ (−) pH 7.4. The cell suspension was mixed with 5 μL of 1M CaCl₂ to give a final concentration of 1 mM CaCl₂, and then incubated in a shaded state at room temperature for 5 minutes. The prepared Fura2-AM-loaded infected cells were dispensed into a 96 well plate in a volume of 100 μL/well, further subjected to the dispensation of HEPES-Tyrode buffer solution-C²⁺ (+) pH 7.4 in a volume of 80 μL/well, and then assayed with a Fluorescence Drug Screening System (made by Hamamatsu Photonics Co.). Namely, [intensity of 500 nm fluorescent light due to 340 nm exciting light]/[intensity of 500 nm fluorescent light due to 380 nm exciting light] (reflecting the concentration of calcium in the cells) was assayed.

AST (0.2 to 2 units/mL), trypsin (originated from bovine pancreas, 0.1 to 10 units/mL), human thrombin (0.1 to 10 units/mL), or SFLLRNamide (agonist peptide for PAR-1, 2), or SLIGRL (agonist peptide for PAR-2, 10 to 100 μM) was added to the cells to stimulate the cells, and the calcium concentrations in the cells were assayed with the passage of time. Consequently, the specific calcium influx was observed in the insect cells infected with the recombinant baculovirus for expressing the PAR. From the result, it was proved that the function-having PAR was expressed in the insect cells.

The Ca²⁺ influxes of PAR-1 to PAR-4 are shown below, respectively.

For PAR-1, the Ca²⁺ influx was observed at AST (2 units/mL), trypsin (0.1 to 10 units/mL), thrombin (0.1 to 10 units/mL), and SFLLRNamide (10 to 100 μM).

For PAR-2, the Ca²⁺ influx was observed at AST (2 units/mL), trypsin (0.1 to 10 units/mL), thrombin (0.1 to 10 units/mL), SFLLRNamide (10 to 100 μM), and SLIGRL (10 to 100 μM).

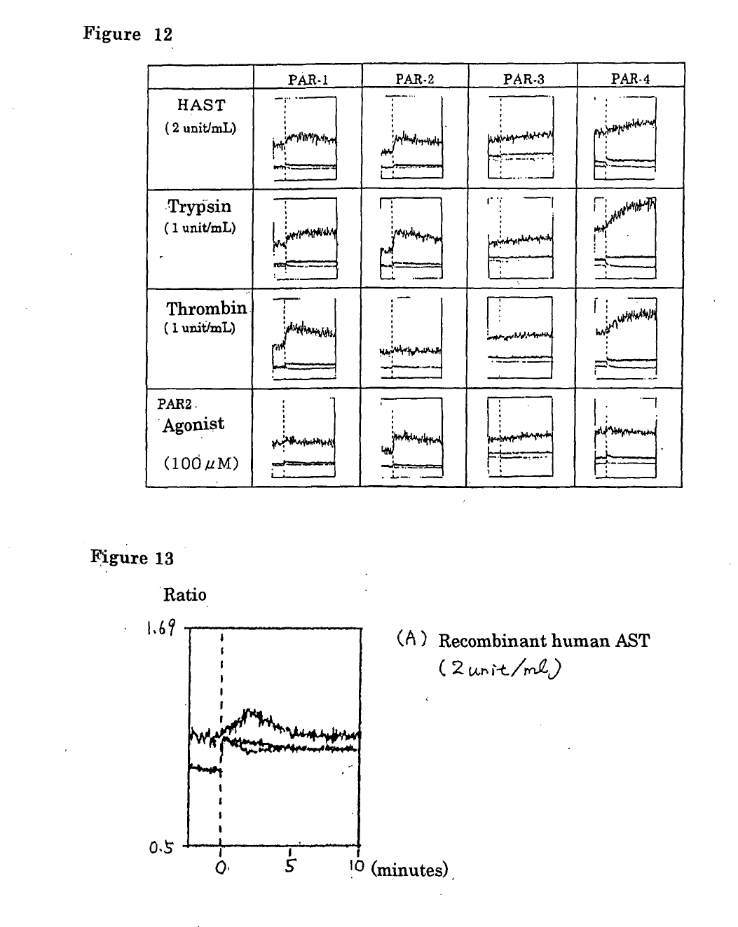

For PAR-4, the Ca²⁺ influx was observed at AST (2 units/ml), trypsin (0.1 to 10 units/mL), and thrombin (0.1 to 10 units/mL) (see FIG. 12).

### Example 6

### Experiment for Calcium Influx Due to AST with Airway Epithelial Cell

(1) Experiment for Calcium Influx Due to AST with Normal Human Airway Epithelial Cell

The Ca²⁺ influx due to the AST was examined with confluent normal epithelial cells adhered to three 10 cm dishes (purchased from BioWhittaker Corp.). The cells were peeled with a 10 mM EDTA/HEPES-Tyrode buffer solution for 10 minutes, recovered by a centrifugal treatment at 800 rpm at room temperature for 5 minutes, and then resuspended in 5 mL of HEPES-Tyrode buffer solution-Ca²⁺ (−) pH 7.4. The recovered cells (about 1.2×10⁶ cells) were suspended in 5 mL of HEPES-Tyrode buffer solution (1 μg/mL, containing Fura2-AM) to load the Fura2-AM to the cells at 37° C. for one hour. The cells were recovered by a centrifugal treatment at 800 rpm at room temperature for 5 minutes, twice washed with HEPES-Tyrode buffer solution-Ca²⁺ (−) pH 7.4, and then resuspended in 1.3 mL of HEPES-Tyrode buffer solution-Ca²⁺ (−) pH 7.4. The cell suspension was mixed with 5 μL of 1M CaCl₂ to give a final concentration of 1 mM CaCl₂, and then incubated in a shaded state at room temperature for 5 minutes. The prepared Fura2-AM-loaded infected cells were dispensed into a 96 well plate in a volume of 100 μL/well, further subjected to the dispensation of HEPES-Tyrode buffer solution-Ca²⁺ (+) pH 7.4 in a volume of 80 μL/well, and then assayed with Fluorescence Drug Screening System (Hamamatsu Photonics Co.). Namely, [intensity of 500 nm fluorescent light due to 340 nm exciting light]/[intensity of 500 nm fluorescent light due to 380 nm exciting light] (reflecting the concentration of calcium in the cells) was assayed.

AST (2 units/mL), bovine pancreas-originated trypsin (1 to 100 units/mL), human thrombin (1 to 100 units/mL), SFLLRNamide, or SLIGRL (10 to 100 μg) were added to stimulate the cells, and calcium concentrations in the cells were then assayed with the passage of time.

Consequently, a Ca²⁺ influx was observed at AST (2 units/mL), trypsin (10 to 100 units/mL), and SFLLRNamide (100, M) (see FIG. 13).

(2) Experiment for Calcium Influx Due to AST with Airway Epithelial Cell Strain

The Ca²⁺ influx due to the AST was examined with respect to an airway epithelial cell strain BEAS-2B. The confluent cells adhered to six 10 cm dishes were used. The cells were peeled with a 10 mM EDTA/HEPES-Tyrode buffer solution for 10 minutes, recovered by a centrifugal treatment at 1,500 rpm at room temperature for 5 minutes, and then resuspended in 10 mL of HEPES-Tyrode buffer solution-Ca²⁺ (−) pH 7.4. The recovered cells (about 1.1×10⁷ cells) were suspended in 11 mL of HEPES-Tyrode buffer solution (1 μg/mL, containing Fura2-AM) to load the Fura2-AM at 37° C. for 30 minutes. The cells were recovered by a centrifugal treatment at 1,500 rpm at room temperature for 5 minutes, twice washed with HEPES-Tyrode buffer solution-Ca²⁺ (−) pH 7.4, and then resuspended in 11 mL of HEPES-Tyrode buffer solution-Ca²⁺ (−) pH 7.4. The cell suspension was mixed with 11 mL of 1M CaCl₂ to give a final concentration of 1 mM CaCl₂, and then incubated in a shaded state at room temperature for 5 minutes. The prepared Fura2-AM-loaded infected cells were dispensed into a 96 well plate in a volume of 100 μL/well, further subjected to the dispensation of HEPES-Tyrode buffer solution-Ca²⁺ (+) pH 7.4 in a volume of 80 μL/well, and then assayed with Fluorescence Drug Screening System (Hamamatsu Photonics Co.). Namely, [intensity of 500 nm fluorescent light due to 340 nm exciting light]/[intensity of 500 nm fluorescent light due to 380 nm exciting light] (reflecting the concentration of calcium in the cells) was assayed.

AST (0.2 to 2 units/mL), bovine pancreas-originated trypsin (0.1 to 100 units/mL), human thrombin (0.1 to 100 units/mL), or SFLLRNamide, or SLIGRL (PAR against peptide, 10 to 100 μM) were added to stimulate the cells, and calcium concentrations in the cells were then assayed with the passage of time.

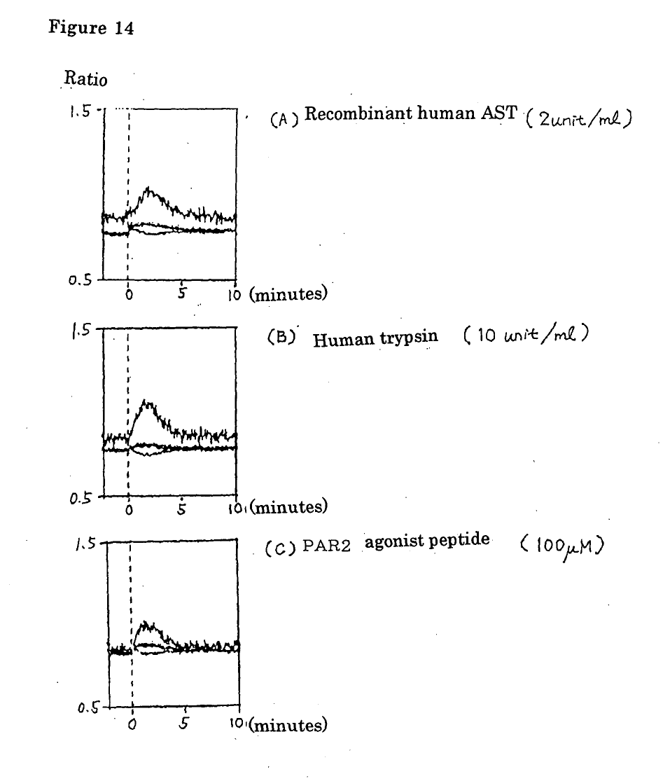

Consequently, the Ca²⁺ influx was observed at AST (2 units/mL), trypsin (10 to 100 units/mL), and SFLLRNamide (100 μM) (see FIG. 14).

### Example 7

### IL-8 Release from Airway Epithelial Cell Strain (BEAS-2B) Due to HAST

1). Cells

Tests were carried out with BEAS-2B (airway epithelial cell strain, purchased from ATCC). LHC-9 (produced by Biofluid Corp.) was used for usual cultures. When the cells were used for the test, the cells were suspended in LHC-8 (not containing hydrocortisone, produced by Biofluid Corp.), sowed on a 96 well plate (made by Falcon Corp.) coated with collagen (produced by Collagen Corp.)/BSA (produced by Boehringer Mannheim GmbH)/fibronectin (Sigma Corp.) in a density of 5.0×10⁴ cells/cm², and then cultured for 24 hours. After the normal take of the cells was identified, the cells were treated with a drug.

2) Drug Treatment

HAST (100 mU/mL) and TNF— a (produced by R & D) (1 mg/mL) were reacted with a prescribed concentration of benzamidine or leupeptin in LHC-8 (not containing hydrocortisone) on ice for 30 minutes, and then heated at 37° C. The heated reaction solution was wholly exchanged by the cell culture solution in the wells to start the drug treatment. After the culture for 24 hours, the culture supernatant was recovered and stored at −80° C., until the assay of IL-8 production was carried out.

3) IL-8 Concentration Assay

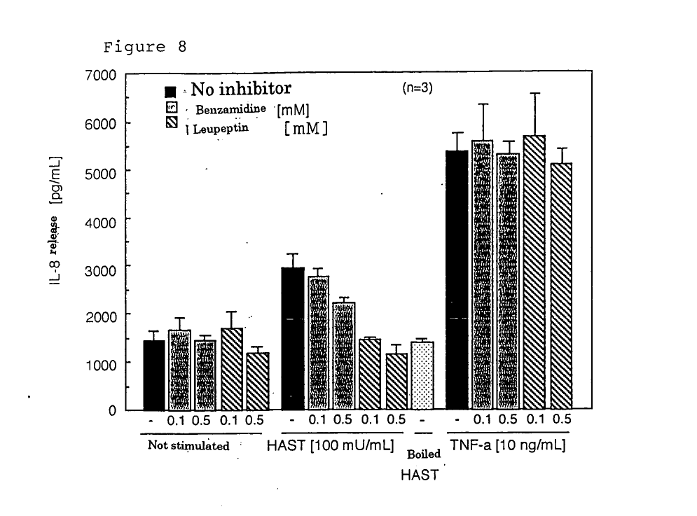

The concentration of IL-8 in the culture supernatant was assayed by a sandwich ELISA method using a commercially available antibody. Mouse MAb Anti-Human IL-8 Capture (produced by Biosource Corp.) adjusted with PBS in a concentration of 1 μg/mL was added to a 96 well EIA plate (made by Maxi Sorp. Nunc Corp.) in a volume of 50 μL/well, and then allowed to stand at 4° C. overnight to immobilize the antibody. The immobilized antibody was washed with 250 μL/well of a washing buffer solution (PBS containing 0.05% Tween 20 (produced by BioRad Corp.)) five times, mixed with 250 μL/well of a blocking buffer solution (PBS containing 1% BSA (produced by Boehringer Mannheim GmbH) and 0.05% sodium azide), and then allowed to stand at 4° C. overnight to carry out the blocking treatment. The blocked product was washed with 250 μL/well of a washing buffer solution five times, mixed with 50 μL/well of the culture supernatant diluted with the blocking buffer solution 50 times or with IL-8 standard prepared with the blocking buffer solution, and reacted at room temperature for one hour. The reaction product was washed with 250 μL/well of the washing buffer solution five times, mixed with 50 μL/well of Mouse MAb Anti-Human IL-8 biotin (produced by Biosource Corp.) prepared with a diluting buffer solution (PBA containing 1% BSA and 0.05% of Tween 20) in a concentration of 0.05 μg/mL, and reacted at room temperature for one hour. The reaction product was washed with 250 μL/well of the washing buffer solution five times, mixed with 50 μL/well of HRP-streptavidin (produced by Genzyme Corp.) diluted with the diluting buffer solution 6,000 times, and reacted for 15 minutes. The reaction product was washed with 250 μL/well of the washing buffer solution five times, and then treated with 100 μL/well of TMB Microwell peroxidase substrate (produced by KPL Corp.) for about 15 minutes to develop the color. 100 μL/well of 1M sulfuric acid was added to stop the color-developing reaction, and then the absorbance of the product at 450 nm was immediately assayed with a ThermoMax microplate reader (made by Molecular Devices Corp.). The absorbance of each sample was regressed to a standard curve made from the absorbances of standards to calculate the concentration of IL-8 in the sample. The results are shown in FIG. 8. Consequently, it was shown that the release of the IL-8 from the airway epithelial cells caused by the HAST was inhibited by inhibiting the activity of the HAST with the protease inhibitors.

### Example 8

### Transcriptional Activation of IL-8 Promoter by HAST

1) Cloning of IL-8 Promoter Domain

About 1.5 Kb and 162 bp of the promoter domains (Mukaida N. et al., J. Immunol., 143: 1366-1371, 1989) of IL-8 were cloned with LA-PCR (produced by Takara Shuzo Co.) using human genome DNA as a template, a forward primer: IL8-1481; CCCAGATCTGAATTCAGTAACCCAGGCATTATTTTATC, a forward primer: IL8-162; AACTTTGGATCCACTCCGTATTTGATAAGG, a reverse primer: IL8LUC-R; CATGTTTACACACAGTGAGAATGGTTCCTTCC. The PCR was carried out at 98° C. for 20 sec, at 58° C. for one minute and at 68° C. for 5 minutes in 25 cycles. The obtained DNA fragments of the promoter domains of about 1.5 Kb and about 0.25 Kb were named IL8-1481 and IL8-162, respectively.

2) Construction of IL-8 Reporter Plasmid

A DNA fragment containing from ATG (translation-starting codon) to NarI site in the luciferase gene of pGL3-Basic (produced by Promega Corp.) was amplified by a PCR method using a forward primer: IL8LUC-F; CTGTGTGTAAACATGGAAGACGCCAAAAACATAAA and a reverse primer: LUC2087R; CGGGAGGTAGATGAGATGTG. This luciferase gene N-terminal DNA fragment was ligated to the IL8-1481 and the IL8-162 by a PCR method to obtain the PCR fragments IL8-1481-Luc (about 2 Kb) and IL8-162-Luc (about 800 bp). The PCR fragments were cleaved with BamHI and NarI and then inserted into the upper stream of the luciferase gene with the BglII and NarI of the pGL3-Basic to construct the reporter plasmids pGL-IL8-1418, pGL3-IL8-162.

3) Cells

Tests were performed with BEAS-2B (airway epithelial cell strain, purchased from ATCC). An ordinary culture was carried out with LHC-9 (produced by Biofluid Corp.). When the cell strain was used for the test, the cell strain was suspended in LHC-8 (not containing hydrocortisone, produced by Biofluid Corp.), sowed on a 96 well plate (produced by Falcon Corp.) coated collagen (produced by Collagen Corp.)/BSA (produced by Boehringer Mannheim Corp.)/fibronectin (produced by Sigma Corp.) in a density of 2.0×10⁴ cells/cm², and then cultured 24 hours. After it was identified that the cells normally took, the transient gene introduction was carried out.

4) Transient Gene Introduction

Transient gene introduction was carried out with LipofectAmine PLUS reagent (produced by Gibco Corp.). The culture solution in the 96 well plate was replaced by 50 μL of fresh LHC-8 (not containing hydrocortisone, produced by Biofluid Corp.). A transfection culture medium containing 0.01 μg of a control plasmid, 0.09 μg of a reporter plasmid (pGL3-Basic or pGL3-IL8-1481 or pGL3-IL8-162), 0.5 μL of LipofectAmine reagent (produced by Gibco Corp.) and 0.5 μL of PLUS reagent (produced by Gibco Corp.) in 20 μL of LHC-8 (not containing hydrocortisone, produced by Biofluid Corp.) was added in a volume of 20 μL/well to start the gene introduction. Then, the culture was carried out in a CO₂ incubator for 48 hours.

5) Drug Treatment

48 Hours later from the gene introduction, a drug treatment was carried out. The culture solution in the wells was replaced by LHC-8 (not containing hydrocortisone, produced by Biofluid Corp.) containing HAST (300 mU/mL) or TNF-α (produced by R & D Corp.)(1 ng/mL), and the culture was performed for 3 hours. Subsequently, the culture solution was removed, and the left cells were once washed with PBS and then supplied for the assay of luciferase activity.

6) Assay of Luciferase Activity

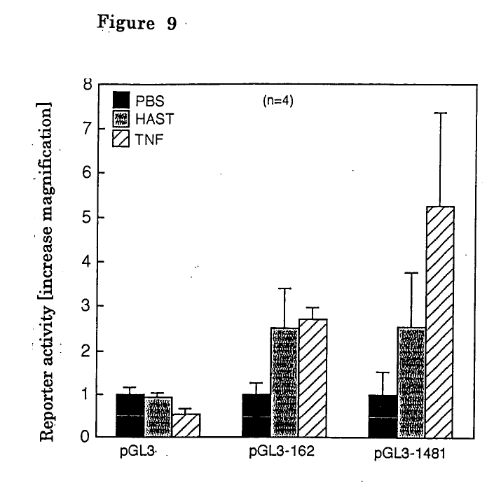

The luciferase activity was assayed with Pica Gene Dual•Renilla reniformis (produced by Toyo Ink Co.,). The cells were treated with HAST or TNF-α for three hours, subjected to the removal of the culture solution, once washed with PBS, and then lysed with 20 μL of a cell-lysing agent at room temperature for 15 minutes. The cell-lysed solution was recovered with a 1.5 mL micro tube, and then centrifuged at 15,000 rpm for 10 minutes. The supernatant was collected as a sample, and 20 μL of the sample was added to a black 96 well plate (made by Sumitomo Bakelite Co.). First, 100 μL of Pica Gene luminescent reagent II was added, and the luminescence due to the firefly luciferase was assayed with a luminometer (LUMINOUS CT-9000D (produced by DIA-IATRON Corp.)). Subsequently, 100 μL of Renilla reniformis luminescent reagent was added, and the luminescence due to the Renilla reniformis luciferase was assayed with the luminometer (LUMINOUS CT-9000D (produced by DIA-IATRON Corp.)). A correction comprising dividing the obtained firefly luciferase activity by the Renilla reniformis luciferase activity was carried out to obtain the reporter activity (Relative Light Unit (R. L. U.)). Further, the reporter activity in each sample was subjected to a data analysis as an increased magnification based on an average value in a non-stimulated group. The results are shown in FIG. 9. From the results, it was clarified that the HAST promoted the production of the IL-8 at a transcription level.

### Example 9

### Acquisition of Mouse AST Counter Part cDNA

(1). Acquisition of Mouse Airway cDNA

The airways of mice (C57 Black) were collected, and then homogenized in 1 mL/airway of ISOGEN. The obtained homogenate was mixed with 0.2 mL of chloroform in a vortex state, allowed to stand at room temperature for 2 to 3 minutes, and then centrifuged at a 12,000 rpm at 4° C. for 10 minutes. The upper layer was transferred into a new tube, mixed with 0.5 mL of isopropyl alcohol, and then allowed to stand at room temperature for 10 minutes. The mixture was centrifuged at 15,000 rpm at 4° C. for 15 minutes to precipitate the total RNA. The obtained pellet was well mixed with 1 mL of 75% ethanol and then centrifuged at 10,000 rpm at 4° C. for 5 minutes. The obtained pellet was dried with air, dissolved in water free from DNase/RNase, and then preserved at −80° C.

The cDNA was synthesized with oligo (dT)₁₂₋₁₈ and 2.5 μg of the obtained whole RNA originated from the mouse airways by the use of SuperScript™ Preamplification System for First Strand cDNA Synthesis Kit (made by GIBCO-BRL Corp.) according to an appended protocol.

(2) Acquisition of AST PCR Fragment from Mouse Airway cDNA

A homology comparison was carried out between the base sequences of human AST and mouse hepsin, and a forward primer and a reverse primer represented by SEQ ID NO: 17 and SEQ ID NO: 18, respectively, were produced. A PCR reaction was performed using the primers and the cDNA originated from the mouse airways as a template by the use of TaKaRa Taq™ polymerase (produced by Takara Shuzo Co.) according to an appended protocol. The PCR product was subjected to an agarose gel electrolysis. Consequently, an amplified band was recognized near to a target 450 bp. The amplified DNA fragments were cut out from the agarose gel, and then inserted into TA cloning vector. Escherichia coli was transformed with the vector to obtain the clone. A plasmid was prepared by an established method, and the base sequence of the inserted DNA fragment was determined. Consequently, it was clarified that not only the mouse Hepsin but also a gene exhibiting the homology with the human AST were cloned.

(3) Acquisition of Full-Length Mouse AST cDNA by 5′-RACE, 3′-RACE

Primers for 5′-RACE (Rapid Amplification of cDNA Ends) and 3′-RACE were synthesized on the partial base sequence obtained in (2). The primer sequences for the 1^{st} RACE are represented by SEQ ID NO: 19 and SEQ ID NO: 20, and the primer sequences for the 2^{nd} RACE are represented by SEQ ID NO: 21 and SEQ ID NO: 22. The 5′-RACE and the 3′-RACE were performed on the sequences with Mouse 15-day Embryo Marathon-Ready™ cDNA (produced by Clontech Corp., Swiss-Webster/NIH embryo). A PCR reaction was carried out with Advantage 2 Polymerase Mix (produced by Clontech Corp.) according to an appended protocol. The PCR product was subjected to an agarose gel electrophoresis, and the about 1.1 bp amplified fragment due to the 5′-RACE and the about 0.6 kbp amplified fragment due to the 3′-RACE were thus obtained. The amplified DNA fragments were cut out from the agarose gel, inserted into TA cloning vectors, which were used to transform Escherichia coli, thus obtaining several clones of the transformants, respectively. Plasmids were prepared from the transformants by an established method, and then the base sequences of the inserted DNA fragments were determined. As the result, it was identified that the base sequence starting from a sequence coincident with the partial base sequence obtained in (2) and having homology with the human AST in the 5′-direction and in the 3′-direction was extended. The determined perfect length mouse AST cDNA sequence and amino acid sequence are represented by SEQ ID NO: 4.

Further, in order to confirm whether mouse AST practically existing in a mouse airway has the same amino acid sequence or not, PCR primers (SEQ ID NO: 23 and SEQ ID NO: 24) recognizing the outside of a gene encoding the amino acid were synthesized. Advantage 2 Polymerase (produced by Clontech Corp.) was used to perform the PCR reaction of a mouse airway cDNA, and the base sequence of the amplified DNA fragment was determined. Consequently, it was found that the amino acid sequence coincided, and the cloning of the airway-originated mouse AST cDNA (SEQ ID NO: 6) was succeeded.

(4) Comparison of Homology with Human AST

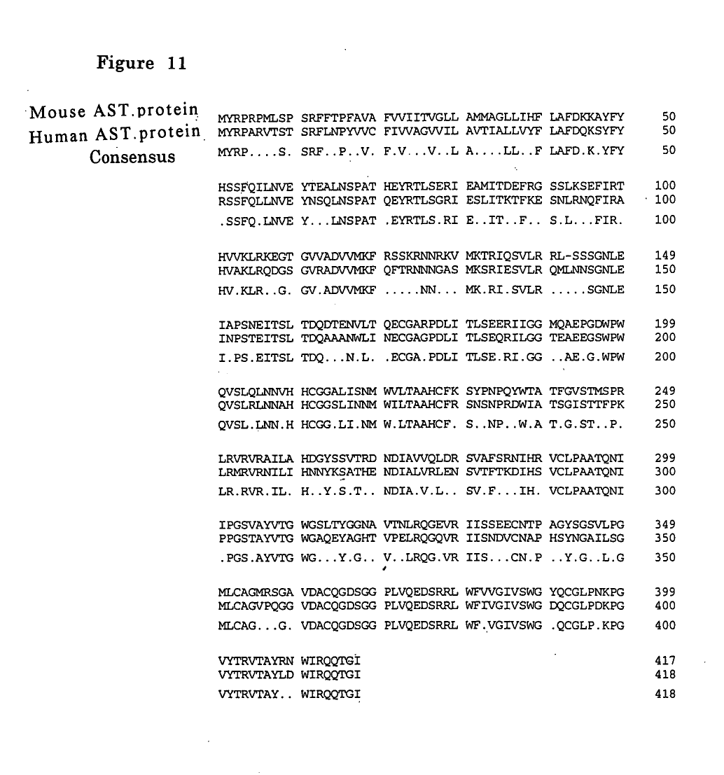

The amino acid homology comparison result between the human AST and the mouse AST is shown in FIG. 11. The homology comparison was carried out with a gene-analyzing software (Gene Works; Teijin System Technology Co.). 66% of the amino acid sequences were totally coincident with each other.

### Example 10

### Acquisition of Rodent AST Counter Part cDNA

(1). Synthesis of Rodent Airway cDNA

The airway of a hamster or guinea pig was picked, and then homogenized in about 5 mL of ISOGEN per 0.5 gram of the airway. The obtained homogenate was centrifuged at 3,000 rpm for 10 minutes, and 1 mL of the supernatant was collected. The supernatant was mixed with 0.2 mL of chloroform in a vortex state, allowed to stand at room temperature for 2 to 3 minutes, and then centrifuged at a 12,000 rpm at 4° C. for 10 minutes. The upper layer was transferred to a new tube, mixed with 0.5 mL of isopropyl alcohol, and then allowed to stand at room temperature for 10 minutes. The mixture was centrifuged at 15,000 rpm at 4° C. for 15 minutes to precipitate the total RNA. The obtained pellet was well mixed with 1 mL of 75% ethanol and then centrifuged at 10,000 rpm at 4° C. for 5 minutes. The obtained pellet was dried with air, dissolved in water free from DNase and RNase, and then preserved at −80° C.

The cDNA was synthesized using oligo (dT)₁₂₋₁₈ and 2.5 μg of the obtained total RNA originated from the airways by the use of SuperScript™ Preamplification System for First Strand cDNA Synthesis Kit (made by GIBCO-BRL Corp.) according to an appended protocol.

(2) Acquisition of AST PCR Fragment from cDNA

A homology comparison was carried out between the base sequences of human AST and mouse hepsin, and a forward primer and a reverse primer represented by SEQ ID NO: 17 and SEQ ID NO: 18, respectively, were produced. A PCR reaction was performed using the primers and the cDNA originated from the airways as a template by the use of TaKaRa Taq™ polymerase (produced by Takara Shuzo Co.) according to an appended protocol. The PCR product was subjected to an agarose gel electrolysis. Consequently, an amplified band was recognized near to a target 450 bp. The amplified DNA fragments were cut out from the agarose gel, and then inserted into TA cloning vector. Escherichia coli was transformed with the vector to obtain the clone. A plasmid was prepared by an established method, and the base sequence of the inserted DNA fragment was determined. Consequently, it was clarified that a gene exhibiting the homology with the human AST were cloned.

(3) Acquisition of Full-Length Rodent AST cDNA by 5′-RACE 3′-RACE

Primers for 5′-RACE and 3′-RACE were synthesized on the partial base sequence obtained in (2). On the other hand, the cDNA for the 5′-RACE and the 3′-RACE was synthesized with 1 μg of the total RNA collected from the airways by the use of SMART-RACE Kit (made by Clontech Corp.). The 5′-RACE and the 3′-RACE were performed. A PCR reaction was carried out with Advantage 2 Polymerase Mix (produced by Clontech Corp.) according to an appended protocol. The PCR products obtained by the 5′-RACE and the 3′-RACE were subjected to an agarose gel electrophoresis. The main amplified DNA fragments were cut out from the agarose gel, inserted into TA cloning vectors (produced by Invitrogen Corp.), which were used to transform Escherichia coli, thus obtaining several clones of the transformants, respectively. Plasmids were prepared from the transformants by an established method, and then the base sequences of the inserted DNA fragments were determined. As the result, it was identified that the base sequence starting from a sequence coincident with the partial base sequence obtained in (2) and having homology with the human AST in the 5′-direction and in the 3′-direction was extended. The determined perfect length rodent AST cDNA sequence and amino acid sequence are shown. The guinea pig: SEQ ID NO: 36 and 37, the hamster: SEQ ID NO: 38 and 39.

### Example 11

### Acquisition of Mammalian AST Counter Part cDNA

(1). Acquisition of Mammalian Airway cDNA

The airway of rabbit, dog, pig, cattle, or Macaca irus was picked, and then homogenized in about 5 mL of ISOGEN per 0.5 g of the airways. The obtained homogenate was centrifuged at 3,000 rpm for 10 minutes, and 1 mL of the supernatant was collected. The supernatant was mixed with 0.2 mL of chloroform in a vortex state, allowed to stand at room temperature for 2 to 3 minutes, and then centrifuged at a 12,000 rpm at 4° C. for 10 minutes. The upper layer was transferred into a new tube, mixed with 0.5 mL of isopropyl alcohol, and then allowed to stand at room temperature for 10 minutes. The mixture was centrifuged at 15,000 rpm at 4° C. for 15 minutes to precipitate the total RNA. The obtained pellet was well mixed with 1 mL of 75% ethanol and then centrifuged at 10,000 rpm at 4° C. for 5 minutes. The obtained pellet was dried with air, dissolved in water free from DNase and RNase, and then preserved at −80° C.

The cDNA was synthesized with oligo (dT)₁₂₋₁₈ and 2.5 μg of the obtained total RNA originated from the airways by the use of SuperScript™ Preamplification System for First Strand cDNA Synthesis Kit (made by GIBCO-BRL Corp.) according to an appended protocol.

(2) Acquisition of AST PCR Fragment from Airway cDNA

A homology comparison was carried out between the base sequences of human AST and rodent AST, and a forward primer represented by ASTS652F (SEQ ID NO: 40) and ASTS721F (SEQ ID NO: 41) and a reverse primer represented by ASTS1166 (SEQ ID NO: 42) and ASTS1211 (SEQ ID NO: 43) were produced. A PCR reaction was performed with the primers and the cDNA originated from the mammalian airways as a template by the use of Pyrobest DNA polymerase (produced by Takara Shuzo Co.) according to an appended protocol. The PCR product was subjected to an agarose gel electrolysis. Consequently, an amplified band was recognized from about 475 bp to about 590 bp. The amplified DNA fragment was cut out from the agarose gel, and then inserted into TA cloning vector. Escherichia coli was transformed with the vector to obtain the clone. A plasmid was prepared by an established method, and the base sequence of the inserted DNA fragment was determined. Consequently, it was clarified that a gene fragment exhibiting the homology with the human AST were cloned.

(3) Acquisition of Full-Length Mammalian AST cDNA by 5′-RACE, 3′-RACE

With respect the rabbit AST, primers for 5′-RACE and 3′-RACE were synthesized on the partial base sequence obtained in (2). With respect to the simian, dog, swine or bovine AST, SEQ ID NO: 40 for 3′-RACE and SEQ ID NO: 43 for 5′-RACE were used as primer sequences for the 1^{st} RACE, and SEQ ID NO: 41 for 3′-RACE and SEQ ID NO: 42 for 5′-RACE were used as primer sequences for the 2^{nd} RACE. On the other hand, the cDNA for the 5′-RACE and the 3′-RACE was synthesized with 1 μg of the total RNA collected from each mammalian airway by the use of SMART-RACE Kit (made by Clontech Corp.). The 5′-RACE and the 3′-RACE were performed. A PCR reaction was carried out with Advantage 2 Polymerase Mix (produced by Clontech Corp.) according to an appended protocol. The PCR products obtained by the 5′-RACE and the 3′-RACE were subjected to an agarose gel electrophoresis. The main amplified DNA fragments were cut out from the agarose gel, inserted into TA cloning vectors (produced by Invitrogen Corp.), which were used to transform Escherichia coli, thus obtaining several clones of the transformants, respectively. Plasmids were prepared from the transformants by an established method, and then the base sequences of the inserted DNA fragments were determined. As the result, it was identified that the base sequence starting from a sequence coincident with the partial base sequence obtained in (2) and having homology with the human AST in the 5′-direction and in the 3′-direction was extended. Each determined perfect length mammalian AST cDNA sequence and amino acid sequence are shown. The pig: SEQ ID NO: 26 and 27, monkey: SEQ ID NO: 28 and 29, dog: SEQ ID NO: 30 and 31, cattle: SEQ ID NO: 32 and 33, rabbit: SEQ ID NO: 34 and 35.

### Example 12

### Assay of Enzymatic Activities of Various Mammalian ASTs

(1) Preparation of Plasmids for Expressing Various Mammalian AST Animal Cells

A forward primer and a reverse primer were prepared on gene sequences encoding the N-terminal and C-terminal of each mammalian AST, and a cDNA encoding the full-length AST was prepared by a PCR amplification using the forward primer, the reverse primer, Pyrobest DNA polymerase (produced by Takara Shuzo Co.), and an airway cDNA as a template. On the other hand, a product obtained by cleaving pcDNA3 (produced by Invitrogen Corp.) with restriction enzymes NruI and KpnI, and the promoter domain of Elongation Factor-1a (EF-1 a) from human genome were amplified by a PCR using a forward primer: GACTTCGCGACGTGAGGCTCCGGTGCCCGTC and a reverse primer: GACTGGTACCAAGCTTTTCACGACACCTGAAATGGAAG. The amplified fragment was cleaved with restriction enzymes NruI and KpnI, and then inserted into the above-described plasmid vector to construct the pEF9 expression vector having the promoter of the human EF-1α. The PCR-amplified ATS fragment was inserted into a multi-cloning site in the downstream of the human EF-1α promoter of the above-described pEF9 expression vector to prepare each animal AST expression plasmid. The base sequences were identified with an automatic sequencer.

(2) Enzymatic Activity Evaluation by Transient Expression in Animal Cultured Cell

Human fetal kidney-originated 293 EBNA cells (Invitrogen Corp.) were cultured in a 10 cm schale, and the about 5.8×10⁶ cells were transfected with 28 μg of each expression plasmid prepared in (1) in 56 μL of LipofectAMINE 2000 (produced by GibcoBRL Corp.) according to an appended manual. Three days later, the cells were lysed and recovered in 5 mL of M-PER (produced by PIERCE Corp.) and then mixed with 5 mL of a buffer solution (50 mM Tris-HCl, 0.01% BSA, pH=8.6) for AST activity assay to give the cell lysate. The cell lysate was further diluted with the buffer solution for the AST activity assay ten times, and then supplied for the activity assay. 40 μL of the above-described assay sample was mixed with 20 μL of a synthetic substrate solution (a solution obtained by diluting a 10 mM Boc-Phe-Ser-Arg-MCA DMSO (dimethyl sulfoxide) solution with the buffer solution for the AST activity assay 50 times), and the increase of the fluorescence intensity per minute was assayed (fluorescence wavelength: 460 nm, excitation wavelength: 380 nm) to determine the enzymatic activity. The results are shown in the next table.

| | Cell lysate | Sup Fluorescence |
|---|---|---|
| | Fluorescence intensity ratio | intensity ratio |
| | (Human:100) | (Human:100) |
| Human | 100 | 100 |
| Monkey | 250 | 150 |
| Pig | 92 | |
| Dog | 40 | |
| Cattle | 16 | |
| Rabbit | 30 | |

(3) Preparation of Recombinant Baculovirus for Insect Cell Expression of Mammalian AST

With respect to mouse AST, dog AST, or simian AST, Bac-to-Bac System (produced by GIBCO-BRL Corp.) was used according to an appended protocol to prepare a recombinant Bacmid for insect cell expression, and the Sf9 cell strain was transfected with the recombinant Bacmid to obtain the recombinant baculovirus.

(4) Massive Expression of Mammalian AST by Insect Cell Expression and Enzyme Activity Assay

An insect cell strain Tn5 was massively cultured, and then infected with the above-described baculovirus. The infected insect cell strain was cultured for three days to express the recombinant mammalian AST. The expressed cell pellet was subjected to the same operations as in Example 1 to obtain the purified recombinant mammalian AST. The cleavage activity of the obtained recombinant mammalian AST was evaluated with three kinds of trypsin-like synthetic substrates. Consequently, it was found that the recombinant mammalian AST had an activity to cleave the synthetic substrates: Boc-Phe-Ser-Arg-pNA, Boc-Ser-Glu-Gln-Arg-pNA, and Boc-Ser-Lys-Gly-Arg-pNA similarly to that of HAST and thereby had the same trypsin-like activity as that of the HAST.

### Example 13

### Analysis of Mucus Glycoprotein Synthesis by Addition of AST

(1) Synthesis of Mucus Glycoprotein with Various EGFR Ligands

1) Cell Preparation

NCI-H292 cells (human pulmonary mucoepidermoid tumor cell strain, purchased from ATCC) were suspended in 10% FCS (Fetal Calf Serum)-containing RPMI-1640 (produced by Gibco Corp.), sowed on a Lab-Tek 8 well chamber slide (produced by Nunc Corp.) in a density of 6.0×10⁴ cells/cm², cultured for 72 hours, subjected to the replacement of the culture solution by RPMI-1640 containing 0.1% of BSA, and further cultured for 24 hours.

Drug Treatment

2) Drug Treatment

On the day of the treatment, the culture solution of each well was replaced by RPMI-1640 (containing 0.1% BSA) containing EGF (5 ng/mL, produced by Becton Dickinson Corp.), HB-EGF (5 ng/mL, produced by R & D Corp.), TGF-α (5 ng/mL, produced by Peprotech Corp.), or PBS to start the treatment. Then, the cells were incubated at 37° C. for 24 hours.

3) AB-PAS (Alcian Blue-Periodic Acid/Schiff) Staining

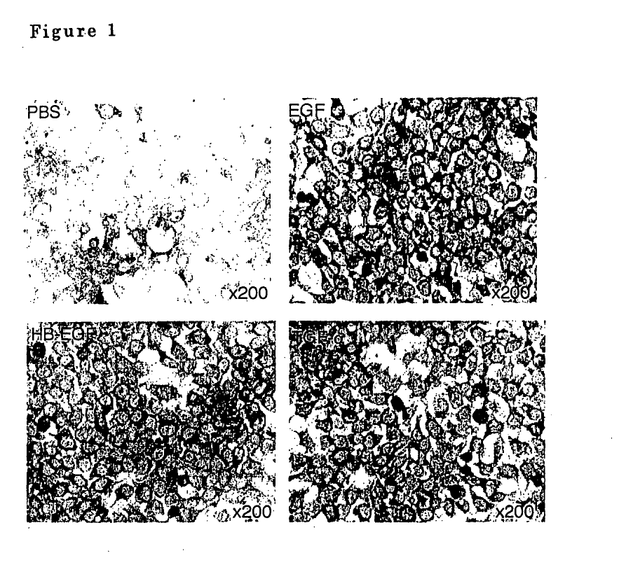

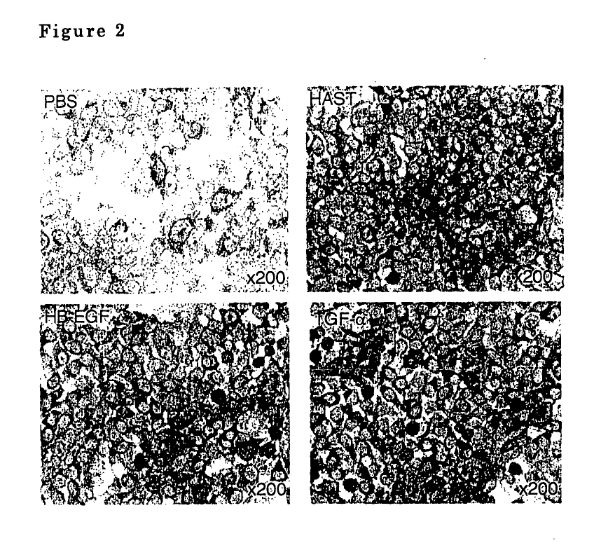

After the prescribed 24 hours treatment, the culture supernatant was removed. The residue was once washed with PBS, mixed with 4% paraformaldehyde/PBS, and then allowed to stand at room temperature for one hour to immobilize the cells. The immobilized cells were twice washed with Milli Q water and then stained with AB-PAS. The staining was carried out according to a book (“All of new dyeing methods” p136-150 (Iyaku Shuppansha)). After staining, the cells were sequentially immersed in 70, 80, and 100% ethanol for five minutes, respectively, to remove water, sealed in an Aquatex (produced by Merck Corp.), and then subjected to the observation of the staining image with an optical microscope. The results are shown in FIG. 1, and FIG. 2. From the results, it was clarified that all of HAST, TGF-α, EGF, and HB-EGF promoted the production of the mucus glycoprotein.

| Culture conditions | AB-PAS color intensity |
|---|---|
| PBS addition | — |
| EGF addition | ++++++ |
| HB-EGF addition | ++++++ |
| TGF-α addition | ++++++ |
| PBS addition | — |
| HAST addition | ++++ |
| HB-EGF addition | ++++++ |
| TGF-α addition | ++++++ |

(2) Mucus Glycoprotein Synthesis Due to HAST and Examination of HAST Activation-Inhibiting Effect

1) Cell Preparation

NCI-H292 cells (purchased from ATCC) were suspended in 10% FCS-containing RPMI-1640 (produced by Gibco Corp.), sowed on a Lab-Tek 8 well chamber slide (produced by Nunc Corp.) in a density of 6.0×10⁴ cells/cm², cultured for 72 hours, subjected to the replacement of the culture solution by RPMI-1640 containing 0.1% of BSA, and further cultured for 24 hours.

2) Drug Treatment

On the day of the treatment, the culture solution of each well was replaced by RPMI-1640 (containing 0.1% BSA) containing PBS alone, PBS and leupeptin (100 μM, produced by SIGMA Corp.), HAST alone (300 mU/mL), HAST (300 mU/mL) and leupeptin (100 μM), or HAST thermally denatured at 99° C. for 10 minutes. Thus, the cells were subjected to the start of the treatment, and further incubated at 37° C. for 24 hours.

3) AB-PAS Staining

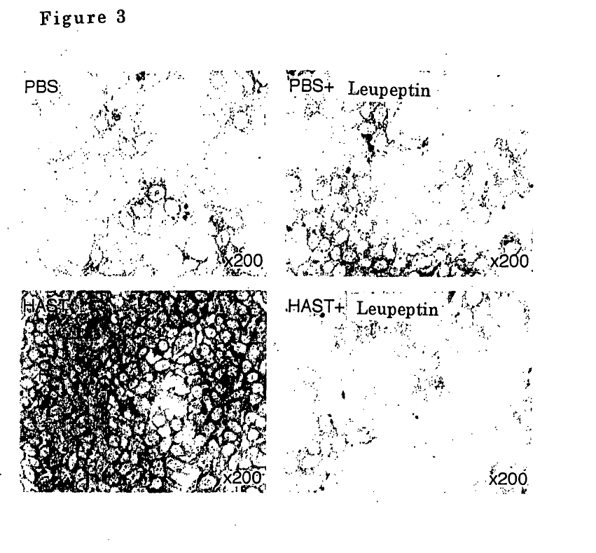

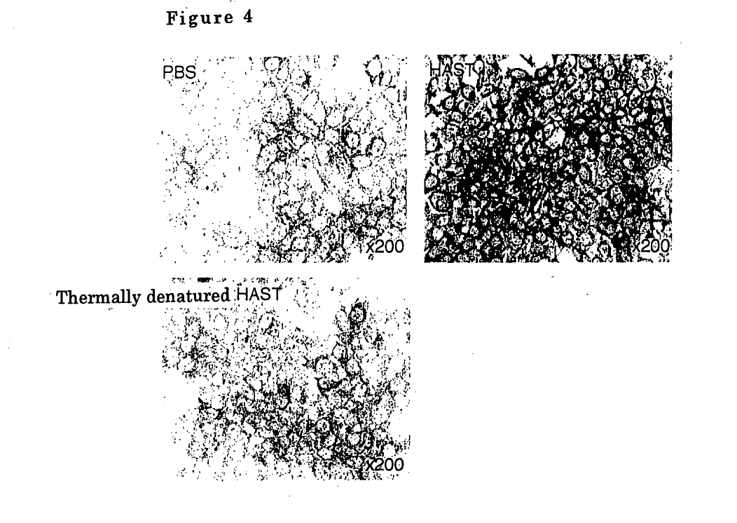

After the prescribed 24 hours treatment, the culture supernatant was removed. The residue was once washed with PBS, mixed with 4% paraformaldehyde/PBS, and then allowed to stand at room temperature for one hour to immobilize the cells. The immobilized cells were twice washed with Milli Q water and then staining with AB-PAS. The staining was carried out according to a book (“All of new dyeing methods” p136-150 (Iyaku Shuppansha)). After staining, the cells were sequentially immersed in 70, 80, and 100% ethanol for five minutes, respectively, to remove water, sealed in an Aquatex (produced by Merck Corp.), and then subjected to the observation of the staining image with an optical microscope. The results are shown in the next tables and FIGS. 3 and 4. From the results, it was clarified that the mucus glycoprotein production promotion caused by HAST could be controlled by inhibiting the activity of HAST with the protease inhibitor or by the thermal treatment.

| Culture conditions | AB-PAS color intensity |
|---|---|
| PBS addition | — |
| PBS/leupeptin addition | — |
| HAST addition | ++++++ |
| HAST/leupeptin addition | — |
| PBS addition | — |
| HAST addition | ++++++ |
| Thermally denaturated HAST addition | — |

### Example 14

### Analysis of Muc5AC mRNA Expression Quantity Due to Addition of AST by Real Time PCR Method

(1) Examination of Concentration-Dependent Muc5AC Production Increase Inhibition of Inhibitor

1) Cells

NCI-H292 cells (purchased from ATCC) were suspended in 10% FCS-containing RPMI-1640 (produced by Gibco Corp.), sowed on a 96 well plate (produced by Falcon Corp.) in a density of 6.0×10⁴ cells/cm², cultured for 72 hours, subjected to the replacement of the culture solution by RPMI-1640 containing 0.1% of BSA, and further cultured for 24 hours.

2) Drug Treatment

Before added to the cells, leupeptin in various concentrations was reacted with 300 mU/mL of HAST on ice for 30 minutes, and further heated at 37° C. Then, the culture solution in the wells was wholly exchanged by the culture medium containing the HAST or leupeptin to start the treatment, and the treatment was carried out for 24 hours. HAST (300 mU/mL) thermally treated at 99° C. for 10 minutes as a control, EGF alone (1 ng/mL), and a combination of EGF (5 ng/mL) with leupeptin (10⁻⁵ M) were also similarly treated. The number of the samples was 3.

3) RNA Extraction

The RNA was extracted with RNeasy Mini Kit and RNase-free DNase of QIAGEN Corp. for each well. 30 μL of the RNA solutions were obtained, respectively.

4) cDNA Synthesis

OmniScript-RT of QIAGEN Corp. was used. A cDNA synthesis reaction was carried out with a random primer and 0.25 μg of RNA to obtain 20 μL of the cDNA solution. A part of the cDNA solution was diluted with water 5 times to obtain a sample for TaqMan Assay.

5) TaqMan Assay

An EGF treatment sample was continuously diluted five times to prepare a standard. PCR reactions were carried out with cDNA diluted five times and the standard as templates, respectively. Commercially available primers and a commercially available probe (produced by ABI Corp.) were used for β-actin. Further, 5′-TCAACGGAGACTGCGAGTACAC-3′ as a forward primer, 5′-TCTTGATGGCCTTGGAGCA-3′ as a reverse primer, and 5′-ACTCCTTTCGTGTTGTCACCGAGAACGTC-3′ as a probe were used for Muc5AC. The primers and the probe were designed with Primer Express Ver 1.0. The primers were subcontracted and synthesized by Pharmacia Corp. The probe was also subcontracted and synthesized by ABI Corp. The PCR was carried out with TaqMan Universal PCR Mix (produced by ABI Corp.) and GeneAmp 5700 according to a procedure manual. The amplification reaction was carried out at 50° C. for two minutes, at 95° C. for 10 minutes, further at 95° C. for 15 seconds and at 60° C. for one minute totally in 40 cycles.

6) Analysis

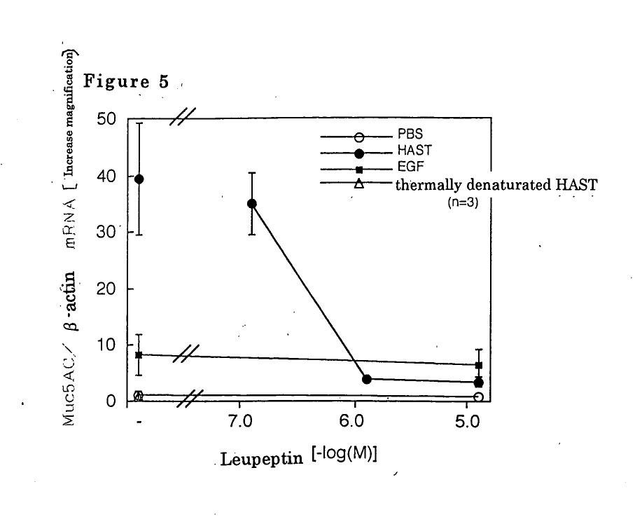

For both the Muc5AC and the β-actin, the expression quantities of the samples were relatively compared from the obtained Ct values and the standard straight line. Further, the values of the Muc5AC were divided by the values of the β-actin to correct the values. Finally, the values of the treated samples were divided by the average value of the untreated group to show the data as the increase magnifications, respectively. The results are shown in FIG. 5.

From the results, it was shown that the Muc5AC gene expression promotion caused by the HAST could be controlled by inhibiting the activity of the HAST with the protease inhibitor or by the thermal treatment.

(2) Examination of Muc5AC Production Increase Inhibition of Anti-EGF-R Antibody

1) Cells

NCI-H292 cells (purchased from ATCC) were suspended in 10% FCS-containing RPMI-1640 (produced by Gibco Corp.), sowed on a 96 well plate (produced by Falcon Corp.) in a density of 6.0×10⁴ cells/cm², cultured for 72 hours, subjected to the replacement of the culture solution by RPMI-1640 containing 0.1% of BSA, and further cultured for 24 hours.

2) Drug Treatment

\mathring{1} EGFR-neutralizing antibody treatment: the cultured product was preliminarily treated with 10 μg/mL concentration of an EGFR-neutralizing antibody (clone LA1; produced by Transduction Corp.) or 10 μg/mL concentration of normal mouse IgG1 (produced by R & D Corp.) at 37° C. for 30 minutes, and the culture medium was then wholly exchanged by RPMI-1640 (containing 0.1% of BSA) containing normal mouse IgG1 and HAST (300 mU/mL), the EGFR-neutralizing antibody and HAST (300 mU/mL), normal mouse IgG1 and EGF (1 ng/mL), or the EGFR-neutralizing antibody and EGF (1 ng/mL) to start the treatment. The treatment was further carried out for 24 hours. The number of samples was 3.

\mathring{2} Leupeptin treatment: HAST (300 mU/mL) and EGF (1 ng/mL) were preliminarily treated with leupeptin (10-4 M) on ice for 30 minutes, heated at 37° C., and then wholly exchanged by the culture medium in the wells to start the treatment. The treatment was further carried out for 24 hours. The number of the samples was 3.

3) RNA Extraction

The RNA was extracted with RNeasy Mini Kit and RNase-free DNase of QIAGEN Corp. for each well. 30 μL of the RNA solutions were obtained, respectively.

4) cDNA Synthesis

OmniScript-RT of QIAGEN Corp. was used. A cDNA synthesis reaction was carried out with a random primer and 0.25 μg of the RNA to obtain 20 μL of the cDNA solution. A part of the cDNA solution was diluted with water 5 times to obtain a sample for TaqMan Assay.

5) TaqMan Assay

An EGF treatment sample was continuously diluted five times to prepare a standard. PCR reactions were carried out with cDNA diluted five times and the standard as templates, respectively. Commercially available primers and a commercially available probe (produced by ABI Corp.) were used for β-actin. Further, 5′-TCAACGGAGACTGCGAGTACAC-3′ as a forward primer, 5′-TCTTGATGGCCTTGGAGCA-3′ as a reverse primer, and 5′-ACTCCTTTCGTGTTGTCACCGAGAACGTC-3′ as a probe were used for Muc5AC. The primers and the probe were designed with Primer Express Ver 1.0. The primers were subcontracted and synthesized by Pharmacia Corp. The probe was also subcontracted and synthesized by ABI Corp. The PCR was carried out with TaqMan Universal PCR Mix (produced by ABI Corp.) and GeneAmp 5700 according to a procedure manual. The amplification reaction was carried out at 50° C. for two minutes, at 95° C. for 10 minutes, further at 95° C. for 15 seconds and at 60° C. for one minute totally in 40 cycles.

6) Analysis

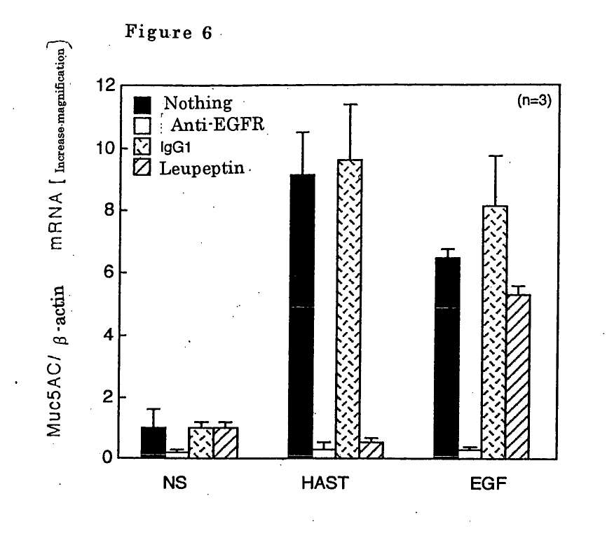

For both the Muc5AC and the β-actin, the expression quantities of the samples were relatively compared from the obtained Ct values and the standard straight line. Further, the values of the Muc5AC were divided by the values of the β-actin to correct the values. Finally, the values of the treated samples were divided by the average value of the untreated group to show the data as the increased magnifications, respectively. The results are shown in FIG. 6.

From the results, it was shown that the Muc5AC gene expression promotion caused by the HAST could be controlled by inhibiting the activity of the HAST with the protease inhibitor or by the thermal treatment. Further, it was shown that the HAST promoted the Muc5AC gene expression through the EGFR.

(3) Comparison with Trypsin, Esterase, Tryptase

1) Cells

NCI-H292 cells (purchased from ATCC) were suspended in 10% FCS-containing RPMI-1640 (produced by Gibco Corp.), sowed on a 96 well plate (produced by Falcon Corp.) in a density of 6.0×10⁴ cells/cm², cultured for 72 hours, subjected to the replacement of the culture solution by RPMI-1640 containing 0.1% of BSA, and further cultured for 24 hours.

2) Drug Treatment

On the day of the treatment, the culture solution of each well was replaced by RPMI-1640 (containing 0.1% BSA) containing HAST (0.3 mU/mL), esterase (0.1, 1, 10 U/mL), trypsin (0.1, 1, 10 U/mL) and tryptase (0.1, 1, 10 U/mL), and the treatment was carried out for 24 hour.

3) RNA Extraction

The RNA was extracted with RNeasy Mini Kit and RNase-free DNase of QIAGEN Corp. for each well. 30 μL of the RNA solutions were obtained, respectively.

4) cDNA Synthesis

OmniScript-RT of QIAGEN Corp. was used. A cDNA synthesis reaction was carried out with a random primer and 0.25 μg of the RNA to obtain 20 μL of the cDNA solution. A part of the cDNA solution was diluted with water 5 times to obtain a sample for TaqMan Assay.

5) TaqMan Assay

An EGF treatment sample was continuously diluted five times to prepare a standard. PCR reactions were carried out with cDNA diluted five times and the standard as templates, respectively. Commercially available primers and a commercially available probe (produced by ABI Corp.) were used for β-actin. Further, 5′-TCAACGGAGACTGCGAGTACAC-3′ as a forward primer, 5′-TCTTGATGGCCTTGGAGCA-3′ as a reverse primer, and 5′-ACTCCTTTCGTGTTGTCACCGAGAACGTC-3′ as a probe were used for Muc5AC. The primers and the probe were designed with Primer Express Ver 1.0. The primers were subcontracted and synthesized by Pharmacia Corp. The probe was also subcontracted and synthesized by ABI Corp. The PCR was carried out with TaqMan Universal PCR Mix (produced by ABI Corp.) and GeneAmp 5700 according to a procedure manual. The amplification reaction was carried out at 50° C. for two minutes, at 95° C. for 10 minutes, further at 95° C. for 15 seconds and at 60° C. for one minute totally in 40 cycles.

6) Analysis

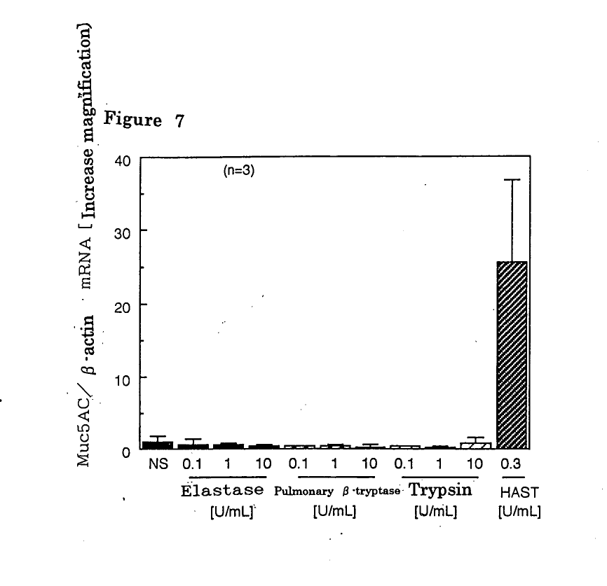

For both the Muc5AC and the β-actin, the expression quantities of the samples were relatively compared from the obtained Ct values and the standard straight line, respectively. Further, the values of the Muc5AC were divided by the values of the β-actin to correct the values. Finally, the values of the treated samples were divided by the average value of the untreated group to show the data as the increase magnifications, respectively. The results are shown in FIG. 7.

From the results, it was found that the Muc5AC gene expression was promoted by a mechanism which the trypsin, the tryptase and the esterase did not have.

### Example 15

### Cleavage of EGF-L (EGF and HB-EGF) on Surface of Cell Membrane with HAST

(1) Cleavage of EGF and HB-EGF Antigen on Surface of H292 Cell Membrane with HAST

1) Cells

NCI-H292 cells (purchased from ATCC) were cultured on a 10 cm dish in 10% FCS-containing RPMI-1640 until to reach a confluent state, subjected to the replacement of the culture medium by RPMI-1640 containing 0.1% of BSA, and further cultured for 24 hours. The cultured cells were washed with PBS, treated and floated in PBS containing 5 mM of EDTA at 37° C. for five minutes, and then suspended in 0.1%BSA-containing RPMI-1640 in a density of 1.0×10⁷ cells/mL.

2) Drug Treatment

The above-described cell suspension and 0.1% BSA-containing RPMI-1640 containing HAST in a double concentration (600 mU/mL) were mixed with each other in equivalent amounts, respectively, and reacted with each other at 37° C. for 30 minutes (final cell concentration: 5.0×10⁶ cells/mL, final HAST concentration: 300 mU/mL). The reaction solution was mixed with ice-cooled 1% FCS-containing PBS in a volume of 5 times that of the reaction solution to stop the reaction, and the treated solution was then supplied for a surface antigen analysis.

3) Surface Antigen Analysis

The cells subjected to the prescribed treatments were suspended in 1% FCS-containing PBS in a density of 1.0×10⁷ cells/mL. 100 μL of the prepared cell suspension was mixed with a primary antigen (EGF back ground dyeing: 1 μg of normal mouse IgG1 (produced by R & D Corp.), EGF dyeing: 1 μg of an anti-EGF mouse monoclonal antibody (produced by R & D Corp.), HB-EGF back ground dyeing: free from the primary antigen, HB-EGF dyeing: 2 μg of an anti-HB-EGF goat polyclonal antibody (produced by R & D Corp.)) and reacted on ice for 30 minutes. The reaction product was once washed with 5 mL of 1% FCS-containing PBS, mixed with 2 μg of a secondary antigen (EGF back ground dyeing and EGF dyeing: FITC conjugated F(ab′)₂ fragment to mouse IgG (produced by Dako Corp.), HB-EGF back ground dyeing and HB-EGF dyeing: Swine Anti-Goat Ig's Fluorescein Conjugate (produced by KPL Corp.)), and then reacted on ice for 30 minutes. The reaction solution was once washed with 5 mL of 1% FCS-containing PBS, and then mixed with Cell Fix (produced by Becton Dickinson Corp.) to immobilize the cells. The expression of the surface antigen was assayed with FACS Calibur (produced by Becton Dickinson Corp.) and analyzed with Cell Quest (produced by Becton Dickinson Corp.). Average fluorescence intensities were shown in the next table as the indicators of expression intensities.

From the results, it was found that the HAST cleaved proEGF and proHB-EGF on the surfaces of the cell membranes.

The Changes of EGF, HB-EGF Expression Intensities on the Surfaces of the Cell Membranes by the HAST Treatment

| | Back ground | PBS | HAST |
|---|---|---|---|
| EGF | 4.62 | 7.26 | 4.88 |
| HB-EGF | 4.5 | 8.72 | 6.94 |

(2) Examination of AST Activity-Inhibiting Effect

1) Cells

NCI-H292 cells (purchased from ATCC) were cultured on a 10 cm dish in 10% FCS-containing RPMI-1640 until to reach a confluent state, subjected to the replacement of the culture medium by RPMI-1640 containing 0.1% of BSA, and further cultured for 24 hours. The cultured cells were washed with PBS, treated and floated in PBS containing 5 mM of EDTA at 37° C. for five minutes, and then suspended in 0.1%BSA-containing RPMI-1640 in a density of 1.0×10⁷ cells/mL.

2) Drug Treatment

HAST (600 mU/mL) was reacted with leupeptin (10 μM) or BB1101 (5 μM) in 0.1% BSA-containing RPMI-1640 on ice for 30 minutes, and the reaction solution was mixed with the equivalent amount of a cell suspension preliminarily treated with leupeptin (10 μM) or BB1101 (5 μM) at 37° C. for 30 minutes, and further treated at 37° C. for 30 minutes (final HAST concentration: 300 mU/mL, final leupeptin concentration: 10 μM, final BB1101 concentration: 5 μM, final cell concentration: 5.0×10⁶ cells/mL). The reaction solution was mixed with ice-cooled 1% FCS-containing PBS in an amount of five times that of the reaction solution to stop the reaction, and then supplied for the surface antigen analysis.

3) Surface Antigen Analysis

The cells subjected to the prescribed treatments were suspended in 1% FCS-containing PBS in a density of 1.0×10⁷ cells/mL. 100 μL of the prepared cell suspension was mixed with a primary antigen (EGF back ground dyeing: 1 μg of normal mouse IgG1 (produced by R & D Corp.), EGF dyeing: 1 μg of an anti-EGF mouse monoclonal antibody (produced by R & D Corp.), HB-EGF back ground dyeing: free from the primary antigen, HB-EGF dyeing: 2 μg of an anti-HB-EGF goat polyclonal antibody (produced by R & D Corp.)) and reacted on ice for 30 minutes. The reaction product was once washed with 5 mL of 1% FCS-containing PBS, mixed with 2 μg of a secondary antigen (EGF back ground dyeing and EGF dyeing: PerCP conjugated F(ab′)₂ fragment to mouse IgG (produced by Becton Dickinson Corp.), or Swine Anti-Goat Ig's Fluorescein Conjugate), and then reacted on ice for 30 minutes. The reaction solution was once washed with 5 mL of 1% FCS-containing PBS, and then mixed with Cell Fix (produced by Becton Dickinson Corp.) to immobilize the cells. The expression of the surface antigen was assayed with FACS Calibur (produced by Becton Dickinson Corp.) and analyzed with Cell Quest (produced by Becton Dickinson Corp.). Average fluorescence intensities were shown in the next table as the indicators of expression intensities.

From the results, it was found that the HAST-caused cleavage of proEGF and proHB-EGF on the surfaces of the cell membranes was inhibited by inhibiting the activity of the HAST with the protease inhibitor.

The Changes of EGF, HB-EGF Expression Intensities on the Surfaces of the Cell Membranes by the HAST Treatment

| | Back | | | HAST + | HAST + |
|---|---|---|---|---|---|
| | ground | PBS | HAST | leupeptin | BB01 |
| EGF | 8.44 | 10.79 | 8.33 | 10.18 | 7.49 |
| HB-EGF | 5.58 | 8.61 | 6.26 | 7.3 | 6.65 |

(3) Evaluation of EGF-L (human HB-EGF) Excision Due to HAST

1) Preparation of Human HB-EGF Animal Cell Expression Plasmid

A cDNA was synthesized with SuperScript™ Preamplification System for First Strand cDNA Synthesis Kit (produced by GIBCO-BRL Corp.) using 2.5% g of human airway PolyA+RNA (produced by Clontech Corp.) and oligo(dT)₁₂₋₁₈ according to an appended protocol. A cDNA fragment encoding a Jaxtamemrane Domain-containing domain was obtained from the signal peptide of the N-terminal of human HB-EGF with the prepared human airway cDNA as a template by an amplification using PyorobestPCR (produced by Takara Shuzo Co.). On the other hand, human placenta alkali phosphatase cDNA was obtained from pSEAO2-Control vector (produced by Clontech Corp.) by an amplification with the Pyorobest PCR.

A DNA fragment: HBEGF (Δ TM)-ALP (SEQ ID NO: 44) obtained by adding human placenta alkali phosphatase to the C-terminal of Jaxtamemrane Domain was prepared by ligating a DNA fragment encoding a domain containing Jaxtamembrane Domain from the signal peptide of the N-terminal of human HB-EGF to a DNA fragment encoding human placenta alkali phosphatase by a PCR method.

A human HBEGF-expressing plasmid: pEF9-HBEGF (Δ TM)-ALP was prepared by inserting the above-described HBEGF into the multi-cloning site of the pEF9 vector prepared in Example 12 (1).

2) Expression of HBEGF in Human Cultured Cells

293EBNA cell strain was sowed in a density of 9×10⁶ cells/10 cm dish, cultured overnight, and then transfected with the human HBEGF-expressing plasmid: pEF9-HBEGF (Δ TM)-ALP prepared in 1). 12 μg of the pEF9-HBEGF (Δ TM)-ALP was subjected to a lipofection with 75 μL of Lipofectamine 2000 (produced by GIBCO-BRL). The lipofection was carried out according to an appended manual. 72 hour later, the cells were peeled with a pipette, and then centrifuged at 3000 rpm at 4° C. for five minutes. The lysate was dissolved in 12 mL of M-PER (produced by PIERCE Corp.), subjected to an SDS-polyacrylamide gel electrophoresis under a non-reducing condition, and then blotted on a nitrocellulose membrane (produced by BIO-RAD Corp.).

The product blotted on the nitrocellulose membrane was treated with 3% BSA-containing PBS at 4° C. overnight and then reacted with the anti-human HB-EGF antibody (produced by GENZYME Corp., an antibody recognizing an EGFR-binding site) diluted with 1% BSA-containing PBS in a concentration of 1 μg/mL, at room temperature for one hour. The nitrocellulose membrane was washed with 0.05% Tween 20-containing PBS three times, and then reacted with Swine Anti-Goat Ig's-ALP antibody (produced by BIOSOURCE Corp.) diluted with 1% BSA-containing PBS in a concentration of 1 μg/mL at room temperature for one hour. The nitrocellulose membrane was washed with 0.05% Tween 20-containing PBS three times, further once washed with TBS, and then reacted with NBT (nitroblue tetrazolium chloride)/BCIP (5-bromo-4-chloro-3-indolylphosphate p-toluidine salt, produced by PIERCE Corp.) at room temperature for five minutes to develop the color.

3) Activity of Cleavage of HBEGF (Δ TM)-ALP Due to HAST

100 μL of the lysate solubilized with the M-PER was mixed with 10 μL of a recombinant HAST (1.56 U/mL) and incubated at 37° C. overnight. The sample was subjected to the western blotting treatment similarly as described above. Consequently, the following facts were identified in the HAST-added sample. A band showing reactivity with the anti-human HB-EGF anti-body (an antibody recognizing an EGFR-binding site) was shifted to a low molecular weight. A mature type having the EGF domain of the HB-EGF was cut out. The band was shifted to a position of about 12 kD.

### Example 16

### Analysis of Tyrosine Phosphorylation of EGFR Due to HAST

1) Cells

NCI-H292 cells (purchased from ATCC) were suspended in 10% FCS-containing RPMI-1640 (produced by Gibco Corp.) and then sowed on a 12 well plate (made by Falcon Corp.) in a density of 6.0×10⁴ cells/cm². The cells were cultured for 72 hours, subjected to the replacement of the culture medium by 0.1% BSA-containing RPMI-1640, and further cultured for 24 hours.

2) Drug Treatment

On the day of the treatment, the culture solution of the wells was replaced by RPMI-1640 (containing 0.1% BSA) containing HAST (0.3 U/mL), EGF (1 μg/mL), and the treatments were carried out for 30, 60, 120, 180, 240 minutes, respectively.

3) Preparation of Cell Lysate

The cells treated with the HAST or the EGF for the prescribed time were twice washed with ice-cooled PBS, mixed with 500 μL of an ice-cooled Lysis buffer solution (50 mM Tris-HCl, pH 7.4, 5 mM EDTA, 1% Triton X-100 (Sigma), 150 mM NaCl, 10% glycerol, 1% Protease inhibitor Cocktail (Sigma), 1% Phosphatase Inhibitor Cocktail (Sigma)), allowed to stand on ice for 15 minutes, and then recovered in a micro tube. The recovered product in the micro tube was centrifuged at 15,000 rpm at 4° C. for 10 minutes, and the supernatant was recovered as a sample. The protein concentration of the sample was assayed with BioRad Protein Assay (produced by Bio Rad Corp.), and properly diluted with the Lysis buffer solution so that the protein concentrations in all the samples become 1.48 mg/mL. Subsequently, 406 μL of the diluted solution was used for the following immunoprecipitation.

4) Immunoprecipitation

The above-described prepared cell lysate (1.48 mg/mL, 406 μL) was mixed with 8 μL of agarose conjugated mouse IgG1 (produced by Santa Cruz Corp.) and 20 μL of Protein G Plus Agarose (produced by Oncogene Corp.), gently stirred at 4° C. for two hours, and then centrifuged at 3,000 rpm at 4° C. for one minute. The supernatant was recovered, mixed with 4 μg of an anti-EGFR antibody (Clone LA1, produced by Transduction Corp.), gently stirred at 4° C. for one hour, further mixed with 20 μL of Protein G Plus Agarose (produced by Oncogene Corp.), gently stirred at 4° C. overnight, and then centrifuged at 3,000 rpm at 4° C. for one minute. After the supernatant was removed, the precipitates were washed with 1 mL of an ice-cooled washing buffer solution (10 mM Tris-HCl, pH 7.4, 100 mM NaCl, 0.05% Tween-20 (BioRad) four times, further once washed with a washing buffer solution not containing Tween-20, and then centrifuged at 3,000 rpm at 4° C. for one minute. The supernatant was removed, and the precipitates were mixed with 25 μL of a SDS sample buffer solution (125 mM Tris-HCl, pH 6.8, 4% SDS, 10% glycerol, 0.04% bromophenol blue, 4% 2-mercaptoethanol), heated at 99° C. for five minutes, and then centrifuged at 3,000 rpm at room temperature for one minute. The supernatant was recovered and used as a sample for a western blotting treatment.

5) Western Blotting

The proteins separated by the SDS-polyacrylamide gel electrophoresis were transferred to a nylon membrane (Immobilon PVDF membrane, produced by Millipore Corp.) with a semi-dry transfer (produced by Ato Corp.). The transferred proteins were blotted in a blotting buffer solution (1% BSA (produced by Boehringer Mannheim GmbH)-containing washing buffer solution) at 4° C. overnight. Subsequently, the blotted proteins were reacted with a HRP-anti-phosphotyrosine antibody diluted with a blocking buffer solution 2,500 times, at room temperature for one hour. The membrane was washed with a washing buffer solution six times, and then subjected to the development of color.

6) Color Development, Detection

ECLplus (produced by Amasharm Pharmacia Biotech Corp.) was used. After washed, the membrane was immersed in an. ECL color-developing solution for five minutes, subjected to the removal of the excessive color-developing solution from the membrane, sealed in Saran Wrap, exposed to an X-ray film for about 2 to 5 minutes, developed, and then subjected to the analysis of the bands of the tyrosine phosphorylated proteins.

7) Reprobing

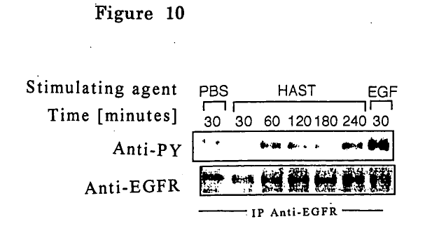

After exposed to the X-ray film, the membrane was washed with a washing buffer solution for five minutes, three times. The membrane was shaken in Restore reagent (produced by PIERCE Corp.) at 37° C. for 15 minutes to remove the antibody. The membrane was washed with a washing buffer solution for five minutes, three times, subjected to the development of the color by the above-described method with ECLplus, and then exposed to an X-ray film for 10 minutes. After it was identified that any band was not left in the developed X-ray film, the membrane was washed with a washing buffer solution for five minutes, three times, further immersed in a blocking buffer solution, and allowed to stand at 4° C. overnight to block. The blocked product was then reacted with an anti-EGFR antibody (Clone 13, produced by Transduction Corp.) diluted with a blocking buffer solution in a concentration of 0.1 μg/mL, at room temperature for one hour. The product was washed with a washing buffer solution six times, then reacted with an HRP-labeled anti-mouse IgG antibody (produced by Amersham Corp.) diluted with a blocking buffer solution 6,000 times, at room temperature for one hour, washed with a washing buffer solution six times, and then subjected to the color development using ECL plus by the above-described method. The color-developed band of EGFR was detected. The results are shown in FIG. 10.

Thus, it was clarified that HAST activated EGFR by the promotion of the tyrosine phosphorylation of EGFR.

### Example 17

### Acquisition of anti-AST Polyclonal Antibody

(1) Acquisition of Anti-Peptide Antibody

A 20 moiety peptide (SEQ ID NO: 25) ranged from isoleucine at the 187^{th} position of human AST to a moiety at the 205^{th} position and having cysteine at the N-terminal was chemically synthesized with a peptide synthesizer (Applide Biosystems Model 431A). The synthesized peptide was dissolved (10 mg/mL) in a 10 mM phosphate buffer solution (pH 7.5) and then incubated with 10 mg of maleimide-activated hemocyanin (produced by Boehringer Mannheim Biochemica Corp) at 25° C. for two hours. The reaction solution was dialyzed with a 10 mM phosphate buffer solution (pH 7.5). The hemocyanin-bound peptide was subcutaneously administered in a rabbit (0.5 mg/administration). The administration was repeated six times at two week intervals. The whole blood was collected, and the serum was prepared. The serum was purified with a sepharose 4B column (made by Pharmacia Corp.) to obtain the anti-AST polyclonal antibody (anti-N19 PAb).

(2) Acquisition of Anti-Recombinant AST Antibody

The recombinant AST (40 μg/administration) prepared in the Example 1 and Freund's complete adjuvant (produced by BACTO Corp., 1:1) were subcutaneously administered into a rabbit at two week intervals. After the treatment was carried out four times, the whole blood was collected to obtain an antiserum. IgG was purified from the serum with protein A sepharose 4B column (made by Pharmacia Corp.) according to a conventional method to obtain the anti-AST polyclonal antibody (anti-rAST PAb).

### Example 18

### Preparation of Anti-AST Monoclonal Antibody

(1) Immunization of Mouse with Recombinant AST

The recombinant AST prepared in Example 1 and Freund's complete adjuvant (produced by BACTO Corp., 1:1) were intraperitoneally administered (10 to 20 μg/administration/head) into a Balb/c mouse (7 week-old, male) at two week intervals. After the treatment was carried out four times, the fifth administration was carried out by intravenously administering 50 μg of a recombinant AST solution was administered three days before the day of cell fusion. Blood was collected from the tail vein of the mouse, incubated at 37° C. for 30 minutes, and then centrifuged at 3,000 rpm for 10 minutes to recover the serum. The anti-AST antibody titer in the serum was assayed by ELISA. Hereinafter, the method will be described.

50 μL of the recombinant AST diluted with 0.05M Na₂CO₃ buffer solution (pH 10.5) in a concentration of 1 μg/mL was added to a 96 hole LISA plate (Falcon 3912, Becton Dickinson Corp.) and reacted at 4° C. overnight. The reaction product was washed with PBS containing 0.05% Tween 20 three times, and then treated with PBS containing 3% bovine serum albumin (BSA) at room temperature for one hour. The treated product was washed with PBS containing 0.05% Tween 20 three times, mixed with 50 μL of a specimen, and reacted at room temperature for one hour. The reaction product was washed with PBS containing 0.05% Tween 20 three times, mixed with 50 μL of an alkali phosphatase-bound goat anti-mouse IgG antibody (ZYMED Corp.) diluted with 3% BSA-containing PBS 1,000 times, and reacted at room temperature for one hour. The reaction product was washed with PBS containing 0.05% Tween 20 three times, reacted with PNPP (p-nitrophenylphosphate, Wako Pure Chemical Industries, Ltd.) at room temperature for one hour, and then subjected to the assay of absorbance at 405 nm.

(2) Preparation of Hybridoma by Cell Fusion

A mouse was killed just before the cell fusion, and the spleen cells of the killed mouse were homogenized in PBS. The residues were filtered off with a nylon mesh and then once subjected to a centrifugal washing treatment using PBS. The obtained spleen cells were fused with mouse myeloma cells (P3X63Ag8U. 1) according to a conventional method (Kohler, Milstein; Nature, 256, 495 to 497, 1975).

Namely, 5×10⁷ spleen cells and 5×10⁶ mouse myeloma cells P3×63 Ag8U. 1(P3U1) were washed with RPMI 1640 culture medium and then centrifuged at 1,500 rpm for five minutes to obtain the cell pellet. 1 mL of the cell pellet was gradually mixed with a 35% polyethylene glycol solution (5.75 mL of RPMI 1640 culture medium +3.5 mL of polyethylene glycol +0.75 mL of dimethylsulfoxide) for two minutes to gently float the cells. The mixture was gradually mixed with 1 mL of RPMI 1640 culture medium for two minutes and further with 2 mL of RPMI 1640 culture medium for two minutes. The mixture was gradually mixed with 4 mL of GIT-HAT culture medium (GIT culture medium containing 95 μM hypoxanthine, 0.4 μM aminopterin, 1.6 μM thymidine, and 5% FCS) for two minutes, further mixed with 8 mL of GIT-HAT culture medium for two minutes, and then incubated at 37° C. for 30 minutes. The incubated product was dispensed into a 96 well flat bottom plate in whose each well about 10⁴ mouse peritoneal exudate cells were sowed, and then cultured in the presence of 5% CO₂ at 37° C.

One week later, the culture product was subjected to the exchange of a half of the culture medium by GIT-HT culture medium (a culture medium obtained by removing aminopterin from GIT-HAT culture medium), and further cultured in the presence of 5% CO₂ at 37° C. for about one week to obtain the several hybridoma colonies per well.

(3) Screening of Hybridoma

Two week later, the screening of the hybridoma was carried out with a plate coated with the recombinant AST. The 50 mM Na₂CO₃ buffer solution (1 μg/mL, pH 10.5) of the recombinant AST was dispensed into a 96 well plate (Falcon Corp., produced from PVC) in a volume of 50 μL per well, and then allowed to stand at 4° C. overnight. The product was washed and then blocked with 200 μL/well of 3% BSA/PBS at 37° C. for one hour. The product was again washed, mixed with 50 μL/well of the culture supernatant, allowed to stand at room temperature for one hour, and then washed with 0.05% Tween/PBS three times.

Subsequently, the product was mixed with 50 μl/well of goat anti-mouse IgG-alkali phosphatase conjugate (Tago Corp.) diluted with PBS containing 3% BSA and 0.2% skim milk 2,000 times, left a room temperature for one hour, again washed, mixed with 100 μL/well of the 1 mg/mL solution of disodium p-nitrophenylphosphate (Wako Pure Chemical Industries) dissolved in a 1M diethanolamine buffer solution (pH 9.8) containing 0.25 mM magnesium chloride, and reacted at room temperature for 30 minutes. The absorbance of the product at 405 nm was examined with an ELISA reader instrument (Vmax, Molecular Davice Corp.) for 96 well plates, and the hybridoma secreting the monoclonal antibody bound to the recombinant AST was selected.

(4) Cloning of Hybridoma

The selected hybridoma was twice cloned by a limiting dilution method to establish the hybridoma. Concretely, mouse peritoneal exudate cells prepared in HT culture medium in a density of 106 cells/mL were dispensed into wells, respectively, and the hybridoma cells suspended in HT culture medium in a rate of 0.5 cell/well were sowed in the wells, and then cultured in the presence of 5% CO₂ at 37° C. for two weeks. The culture supernatant was screened by the above-described ELISA method, and a single colony was picked up to establish the cells.

(5) Culture of Hybridoma and Purification of Antibody

Six clones among twenty anti-AST antibody-producing hybridoma clones obtained by the above-described operations were fit to and cultured in an eRDF culture medium (Gibco BRL Corp.) which is a serum-free culture medium containing insulin, transferrin, ethanolamine and selenite. The culture supernatant was recovered. The antibody in the supernatant was purified with a protein G column by a conventional method.

(6) Sub-Typing of AST Monoclonal Antibody

Six purified anti-AST monoclonal antibody clones obtained by the operations of (5) were sub-typed with IsoStrip (an isotyping kit for mouse monoclonal antibodies, Boehringer Corp.) according to a protocol. Consequently, it was found that three clones and two clones were IgG1 and IgM, respectively.

### Example 19

### Western Blotting with Anti-AST Antibody

A recombinant AST or natural AST was fractionated by an SDS polyacrylamide gel electrophoresis under a reducing condition, and then subjected to a western blotting on a nitrocellulose membrane (BIO-RAD Corp.). The product was treated with 3% BSA-containing PBS at room temperature overnight and then reacted with the anti-AST antibody obtained in Example 17, 18 and diluted with the 3% BAS-containing PBS in a density of 10 μg/mL, at room temperature for one hour. The nitrocellulose membrane was washed with 1% BSA-containing PBS fix times, and then reacted with goat anti-human IgG antibody (TAGO Corp.) bound to an alkali phosphatase diluted with 3% BSA-containing PBS in a density of 25% g/mL, at room temperature for one hour. The nitrocellulose membrane was washed with 1% BSA-containing PBS six times and then reacted with NBT/BICP (PIERCE Corp.) at room temperature for 10 minutes to develop the color.

Thereby, it was shown that the anti-AST antibodies were bound to the recombinant AST, respectively. In the reactivity of the western blotting, the reactivity of anti-N19 PAb among the polyclonal antibodies was high, and the reactivity of anti-rAST PAb was low. Further, the anti-rAST PAb did not exhibit a cross reactivity with mast cell-originated tryptase and trypsin. Normal rabbit IgG was used as a negative control.

### Example 20

### Evaluation of AST-Inhibiting Activity

(1) Method for Assaying AST Activity

The definitions of the assaying method and the activity unit are as shown in the above-described Example 2.

(2) Evaluation of AST-Inhibiting Activity of Anti-AST Antibody

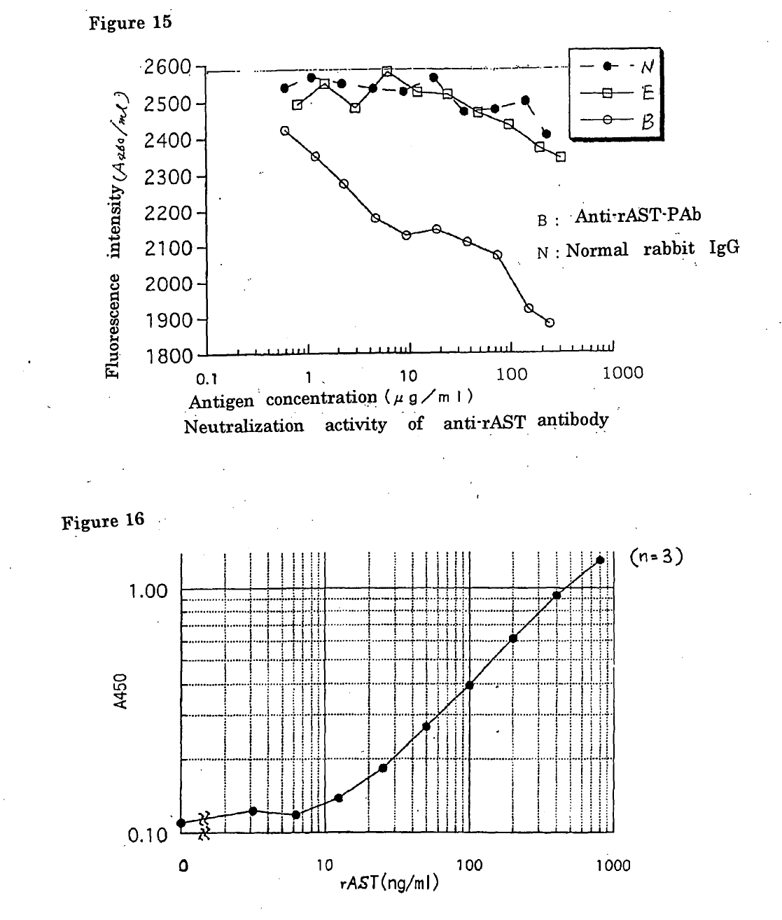

0.5 mL of a 50 mM Tris-HCl buffer solution (pH 8.6) containing 2 ng/mL of the recombinant AST and 0.01% of BSA was mixed with 50 μL of an anti-AST antibody-containing solution, and incubated at 37° C. for 30 minutes. The incubation product was mixed with 0.5 mL of a 50 mM Tris-HCl buffer solution (pH 8.6) containing 200 μM of a trypsin synthesis substrate: Boc-Phe-Ser-Arg-MCA, and incubated at 37° C. for one hour. The product was mixed with 1 mL of 30% acetic acid. The quantity of the produced AMC was assayed by a fluorometry (fluorescent light: 460 nm, and exciting light: 380 nm), and the enzymatic activity-inhibiting ability of the anti-AST antibody was evaluated. The results are shown in FIG. 15.

In the anti-AST monoclonal antibody obtained by immunizing the recombinant AST (insect cell-baculovirus system) obtained in Example, an activity-inhibiting effect of 27% was recognized in a concentration of 236 μg/mL.

(3) Evaluation of AST-Inhibiting Activity of Compound

0.05 mL of a 50 mM Tris-HCl buffer solution (pH 8.6) containing 2 ng/mL of the recombinant AST and 0.01% of Tween 20 was mixed with 2 μL of a DMSO solution containing nafamostat mesylate or gabexate mesylate as an evaluation target compound in various concentrations, and with 0.05 mL of a 50 mM Tris-HCl buffer solution (pH 8.6) containing 30 μM of a trypsin synthesis substrate: Boc-Phe-Ser-Arg-MCA, and incubated at 24° C. for 6 minutes. The quantities of the produced AMC were assayed by a fluorometry (fluorescent light: 460 nm, exciting light: 380 nm) with the passage of time, and the enzymatic activity-inhibiting ability of the compound was evaluated. The results are shown in the next table as IC 50 (molar concentration exhibiting the inhibiting activity of 50%).

| Compound name | IC 50 (μM) |
|---|---|
| Nafamostat mesylate | 0.23 |
| Gabexate mesylate | 0.14 |

### Example 21

### Construction of AST Assay System

(1) Construction of Sandwich ELISA System with Polyclonal Antibody

The following operations using the anti-AST polyclonal antibody (anti-rASTPAb) obtained in Example 17 were carried out to construct the sandwich ELISA system.

1) Preparation of Horse Radish•Peroxidase (HRP)-Labeled Antibody

\mathring{1} Preparation of Antibody (F(ab′)₂)

1 mL of the 2.0 mg/mL PBS solution of an antibody (IgG) was mixed with 100 μL of a 1M acetate buffer solution (pH 4.2) and 20 μL of the same buffer solution containing 40 μg of pepsin, and reacted at 37° C. for four hours. After the reaction is finished, the reaction solution was subjected to a separation treatment using a PBS-equilibrated Sephadex G 25 column (φ 2 cm×45 cm) to collect the antibody (F(ab′)₂).

\mathring{2} HRP Label of Antibody (F(ab′)₂)

2 mL of a solution (pH 7.4) of 1 mg/mL of the antibody (F(ab′)₂), 0.01M phosphate and 0.15M NaCl was mixed with 50 μL of the dimethylformamide solution (10 mg/mL) of N-(m-maleimide benzoic acid)-N-succinimide ester (MBS), and reacted at 25° C. for 30 minutes. The reaction solution was then subjected to a gel filtration treatment using a column filled with Sephadex G-25 in a 0.1M phosphate buffer solution (pH 6.0) to separate the maleimidized antibody from the unreacted MBS.

On the other hand, 2 mL of a 0.1M phosphate buffer solution (pH 6.5) containing 10 mg/mL of HRP was mixed with 120 μL of a dimethylformamide solution containing 60 mg/mL of S-acetylmercaptosuccinic anhydride, and reacted at 25° C. for two hours. The reaction solution was mixed 800 μL of a 0.1M Tris-HCl buffer solution (pH 7.0), 160 μL of 0.1M EDTA and 1.6 mL of 1M hydroxylamine, and reacted at 0° C. for four minutes. Subsequently, the reaction solution was charged in a collodion bag and then dialyzed with a 0.1M phosphate buffer solution (pH 6.0) and a 5 mM EDTA-containing solution at 4° C. for three days to obtain the thiolated HRP.

Subsequently, 2 mg of the maleimidized antibody was mixed with 4 mg of the thiolated HRP, and the mixture was concentrated with a collodion bag under cooling with ice until to give a protein concentration of 4 to 10 mg/mL, and then allowed to stand at 15 to 20° C. overnight. The solution was subjected to a gel filtration using a column filled with Ultrogel AcA44 (produced by Pharmacia LKB Corp.) to obtain the anti-AST (F(ab′)₂) HRP-labeled antibody.

2) Preparation of Antibody-Immobilized Plate

A 96 well plate (produced by Sumitomo Bakelite Co., MS-3796F/carboplate) was well washed, allowed to stand in the 20 μg/mL PBS solution of an antibody at 4° C. for a whole day and night, washed with PBS, and then allowed to stand in the 1% PBS solution of bovine serum albumin (BSA) at 4° C. for a whole day and night to carry out the post-coating treatment, thus obtaining the antibody-immobilized plate.

3) Construction of Assay System

100 μL of a 1% BSA-0.1% skim milk-containing 10 mM PBS (pH 7.2) solution (diluted specimen solution) containing a purified recombinant AST (standard substance) in a concentration of 0 to 1,600 ng/mL was added to the anti-AST antibody IgG-immobilized plate prepared in 2), and then incubated at 25° C. for four hours. Subsequently, the plate was washed with 0.05% Tween 20-containing 10 mM PBS (pH 7.2). 100 μL of the 1% BSA-containing 10 mM (pH 7.2) 100-fold diluted solution of the anti-AST (F(ab′)₂) HRP-labeled antibody (500 μg/mL) prepared in 1) was added to each well, and incubated at 25° C. for two hours. The wells were subjected to the suctional removal of the solutions in each well, and then washed with 0.05% Tween 20-containing 10 mM PBS (pH 7.2). 100 μL of a 0.1M phosphate/citrate buffer solution (pH 4.3) containing 0.02% of 3,3′,5,5′-tetramethylbenzidine hydrochloride and 2.5 mM of hydrogen peroxide was added to each well, and reacted at 25° C. for 30 minutes. 100 μL of a 0.5M sulfuric acid aqueous solution was added to each well as a reaction stopper to stop the enzyme reaction. The absorption intensity of the solution at a wavelength of 450 nm was assayed with a spectrophotometer, and the assayed absorption intensities were plotted in response to the standard substance concentrations of 0 to 1,600 ng/mL. The results are shown in FIG. 16.

4) Detection of AST in Intravital Component

\mathring{1} Detection of AST in Saliva

AST in human saliva. was assayed with the assay system in 3). The saliva collected from a human being was centrifuged at 15,000 rpm for 15 minutes, and the supernatant was used as an assay sample. Six samples were assayed in an undiluted liquid state or in a state diluted with a 1% BSA-0.1% slim milk-containing 10 mM PBS (pH 7.2) solution (specimen-diluting solution) sixteen times. Since the assayed values of the three samples were low, the values of the undiluted solutions were adopted, and for the other three samples, the values of the 16-fold diluted solutions were adopted as the AST concentrations of the saliva samples. The assay results are shown together with the AST. activities (trypsin substrate-degrading activities) in the samples in the next table.

AST (ng/mL)

| Saliva samples | ELISA | AST activity conversion * |
|---|---|---|
| KM 123 | 1616 | 3333 |
| KY 123 | 24 | 120 |
| KT 123 | 22 | 518 |
| HO 123 | 720 | 1772 |
| KY 112 | 25 | 208 |
| HO 112 | 784 | 2349 |

* Recombinant HAT conversion trypsin-like enzyme activity

\mathring{2} Detection of AST in Sputum

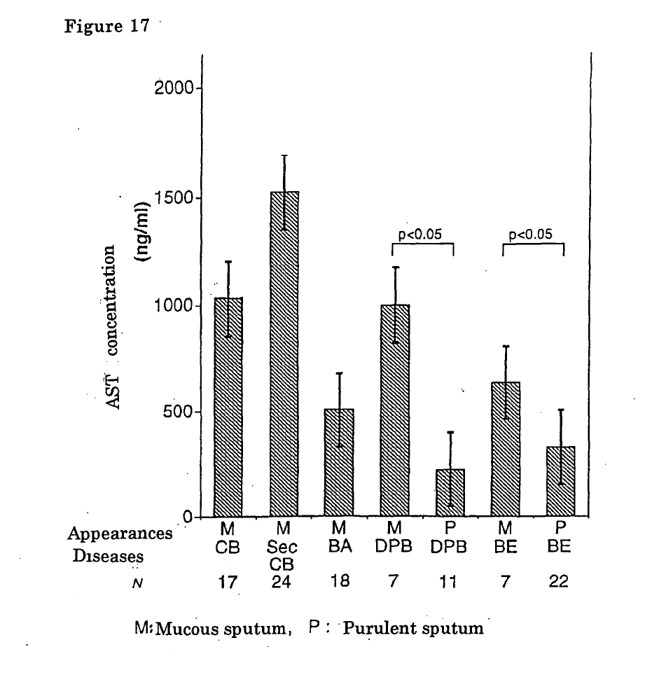

Sputum was collected from a chronic respiratory inflammation patient, diluted with a nine-fold volume of physiological salt solution, homogenized, and then centrifuged at 10,000×g for 10 minutes. The supernatant was used as an assay specimen. The specimen was diluted twenty times, and assayed. The results are shown in FIG. 17. It was found that the assay values of purulent sputa were lower than those of mucous sputa.

Herein, the mucous-mucos purulent sputa were collected from the following patients.

CB: chronic bronchitis

SecCB: secondary bronchitis

BA: bronchial asthma

BE: bronchiectasis

DPB: diffuse panbronchiolitis

Further, the purulent sputa were collected from the following patients.

BE: bronchiectasis

DPB: diffuse panbronchiolitis

(2) Construction of Sandwich ELISA System with Monoclonal Antibody and Polyclonal Antibody

1) Evaluation of Liquid Phase Antigenic Reactivity of Each Anti-AST Monoclonal Antibody

The evaluation of a binding property to the recombinant AST in a liquid phase was carried out with respect to three kinds of anti-AST monoclonal antibodies which were IgG1 type by a sub-typing.

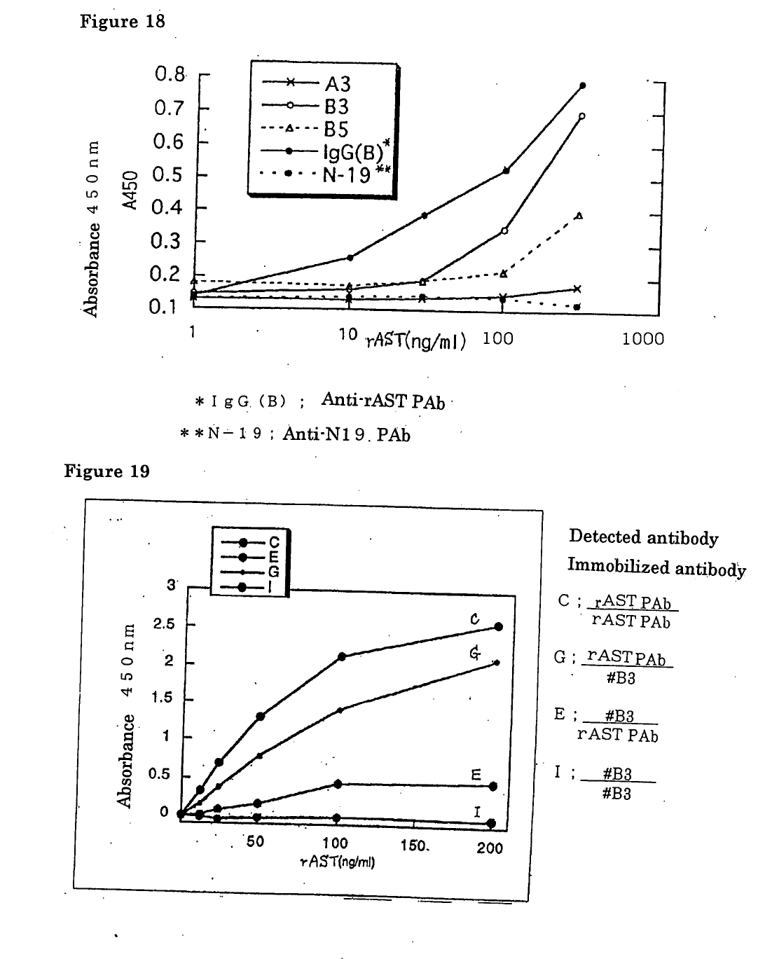

50 μL/well of a PBS solution (1 and 5 μg/mL) of each anti-AST monoclonal antibody was added to a 96 well plate, and allowed to stand at 4° C. overnight to immobilize the antibody. 100 μL of a 1% BSA-0.1% skim milk-containing 10 mM PBS (pH 7.2) solution (specimen-diluting solution) containing the purified recombinant AST (standard substance) in a range of 0 to 300 ng/mL was added to the prepared anti-AST monoclonal antibody-immobilized plate, and then incubated at 25° C. for four hours. Subsequently, the plate was washed with 0.05% Tween 20-containing 10 mM PBS (pH 7.2), subjected to the addition of 100 μL of a solution obtained by diluting the HRP-labeled antibody (500 μg/mL) prepared in (1) 1) with 1% BSA-containing 10 mM PBS (pH 7.2) 100 times to each well, and then incubated at 25° C. for two hours. Then, the solution was sucked off from each well, and the well was washed with 0.05% Tween 20-containing 10 mM PBS (pH 7.2). 100 μL of a 0.1M phosphate/citrate buffer solution (pH 4.3) containing 0.02% of 3,3′,5,5′-tetramethylbenzidine hydrochloride and 2.5 mM of hydrogen peroxide was added to each well. The reaction was carried out at 25° C. for 30 minutes, and 100 μL of a 0.5M aqueous sulfuric acid solution was added to each well to stop the enzymatic reaction. Subsequently, the absorption intensity of the solution at a wavelength of 450 nm was assayed with a spectrophotometer. The assayed absorption intensities were plotted in response to the standard substance concentrations of 0 to 300 ng/mL. The results are shown in FIG. 18.

2) Construction of Assay System

\mathring{1} Preparation of Biotinylated Antibody

The biotinylation of IgG was carried out with the # B3 anti-AST monoclonal antibody having the highest liquid phase antigenic reactivity in the evaluation results of 1) and with the anti-AST polyclonal antibody (anti-rAST PAb) obtained in Example 17.

1 mL of a 1 to 3 mg/mL IgG/PBS (−) solution was mixed with the equivalent volume of 0.2M NaHCO₃ (pH 8.0), further mixed with 10 mM of a biotinylating reagent having a molar concentration of 100 times, and then incubated at 37° C. for one hour. As the biotinylating reagent, NHS-LC-Biotin (produced by PIERCE Corp., #21335) dissolved in dimethylformamide in a concentration of 10 mM was used. The reaction solution was mixed with one-tenth amount of 1M monoethanolamine and further incubated at 37° C. for one hour. The reaction solution was diluted with PBS (−) into 2.5 mL, and then applied to a G-25 gel filtration column to allow to flow out the PBS (−), thus recovering the biotinylated IgG. The recovered biotinylated IgG was mixed with BSA in a final concentration of 3%, further mixed with NaN₃ in a final concentration of 0.1%, and then stored.

\mathring{2} Preparation of Antibody-Immobilized Plate

A 96 well plate (produced by Sumitomo Bakelite Co., MS-3796F/carboplate) was well washed, subjected to the addition of 100 μL of the 20 μg/mL PBS solution of an antibody (#B3 and anti-rAST PAb) to each well, and allowed to stand at 4° C. for a whole day and night. The treated plate was washed with PBS-0.05% Tween 20, subjected to the addition of 380 μL of a 1% bovine serum albumin PBS solution to each well, and then allowed to stand at 4° C. for a whole day and night to post-treat the wells, thus obtaining the antibody-immobilized plate.

\mathring{3} Construction of Assay System

50 μL of a 1% BSA-0.1% skim milk-containing 10 mM PBS (pH 7.2) solution (specimen-diluting solution) containing the purified recombinant AST (standard substance) in a range of 0 to 400 nm/mL, and 50 μL of a 1% BSA-0.1% skim milk-containing 10 mM PBS (pH 7.2) solution containing the biotinylated IgG prepared in \mathring{1} were added to the anti-AST IgG-immobilized plate prepared in \mathring{2}, and then allowed to stand at 4° C. overnight. The plate was washed, subjected to the addition of 100 μL/well of a 1% BSA-0.1% skim milk-containing 10 mM PBS (pH 7.2) solution obtained by diluting streptavidin-labeled peroxidase (produced by TAGO Corp., # SNN1004) 10,000 times to each well, incubated a 25° C. for one hour, subjected to the suctional removal of the solution in each well, washed with 0.05% Tween 20-containing 10 mM PBS (pH 7.2), and further subjected to the addition of 100 μL of a 0.1M phosphate/citrate buffer solution (pH 4.3) containing 0.02% of 3,3′,5,5′-tetramethylbenzidine hydrochloride and 2.5 mM of hydrogen peroxide to each well. The reaction was carried out at 25° C. for 15 minutes, and 100 μL of a 0.5M aqueous sulfuric acid solution was added to each well to stop the enzymatic reaction. Subsequently, the absorption intensity of the solution at a wavelength of 450 nm was assayed with a spectrophotometer. The assayed absorption intensities were plotted in response to the standard substance concentrations of 0 to 200 ng/mL. The results are shown in FIG. 19.

### INDUSTRIAL APPLICABILITY

The AST having the structure in which the whole propeptide moiety or a part thereof is bound to the trypsin-like protein moiety via the disulfide bond is provided by the present invention. The AST having said structure can be used to construct an inhibitor-screening system using the PAR activation, mucus production promotion, EGFR signal transduction system activation, or intracellular calcium influx promotion activity as an indicator, and thus screen an inhibitor, thereby obtaining an AST inhibitor and an inhibitory polypeptide (including inhibitory antibodies). It can be expected that the thus obtained AST inhibitor, the AST PAR activation inhibitor, the AST mucus production promotion inhibitor, the EGFR signal transduction system activation inhibitor, or the intracellular calcium influx promotion inhibitor is used as a therapeutic drug to control or modify a physiological action such as the wide sense airway inflammation actions, mucus secretion promotion action, airway remodeling action, mucociliary self defence system action, coagulation•fibrinogenolysis system action, cell proliferation action, cancer proliferation or metastasis action, or anti-virus infection action, to prevent disease crisis or to improve and treat the state of the disease.

In addition, on the basis of the sequence of the animal AST, such as mouse AST, of the present invention, it can be judged in what disease state model animal and at what time the AST participates in the state of a disease by assaying or controlling the protein expression or gene expression of the animal AST. Further, the therapeutic effects of the AST inhibitor and the inhibitory protein selected by utilization of the present invention can be judged, and the clinical doses of the AST inhibitor or the inhibitory polypeptide can also be estimated.

Further, the expression sites of tissues can be detected by immunohistochemistry using the monoclonal antibody and polyclonal antibody of the present invention.

Furthermore, the enzyme immunoassay system, diagnostic reagent or diagnostic kit using the monoclonal antibody and polyclonal antibody of the present invention enables the assay of the concentration of the AST in saliva, sputum, serum or the like, and is thereby useful for understanding and diagnosing the state of an AST-participating disease.

What is claimed is:

1. An airway-specific trypsin-like enzyme which is a protein comprising the whole amino acid sequence represented by SEQ ID NO: 1 or a part thereof and has a structure in which a propeptide moiety comprising the whole amino acid sequence from Met at the 1^{st} position to Arg at the 186^{th} position or a part thereof is bound to a trypsin-like protein moiety comprising a sequence of 232 amino acids from Ile at the 187^{th} position to Ile at the 418^{th} position via a disulfide bond.

2. A mammalian airway-specific trypsin-like enzyme which is a mammalian counterpart protein having a 66% or more homology with the airway-specific trypsin-like enzyme represented by SEQ ID NO: 1 and has a structure in which a moiety corresponding to said propeptide moiety is bound to a moiety corresponding to said trypsin-like protein moiety via a disulfide bond.

3. A method for detecting the airway-specific trypsin-like enzyme-inhibiting activity of a compound or a polypeptide to be assayed, comprising:
mixing an enzyme substrate with the compound or the polypeptide to be assayed;
incubating the mixture and the airway-specific trypsin-like enzyme according to claim 1 or 2 or a cell expressing said enzyme or a tissue expressing said enzyme under proper conditions to react said enzyme substrate; and
assaying the reaction product.

4. A method for detecting the inhibitory activity of a compound or a polypeptide to be assayed against protease-activated receptor signal transduction pathway activation due to an airway-specific trypsin-like enzyme, comprising:
mixing the airway-specific trypsin-like enzyme according to claim 1 or 2 or a cell expressing said enzyme or a tissue expressing said enzyme, the compound or the polypeptide to be assayed, and further a protease-activated receptor-expressing cell; and
using the protease-activated receptor signal transduction pathway activation as an indicator.

5. The detection method according to claim 4, wherein the protease-activated receptor is PAR2.

6. A method for evaluating the inhibitory activity of a compound or a polypeptide to be assayed against intracellular calcium influx promotion due to the airway-specific trypsin-like enzyme according to claim 1 or 2, by using an intracellular calcium concentration as an indicator.

7. A method for evaluating the inhibitory activity of a compound or a polypeptide to be assayed against an IL-8 release-promoting action due to the airway-specific trypsin-like enzyme according to claim 1 or 2, by using the release quantity or expression quantity of IL-8 or the transcription activity of the promoter as an indicator.

8. A method for detecting the inhibitory activity of a compound or a polypeptide to be assayed against mucus production promotion due to the airway-specific trypsin-like enzyme according to claim 1 or 2, comprising:
mixing the airway-specific trypsin-like enzyme or a cell expressing said enzyme or a tissue expressing said enzyme, the compound or the polypeptide to be assayed, and further a cell having a mucus-producing ability; and
using the mucus-producing ability as an indicator.

9. The detection method according to claim 8, wherein a method for assaying the mucus-producing ability is an AB-PAS staining method.

10. The detection method according to claim 8, wherein the indicator of the mucus-producing ability is the quantity of expressed Muc5AC.

11. The detection method according to claim 8, wherein the indicator of the mucus-producing ability is the quantity of released or produced EGFR-L.

12. The detection method according to claim 8, wherein the indicator of the mucus-producing ability is the activation of an EGF-R signal transduction pathway.

13. A method for detecting the inhibitory activity of a compound or a polypeptide to be assayed against EGFR pathway activation promotion due to the airway-specific trypsin-like enzyme according to claim 1 or 2, comprising:
mixing the airway-specific trypsin-like enzyme or a cell expressing said enzyme or a tissue expressing said enzyme, the compound or the polypeptide to be assayed, and further a cell having an EGFR signal transduction system; and
using the activation of the EGFR signal transduction system as an indicator.

14. A gene encoding the airway-specific trypsin-like enzyme according to claim 1 or 2 or a mRNA or cDNA originated from said gene.

15. An expression vector used for the airway-specific trypsin-like enzyme, which prepared by using the whole gene, mRNA, or cDNA according to claim 14 or a part thereof.

16. A cell transfected or transformed with the expression vector according to claim 15.

17. A method for purifying the airway-specific trypsin-like enzyme according to claim 1 or 2, comprising:
culturing the cell according to claim 16; and
purifying the enzyme from the culture solution.

18. The airway-specific trypsin-like enzyme according to claim 1 or 2, which purified by the method according to claim 17.

19. The method for detecting the expression quantity of the airway-specific trypsin-like enzyme gene corresponding to the gene, the mRNA or the cDNA according to claim 14, by using the whole information of the nucleic acids or a part thereof.

20. A method for screening a substance inhibiting the activity of the airway-specific trypsin-like enzyme according to claim 1 or 2 or inhibiting the activation of said enzyme or a method for judging a therapeutic effect thereof, comprising administering a compound or a polypeptide to be assayed to a mammal having a disease characterized by the activation excess or up-regulate of the airway-specific trypsin-like enzyme, and then judging the effect.

21. A method for screening a substance inhibiting the protease-activated receptor activation of the airway-specific trypsin-like enzyme according to claim 1 or 2, or a method for judging a therapeutic effect thereof, comprising:
administering a compound or a polypeptide to be assayed to a mammal having a disease characterized by protease-activated receptor activation excess or up-regulate due to the airway-specific trypsin-like enzyme; and
judging the effect.

22. A method for screening a substance inhibiting mucus production promotion due to the airway-specific trypsin-like enzyme according to claim 1 or 2, or a method for judging a therapeutic effect thereof, comprising:
administering a compound or a polypeptide to be assayed to a mammal having a disease characterized by the mucus production promotion or up-regulate due to the airway-specific trypsin-like enzyme; and
judging the effect.

23. A method for screening a substance inhibiting EGFR pathway activation promotion due to the airway-specific trypsin-like enzyme according to claim 1 or 2, or a method for judging a therapeutic effect thereof, comprising:
administering a compound or a polypeptide to be assayed to a mammal having a disease characterized by EGFR pathway activation promotion or up-regulate due to the airway-specific trypsin-like enzyme; and
judging the effect.

24. An antibody specifically binding to the airway-specific trypsin-like enzyme according to claim 1 or 2.

25. A monoclonal antibody specifically binding to the airway-specific trypsin-like enzyme according to claim 1 or 2.

26. A monoclonal antibody inhibiting the activity of the airway-specific trypsin-like enzyme according to claim 1 or 2 and/or inhibiting the activation thereof.

27. A method for detecting or assaying the airway-specific trypsin-like enzyme by using the antibody according to either of claim 24 to 26.


SEQUENCE LISTING (59 sequences)

SEQ ID NO: 1 - protein, 418 aa, Homo sapiens
MYRPARVTSTSRFLNPYVVCFIVVAGVVILAVTIALLVYFLAFDQKSYFYRSSFQLLNVEYNSQLNSPATQEYRTLSGRIESLITKTFKESNLRNQFIRAHVAKLRQDGSGVRADVVMKFQFTRNNNGASMKSRIESVLRQMLNNSGNLEINPSTEITSLTDQAAANWLINECGAGPDLITLSEQRILGGTEAEEGSWPWQVSLRLNNAHHCGGSLINNMWILTAAHCFRSNSNPRDWIATSGISTTFPKLRMRVRNILIHNNYKSATHENDIALVRLENSVTFTKDIHSVCLPAATQNIPPGSTAYVTGWGAQEYAGHTVPELRQGQVRIISNDVCNAPHSYNGAILSGMLCAGVPQGGVDACQGDSGGPLVQEDSRRLWFIVGIVSWGDQCGLPDKPGVYTRVTAYLDWIRQQTGI

SEQ ID NO: 2 - protein, 417 aa, Mus musculus
MYRPRPMLSPSRFFTPFAVAFVVIITVGLLAMMAGLLIHFLAFDKKAYFYHSSFQILNVEYTEALNSPATHEYRTLSERIEAMITDEFRGSSLKSEFIRTHVVKLRKEGTGVVADVVMKFRSSKRNNRKVMKTRIQSVLRRLSSSGNLEIAPSNEITSLTDQDTENVLTQECGARPDLITLSEERIIGGMQAEPGDWPWQVSLQLNNVHHCGGALISNMWVLTAAHCFKSYPNPQYWTATFGVSTMSPRLRVRVRAILAHDGYSSVTRDNDIAVVQLDRSVAFSRNIHRVCLPAATQNIIPGSVAYVTGWGSLTYGGNAVTNLRQGEVRIISSEECNTPAGYSGSVLPGMLCAGMRSGAVDACQGDSGGPLVQEDSRRLWFVVGIVSWGYQCGLPNKPGVYTRVTAYRNWIRQQTGI

SEQ ID NO: 3 - DNA, 1500 bp, Homo sapiens
  exon (62)..(1315)
gagtgggaatctcaaagcagttgagtaggcagaaaaaagaacctcttcattaaggattaaaatgtataggccagcacgtgtaacttcgacttcaagatttctgaatccatatgtagtatgtttcattgtcgtcgcaggggtagtgatcctggcagtcaccatagctctacttgtttactttttagcttttgatcaaaaatcttacttttataggagcagttttcaactcctaaatgttgaatataatagtcagttaaattcaccagctacacaggaatacaggactttgagtggaagaattgaatctctgattactaaaacattcaaagaatcaaatttaagaaatcagttcatcagagctcatgttgccaaactgaggcaagatggtagtggtgtgagagcggatgttgtcatgaaatttcaattcactagaaataacaatggagcatcaatgaaaagcagaattgagtctgttttacgacaaatgctgaataactctggaaacctggaaataaacccttcaactgagataacatcacttactgaccaggctgcagcaaattggcttattaatgaatgtggggccggtccagacctaataacattgtctgagcagagaatccttggaggcactgaggctgaggagggaagctggccgtggcaagtcagtctgcggctcaataatgcccaccactgtggaggcagcctgatcaataacatgtggatcctgacagcagctcactgcttcagaagcaactctaatcctcgtgactggattgccacgtctggtatttccacaacatttcctaaactaagaatgagagtaagaaatattttaattcataacaattataaatctgcaactcatgaaaatgacattgcacttgtgagacttgagaacagtgtcacctttaccaaagatatccatagtgtgtgtctcccagctgctacccagaatattccacctggctctactgcttatgtaacaggatggggcgctcaagaatatgctggccacacagttccagagctaaggcaaggacaggtcagaataataagtaatgatgtatgtaatgcaccacatagttataatggagccatcttgtctggaatgctgtgtgctggagtacctcaaggtggagtggacgcatgtcagggtgactctggtggcccactagtacaagaagactcacggcggctttggtttattgtggggatagtaagctggggagatcagtgtggcctgccggataagccaggagtgtatactcgagtgacagcctaccttgactggattaggcaacaaactgggatctagtgcaacaagtgcatccctgttgcaaagtctgtatgcaggtgtgcctgtcttaaattccaaagctttacatttcaactgaaaaagaaactagaaatgtcctaatttaacatcttgttacataaatatggtttaacaaacactgtttaacctttctttattattaaaggttttctattttctcc

SEQ ID NO: 4 - DNA, 1462 bp, Mus musculus
  exon (65)..(1315)
cgccgggcaggtcaaagcagctggacacacagaaacaaggacctcttcattattcaagagtaaaatgtataggccaagaccaatgctatcaccgtcaagattcttcactccctttgcagtagctttcgttgtcataataacggtagggctcctggccatgatggcaggtctacttattcactttttagcttttgacaagaaagcttacttttatcatagcagctttcaaatcctaaacgttgaatacactgaggctttaaactcaccagctacacacgaatacagaaccttgagtgaaagaattgaggctatgattactgatgaatttcgaggatcaagtctaaaaagtgagtttatcaggacacatgttgtcaaactaagaaaagaagggactggtgtggttgcggatgttgtcatgaaatttcgatctagtaaacgtaacaacagaaaggtaatgaaaaccagaattcaatctgtgctacgaagactcagcagctctggaaacttggaaatagccccttcgaatgagataacatcactcactgaccaggatacagaaaatgttttgactcaagaatgtggagcacgtccagaccttataacactgtcagaagagagaatcattggaggcatgcaagctgagcccggtgactggccctggcaagtcagtctacagctcaataatgtccaccactgtggaggtgccctgatcagtaacatgtgggtcctgacagcagctcattgcttcaaaagctatcctaatcctcaatattggacagccacctttggggtttctacaatgagccctaggctgagagtgagagtaagggctattttagcccacgacgggtacagctccgtaactcgtgacaatgacatcgcagttgtacaacttgacagatctgtcgccttttccagaaatatccatagggtatgtctcccagcagcaacccaaaatatcatccctggttctgtcgcatatgttacaggatggggatctctcacatatggaggcaacgcagtcacaaatctacggcaaggagaagtcagaataataagttcagaagaatgcaatacgccagctggttacagtggaagtgtcttgccaggaatgctgtgtgctggaatgcgttcaggggccgtggatgcatgccagggtgattcaggtggcccgctagtacaagaagactcaaggcggctttggtttgttgtgggcattgtgagctggggatatcagtgtggcctcccaaataagccaggcgtgtatactcgagtgacagcctaccgcaactggatcagacagcagacgggaatctagtgcaaccgaggaaaaaacgtgccatgaggtctctgtatccaagtgtgactgactcggatgccatggcttcacatttcaactgcaaaggagactggaaatgccccttctgacgtcccattacataaattggtttaactgtttagt

SEQ ID NO: 5 - RNA, 1462 nt, Mus musculus
cgccgggcaggucaaagcagcuggacacacagaaacaaggaccucuucauuauucaagaguaaaauguauaggccaagaccaaugcuaucaccgucaagauucuucacucccuuugcaguagcuuucguugucauaauaacgguagggcuccuggccaugauggcaggucuacuuauucacuuuuuagcuuuugacaagaaagcuuacuuuuaucauagcagcuuucaaauccuaaacguugaauacacugaggcuuuaaacucaccagcuacacacgaauacagaaccuugagugaaagaauugaggcuaugauuacugaugaauuucgaggaucaagucuaaaaagugaguuuaucaggacacauguugucaaacuaagaaaagaagggacuggugugguugcggauguugucaugaaauuucgaucuaguaaacguaacaacagaaagguaaugaaaaccagaauucaaucugugcuacgaagacucagcagcucuggaaacuuggaaauagccccuucgaaugagauaacaucacucacugaccaggauacagaaaauguuuugacucaagaauguggagcacguccagaccuuauaacacugucagaagagagaaucauuggaggcaugcaagcugagcccggugacuggcccuggcaagucagucuacagcucaauaauguccaccacuguggaggugcccugaucaguaacauguggguccugacagcagcucauugcuucaaaagcuauccuaauccucaauauuggacagccaccuuugggguuucuacaaugagcccuaggcugagagugagaguaagggcuauuuuagcccacgacggguacagcuccguaacucgugacaaugacaucgcaguuguacaacuugacagaucugucgccuuuuccagaaauauccauaggguaugucucccagcagcaacccaaaauaucaucccugguucugucgcauauguuacaggauggggaucucucacauauggaggcaacgcagucacaaaucuacggcaaggagaagucagaauaauaaguucagaagaaugcaauacgccagcugguuacaguggaagugucuugccaggaaugcugugugcuggaaugcguucaggggccguggaugcaugccagggugauucagguggcccgcuaguacaagaagacucaaggcggcuuugguuuguugugggcauugugagcuggggauaucaguguggccucccaaauaagccaggcguguauacucgagugacagccuaccgcaacuggaucagacagcagacgggaaucuagugcaaccgaggaaaaaacgugccaugaggucucuguauccaagugugacugacucggaugccauggcuucacauuucaacugcaaaggagacuggaaaugccccuucugacgucccauuacauaaauugguuuaacuguuuagu

SEQ ID NO: 6 - DNA, 1462 bp, Mus musculus
  exon (65)..(1315)
cgccgggcaggtcaaagcagctggacacacagaaacaaggacctcttcattattcaagagtaaaatgtataggccaagaccaatgctatcaccgtcaagattcttcactccctttgcagtagctttcgttgtcataataacggtagggctcctggccatgatggcaggtctacttattcactttttagcttttgacaagaaagcttacttttatcatagcagctttcaaatcctaaacgttgaatacactgaggctttaaactcaccagctacacacgaatacagaaccttgagtgaaagaattgaggctatgattactgatgaatttcgaggatcaagtctaaaaagtgagtttatcaggacacatgttgtcaaactaagaaaagaagggactggtgtggttgcggatgttgtcatgaaatttcgatctagtaaacgtaacaacagaaaggtaatgaaaaccagaattcaatctgtgctacgaagactcagcagctctggaaacttggaaatagccccttcgaatgagataacatcactcactgaccaggatacagaaaatgttttgactcaagaatgtggagcacgtccagaccttataacactgtcagaagagagaatcattggaggcatgcaagctgagcccggtgactggccctggcaagtcagtctacagctcaataatgtccaccactgtggaggtgccctgatcagtaacatgtgggtcctgacagcagctcattgcttcaaaagctatcctaatcctcaatattggacagccacctttggggtttctacaatgagccctaggctgagagtgagagtaagggctattttagcccacgacgggtacagctccgtaactcgtgacaatgacatcgcagttgtacaacttgacagatctgtcgccttttccagaaatatccatagggtatgtctcccagcagcaacccaaaatatcatccctggttctgtcgcatatgttacaggatggggatctctcacatatggaggcaacgcagtcacaaatctacggcaaggagaggtcagaataataagttcagaggaatgcaatacgccagctggttacagtggaagtgtcttgccaggaatgctgtgtgctggaatgcgttcaggggccgtggatgcatgccagggtgattcaggtggcccgctagtacaagaagactcaaggcggctttggtttgttgtgggcattgtgagctggggatatcagtgtggcctcccaaataagccaggcgtgtatactcgagtgacagcctaccgcaactggatcagacagcagacgggaatctagtgcaaccgaggaaaaaacgtgccatgaggtctctgtatccaagtgtgactgactcggatgccatggcttcacatttcaactgcaaaggagactggaaatgccccttctgacgtcccattacataaattggtttaactgtttagt

SEQ ID NO: 7 - DNA, 28 bp, Artificial Sequence
  PCR primer
aaggatccatggggccgcggcggctgct

SEQ ID NO: 8 - DNA, 28 bp, Artificial Sequence
  PCR primer
tgggaattcctaagttaacagctttttg

SEQ ID NO: 9 - DNA, 29 bp, Artificial Sequence
  PCR primer
cctttggatccaggaggatgcggagcccc

SEQ ID NO: 10 - DNA, 33 bp, Artificial Sequence
  PCR primer
ctgtggaattcccatctgaggacctggaaaact

SEQ ID NO: 11 - DNA, 33 bp, Artificial Sequence
  PCR primer
cctttggatccatgaaagccctcatctttgcag

SEQ ID NO: 12 - DNA, 35 bp, Artificial Sequence
  PCR primer
cctttgaattcctattttgtaaggtaagcagtgga

SEQ ID NO: 13 - DNA, 32 bp, Artificial Sequence
  PCR primer
cctttagatctatgtgggggcgactgctcctg

SEQ ID NO: 14 - DNA, 31 bp, Artificial Sequence
  PCR primer
cctttgaattctgtcactggagcaaagagga

SEQ ID NO: 15 - DNA, 24 bp, Artificial Sequence
  PCR primer
tttactgttttcgtaacagttttg

SEQ ID NO: 16 - DNA, 20 bp, Artificial Sequence
  PCR primer
caacaacgcacagaatctag

SEQ ID NO: 17 - DNA, 20 bp, Artificial Sequence
  PCR primer
ctgacngcngcncaytgctt

SEQ ID NO: 18 - DNA, 18 bp, Artificial Sequence
  PCR primer
gtcrccctgrcangcrtc

SEQ ID NO: 19 - DNA, 20 bp, Artificial Sequence
  PCR primer
acgcattccagcacacagca

SEQ ID NO: 20 - DNA, 27 bp, Artificial Sequence
  PCR primer
cacaaatctacggcaaggagaggtcag

SEQ ID NO: 21 - DNA, 27 bp, Artificial Sequence
  PCR primer
gaacttattattctgacctctccttgc

SEQ ID NO: 22 - DNA, 20 bp, Artificial Sequence
  PCR primer
ggaatgctgtgtgctggaat

SEQ ID NO: 23 - DNA, 41 bp, Artificial Sequence
  PCR primer
ccaaaggatccaaatgtataggccaagaccaatgctatcac

SEQ ID NO: 24 - DNA, 39 bp, Artificial Sequence
  PCR primer
cctttgaattcggaacgtcagaaggggcatttccagtct

SEQ ID NO: 25 - protein, 20 aa, Homo sapiens
ILGGTEAEEGSWPWQVSLRC

SEQ ID NO: 26 - DNA, 1555 bp, Sus scrofa
  CDS (106)..(1359)
  misc_feature (106)..(663): propeptide portion
gttctttctagatccacataatacattagtagtcattcatttgagtggaaatctcagagtggtcagatagcagcaaaaaggacagcctcacaattcaggattaaaatgtataggccagcaacagtacgatcagcttcaagatctctgaatccatacacagtatgttttattgttgtcgcagtggtggtgatcctggcagtgaccatagctctacttgtccactttttagcttttgatcaaaaagcttacttttaccatagtagatttcaaatcctaaatgttgaatatagtgatgagctaaattcaccagctacacagaaatataggtctttgagtggaagaattgaatctatgattactagaacatttaaggagtcaaatttaagaaatcagttcgtgagagctcatgttgtcaaactgaggcaaagtggtagtggtgtgatagcagatattgtcatgaaatttaaattcaccagatataacaatggagcatcaatgaaaagcagaattgagtctgttttacgccaaatgctgaataacactggaaacttggtaatgaacccttcaactgagttaacaccaataacagaccaggatacagtaaatattttcactcaaggatgtggggcccgtccagacctaataacattgtctgaagagagaatcataggcggcactaaggctgaggaaggagactggccctggcaagtcagtctgcagcggaataatcttcatcactgtggaggcgtcttgatcagtaacagatggatcctgactgcagctcactgcttcagaagctactctgatcctcgccagtggactgtcacctttggtatttccactatatttcctaaagacagaataggagtaaggaatattttaatccataacaattataaccctgaaactcatgaaaatgatattgcgcttgtacaactcaacagagaagtcgcctttaccaaaaatatccattcagtgtgtctcccagaggccacccaaactattccacctggttccacggcttatgtaacaggatggggatcacaaagatatagcggcaacacagttccagatctagagcaagtacgggtcaatataataagtaacgatgtatgtaattcgccagctggttataatggggatgtcctgcctggaatgctatgtgctgggctacctgaagggggagcagatgcatgccagggtgactctggtggcccactacagcaggaggactcacggcggctttggttccttgtggggatagtaagctgggggtatcagtgtggtctgccagataagccaggagtgtacactcgagtgacagcctaccgggactggatagcccaacaaactgggatctagcacaataaatacacctctctagcaagccggtgcacacacatgctcgtctacaattccaaactttactttccagccacaaaagaatacatgtttcacaaacactatttaatctttattactatggattttatattctctcaagaagatttagatgaatgttgcatggtactgtggatatatgccgggggaaaca

SEQ ID NO: 27 - protein, 418 aa, Sus scrofa
MYRPATVRSASRSLNPYTVCFIVVAVVVILAVTIALLVHFLAFDQKAYFYHSRFQILNVEYSDELNSPATQKYRSLSGRIESMITRTFKESNLRNQFVRAHVVKLRQSGSGVIADIVMKFKFTRYNNGASMKSRIESVLRQMLNNTGNLVMNPSTELTPITDQDTVNIFTQGCGARPDLITLSEERIIGGTKAEEGDWPWQVSLQRNNLHHCGGVLISNRWILTAAHCFRSYSDPRQWTVTFGISTIFPKDRIGVRNILIHNNYNPETHENDIALVQLNREVAFTKNIHSVCLPEATQTIPPGSTAYVTGWGSQRYSGNTVPDLEQVRVNIISNDVCNSPAGYNGDVLPGMLCAGLPEGGADACQGDSGGPLQQEDSRRLWFLVGIVSWGYQCGLPDKPGVYTRVTAYRDWIAQQTGI

SEQ ID NO: 28 - DNA, 1520 bp, Macaca fascicularis
  CDS (100)..(1356)
  misc_feature (100)..(657): propeptide portion
aactatagatccacgtaatacatgagagtcattcatttgagtgggaatcycaaagcagttgagtaggcagaaaaaggagcctcttcattaaggattaaaatgtataggccagcacgtgtaccatcgacttcaagatttctgaatccatatgtcgtatgtttcattgtcgtcgcaggggtagtgatcctggcagtgaccatagctctacttgtttactttttagcttttgatcaaaaatcttacttttacaggagcagttttcaactcctaaatgttgaatataatagtcagttaaattcaccagctacacaggaatacaggactttgagtggaagaattgaatctctgattactagaacattcaaagaatcaaatttaagaaaccagttcatgagagctcatgttgtcaaactgagtcaagatggtagtggtgtgagagcggatgttgtcatgaaatttcgattcactagaaataacaatggagcatcaatgaaaagcagaattgagtctgttttacaacaaatgctgaataactctggaaatttggaaataaactcttcaactgagataacatcacttactgaccaggctgcagcaaattggcttattaaygaatgtggggccggtccagacctaataacattgtctgagcagagaatcattggaggcactgaggctgaggagggaagctggccatggcaagtcagtctacgggtaaataatgcccaccactgtggaggcagcctgatcagtaacacgtggatcctgacagcagctcactgcttcagaagcaactccaatcctcgtgaatggattgccacctttggtatttccacaacaaatcctagactaagaatgagagtaagaagtattttaattcataacaattatatatctgcaactcatgaaaatgacattgcacttgtgagacttgagaacagtgtcacctttaccagagacatccatagtgtgtgtctcccagctgctacccagaatattccacttggctctactgcttatgtaacaggatggggtgctcaagaatatgccggctccacagtttcagagctaaggcaagcacaagtcagaataataagtaatgatgtatgtaatgcaccatatagttataatggaggcatcttgcccggaatgctatgtgctggagtacctcaaggtggagtggatgcatgtcagggtgactctggtggccccctagtacaggaagactcacggcggctttggtttcttgtggggatagtaagctggggagatcagtgtggcctgccagataggccaggagtgtatacccgagtgacaacctaccgagactggattaggcaacgaactgggatctagtgcaccaagtgcatccctgttgcaaagtctgtatgcgggtgtgcctgtcttaaattccaaagctttacatttcaaccgaaaaagaaactggaaatgtcctaatttaacaacttgttacataaacatggtttaataataataaaaaaaaaaaaaaaaaaaaaaaa

SEQ ID NO: 29 - protein, 418 aa, Macaca fascicularis
MYRPARVPSTSRFLNPYVVCFIVVAGVVILAVTIALLVYFLAFDQKSYFYRSSFQLLNVEYNSQLNSPATQEYRTLSGRIESLITRTFKESNLRNQFMRAHVVKLSQDGSGVRADVVMKFRFTRNNNGASMKSRIESVLQQMLNNSGNLEINSSTEITSLTDQAAANWLINECGAGPDLITLSEQRIIGGTEAEEGSWPWQVSLRVNNAHHCGGSLISNTWILTAAHCFRSNSNPREWIATFGISTTNPRLRMRVRSILIHNNYISATHENDIALVRLENSVTFTRDIHSVCLPAATQNIPLGSTAYVTGWGAQEYAGSTVSELRQAQVRIISNDVCNAPYSYNGGILPGMLCAGVPQGGVDACQGDSGGPLVQEDSRRLWFLVGIVSWGDQCGLPDRPGVYTRVTTYRDWIRQRTGI

SEQ ID NO: 30 - DNA, 1406 bp, Canis familiaris
  CDS (110)..(1366)
  misc_feature (110)..(667): propeptide portion
gagttccttcatagatcacacataatatatgagagtcattcatttgtgtagaaatctcagagaagttagataggcagcagaaaggatctcttcattattcaggatcaaaatgtataggccagcacgggtgccatcaggctcaagattcctgaatccatatgtagtgtgtttcgttgttgtggcaggggtggtaatcctggccgtaaccatagctctactcatccacttcttagcgtttgatcaaaagtcttacttttaccatagcagttttcaaatcctaaatgtccaatatagtaatcaattaaattcaccagggacacaagaatacaggactttgagtggaagaattgaatctctgattactaaaacattcagagaatcaaatttaagaaatcagttcatcagagctcatgttgtcaaactgaggcaagaaggtaatggtgtgatagcagatgttgtcatgaaatttaaattcactagaaataacaatggagcattgatgaaaagcagaattaagtctgttttacaccaaatgctgaataattctggaaacttggaaataagcccttcaactgagataacatccattactgaccaggatacagtaaatattttcactgaaggatgtggggcccgtccagacctaataactttgtctgaggagaggatcctaggaggcaacaaggctgaagaaggagattggccatggcaagtcagtctacagaagaataatgttcaccactgtggaggtgtcctgatcagtagcatgtggatcctgtcagcagctcactgcttcagaagccaatctaatcctcgtcagtggactgccatctttggtgcttcaatagcatttcctaaacagaaaagaagagtaaggactattttaatccataacaattataaccctgcaactcatgaaaatgatattgcagctatacaacttgaaggaggtatcaactttaccaaaaatatccatagggtgtgtctcccagaggctacccagaatattccacctggttcttctgcttatgtaacaggatggggatctcaagaatacggtggtaacacagtttcagatctacagcaagcacgggtcagaataataagtaatgatgtatgtaatgcaccaactagttataacggagctgtcaggcctggaatgctctgtgctggcctacctcaaggtggagtggatgcatgccggggtgactcgggtggcccactggttcaagaggactcacggcggctttggttcctcgtgggaatagtaagctggggggaccgatgcggtctgccagataagccaggagtgtacactcgagtgacagcctaccgtgactggataactgaaaaaactggagtctagcacaataaatgcatctttgtgaaaaaaaaaaaaaaaaaaa

SEQ ID NO: 31 - protein, 418 aa, Canis familiaris
MYRPARVPSGSRFLNPYVVCFVVVAGVVILAVTIALLIHFLAFDQKSYFYHSSFQILNVQYSNQLNSPGTQEYRTLSGRIESLITKTFRESNLRNQFIRAHVVKLRQEGNGVIADVVMKFKFTRNNNGALMKSRIKSVLHQMLNNSGNLEISPSTEITSITDQDTVNIFTEGCGARPDLITLSEERILGGNKAEEGDWPWQVSLQKNNVHHCGGVLISSMWILSAAHCFRSQSNPRQWTAIFGASIAFPKQKRRVRTILIHNNYNPATHENDIAAIQLEGGINFTKNIHRVCLPEATQNIPPGSSAYVTGWGSQEYGGNTVSDLQQARVRIISNDVCNAPTSYNGAVRPGMLCAGLPQGGVDACRGDSGGPLVQEDSRRLWFLVGIVSWGDRCGLPDKPGVYTRVTAYRDWITEKTGV

SEQ ID NO: 32 - DNA, 1381 bp, Bos taurus
  CDS (73)..(1326)
  misc_feature (73)..(627): propeptide portion
gggattcatttgagtggaaatctcagagcagttgcataggcggaaaaaaggacttcattattaaggattaaaatgtataggccagcacgagtgacatcagcctcaagatccctgaatccatatctagtattttttgttgtcgtcgcagtggtggtgatcctggcagtgatcataggtctacttgtctactttttagcttttgatcaaaaatcttacttttaccaaagcagcattcaaatcttgggtaaacaatatagtgatgagttaagttcaccagctacagagaaatataggactttgagtggaagaattgaatctatgattactaaaacattcaaagagtcagatttaaaaaatgagttcatcaaagctcatgttgtcaaactgaggcaaagtggcaataatgtgatagcagatattatcatgaaatttaaattcaccagaagaatcaatgaagcatcaatgaaaagcagaattgagtctattttacgccaaatgccgaataactctgaagacttgaacatgaatccaactcaggtaatatcaataactggccaggatacaatacaactgttcactcgagaatgtggggtccgctcagacctgataaccttgtctgaggagagaatcataggaggcagtaaagctgagaaaggagactggccatggcaagtcagtctacagtggagcagttctcaccgctgcggaggagccttgatcagtaataggtggatcctgtcagcagctcactgcttcagaagccactctgatcctcgccaatggattgccacctttggtacttccacaatatctcctcaactgagagtaggagtaaggaatattttaatccatgacaattataaacctgaaactcatgaaaacgatattgcactcgtacaacttgatagagaagtcacctttaacagatatattcatacagtgtgtctcccagaggctaaccaggccatttcagctggttccactgcttatgtaacaggatggggatctcagagttatagcggcagcacagtttcagatctaaaccaaggacgggtcaatataataagtaatactgtatgtaacacaccagctggttataatggagccgtcctgtctggaatgctatgtgctggactacctgaaggtggagtggacgcgtgccagggtgactctggtggccctctagtacaagaagactcacggcaacactggttcatcgtggggatagtaagctgggggtatcaatgtggtctgccagataaaccaggagtatacactagagtgacagcctaccgtgactggataacccaacaaactggtatctagtgcaataaatacatcttggttcaagagccaaaaaaaaaaaaaaaaaaaaaaaaaa

SEQ ID NO: 33 - protein, 417 aa, Bos taurus
MYRPARVTSASRSLNPYLVFFVVVAVVVILAVIIGLLVYFLAFDQKSYFYQSSIQILGKQYSDELSSPATEKYRTLSGRIESMITKTFKESDLKNEFIKAHVVKLRQSGNNVIADIIMKFKFTRRINEASMKSRIESILRQMPNNSEDLNMNPTQVISITGQDTIQLFTRECGVRSDLITLSEERIIGGSKAEKGDWPWQVSLQWSSSHRCGGALISNRWILSAAHCFRSHSDPRQWIATFGTSTISPQLRVGVRNILIHDNYKPETHENDIALVQLDREVTFNRYIHTVCLPEANQAISAGSTAYVTGWGSQSYSGSTVSDLNQGRVNIISNTVCNTPAGYNGAVLSGMLCAGLPEGGVDACQGDSGGPLVQEDSRQHWFIVGIVSWGYQCGLPDKPGVYTRVTAYRDWITQQTGI

SEQ ID NO: 34 - DNA, 1419 bp, Oryctolagus cuniculus
  CDS (73)..(1329)
  misc_feature (73)..(630): propeptide portion
gtcacttgagtgggaatctcaaagcagttggattggcagaaaaataggacctcttcgctactcaggattaaaatgtataggccagcacggggatcatcgacttcaagattcctgaatccatacgtgatttgtttcattgttgtagcagtcgtggtgatcctggcagtgatcgtagctctacttattcactttttagcttttgataaaaagtcttgcttttttcacagcagctttcaaattcgaaatgttcaatatagcgatcagttaaattcaccagctacacaggaatacagattattgagtgaaagaattgaatctatgattagtcaaacactacaaggatcaaacttaagaaatcagttcattagagctcatgttgtcaaactgaggcaggatagtaatagtgtgatagcagatgttgtaatgaaattccgagtcagtagaaacaacaatggtgatgcaatgaaaagaagggttcaggatgttttacagcaaatgctgaataactctggaagcttggaaataaacccttcaactacggtaacagagattactggccaggatacagaaactatcttcacccaacaatgtggggcccgtccagacctaataacattgtctgaagagagaatcattggaggcacccaggctgaggagggagactggccctggcaagtcagtctccagcccaacaatgctcaccattgtggaggcattttgatcagtaacacgtggatcctgacagcagcacactgcttccggagctactctgatcctcgtcaatggactgccacctttggtatttccacagcagtttctagacagagaatgagaataaggacaattttagtccataacaattatagatctgcaacgcatgaaaatgatattgcagctgtgcaacttgaaggagctatcacatttacaagaaacatccatagtgtgtgtctcccagaggccactcagaacattacacctggttcttcagcttatgtaacaggatgggggtctctagaatatggtggcaacacggttacctatctacagcaaggacgggtcagaataataagtaatgaagtatgtaacgcaccagcgagttacaatggcgctgtcttgcctacaatggtgtgtgcaggattatctcaaggaggagtggacgcatgccagggtgactctggtggcccactggtacaagaagactcgcgccggctttggttcgttgtgggcattgtaagctggggctatcagtgtggcctgcctgacaaaccaggagtgtacacacgagtgacagcctaccgtgactggattagagaacaaacggggatctagctggataaatgcacctctgctgcaaagccggttcgcaactgagcctgtcaaaaatccaagctttaattctccgctgaaaagaacaggaag

SEQ ID NO: 35 - protein, 418 aa, Oryctolagus cuniculus
MYRPARGSSTSRFLNPYVICFIVVAVVVILAVIVALLIHFLAFDKKSCFFHSSFQIRNVQYSDQLNSPATQEYRLLSERIESMISQTLQGSNLRNQFIRAHVVKLRQDSNSVIADVVMKFRVSRNNNGDAMKRRVQDVLQQMLNNSGSLEINPSTTVTEITGQDTETIFTQQCGARPDLITLSEERIIGGTQAEEGDWPWQVSLQPNNAHHCGGILISNTWILTAAHCFRSYSDPRQWTATFGISTAVSRQRMRIRTILVHNNYRSATHENDIAAVQLEGAITFTRNIHSVCLPEATQNITPGSSAYVTGWGSLEYGGNTVTYLQQGRVRIISNEVCNAPASYNGAVLPTMVCAGLSQGGVDACQGDSGGPLVQEDSRRLWFVVGIVSWGYQCGLPDKPGVYTRVTAYRDWIREQTGI

SEQ ID NO: 36 - DNA, 1576 bp, Cavia porcellus
  CDS (102)..(1358)
  misc_feature (102)..(659): propeptide portion
ggacttctgctcctcagacactcagcactccagtcatttcagtgggaatctcaaaggagttgacaagcaggagaacgatcgcttcatctttcaggataaagatgtataggccagcaacagtgtcatctaactcgagattcctgaacccctgggtagtcttcttcactgtgttcgcggtggtggggatcctggccatgattgtaggactgctcattcactttttagcttttgatcaaaaatcttgcttttaccacagtgatgttcaaataatgaatgttgaatacaatgatcagttaagctcacccggtacacaagaatacaggattttgagtgaaaggattgaatctatgatcactaatgcattccagcagtccaatttaagaaatcagtttatcagagcccatgttgtaagactgaggcaagagggtaacggtgtggtagcagatgttgtcatgaagtttcgattcagtagacgtaacaatggagaatccatgaaagccagaattcagtctattttacagcaaatgctgaataactctggaaacctggaaataagcccttcagccggggtaacagaaattaacgaccaggaaacagaaaatatgtttactcaagcatgcggggcccgtccagacctgatgacgctgtctgcagagagagtcgttggaggtactcaagctgaccagggcgactggccgtggcaagtcagtctgcaggtcaacggtggccatcgctgcggaggcgtcctggtcagcaaccagtgggtcctgactgcagcccactgcttcagaagctaccctaatgctcaacaatggactgccacctttggtatttccacaacctctcctactctgagagtgagagtgaggactatttcaatccacaacaattacaatcctgtgactcatgagaatgatattgcagctgtgcagctggaaagggctgtcaccttcaccagggatgttcaccgagtatgtctccccgcagccacccagactgtcacacctggttctacagcttatgtaacaggatggggctcgataatctatggtggcaacacggtcagatatctacggcaaggacaagtccagataataagtactagtgagtgtaacgcaccagccagttacaacggtgctgtcctgcctgggatgctgtgtgctggcgtgccgacaggtgcagtggacgcgtgccagggagattctggtggcccactagtccaagaagactcacggcggctttggttcctggtgggaatagtgagctggggctatcagtgtggtgtgcccgacaagcccggagtatatactcgagtgaccacataccgtaactggattagacaagtaactggggtctagcgcaacccatgcatttctgttgcaaagtgatcaagtgcaataaatgcatctctgttgcaaagtctgaatgcaggtgtgcctggctacaattccaaagctttacttttcagcagaaaaataaagctgcgcttgtttcatttttacatactgttacaaaatagagcagaataaaacattattcactcttcctcaaaaaaaaaaaaaaaaaaaaaaaaaaa

SEQ ID NO: 37 - protein, 418 aa, Cavia porcellus
MYRPATVSSNSRFLNPWVVFFTVFAVVGILAMIVGLLIHFLAFDQKSCFYHSDVQIMNVEYNDQLSSPGTQEYRILSERIESMITNAFQQSNLRNQFIRAHVVRLRQEGNGVVADVVMKFRFSRRNNGESMKARIQSILQQMLNNSGNLEISPSAGVTEINDQETENMFTQACGARPDLMTLSAERVVGGTQADQGDWPWQVSLQVNGGHRCGGVLVSNQWVLTAAHCFRSYPNAQQWTATFGISTTSPTLRVRVRTISIHNNYNPVTHENDIAAVQLERAVTFTRDVHRVCLPAATQTVTPGSTAYVTGWGSIIYGGNTVRYLRQGQVQIISTSECNAPASYNGAVLPGMLCAGVPTGAVDACQGDSGGPLVQEDSRRLWFLVGIVSWGYQCGVPDKPGVYTRVTTYRNWIRQVTGV

SEQ ID NO: 38 - DNA, 1538 bp, Mesocricetus auratus
  CDS (206)..(1459)
  misc_feature (206)..(760): propeptide portion
gtcccagtctctaattctggtcccaagtacagtgttggttcaagttcagtctttcagacttgttttgcaagttaggttttaattacatgggatttcctcctttggcttcagatacacatagcacaaaagagtcactcaattgagtgggaatctcaaagcatctggacacacagaagaaaggacctcgccattactcaggagtaaaatgtataggccaagaccagtgataccacaatcaaggttcttcagtccgtttgtagtggcttttgttgtcataacaacrgtactgatcctggccatgactataggtctacttattcacttcttagcttttgacaagaaaacttacttttaccacagcagctttcaaatcctaaatgttgaatacactgaggctttaaattccccagctacacaggaatacaggcatttgagtgaaagaattgaatctatgattactgatgcattccgagaatcaaatttaagaagtgagtttatcagaacacacgttgttaaactgaggaaagaagggaatggtgtgatcgcagatgctgtcatgaaatttcgatctagtaaacgcagcaatagaaaatccatgaaaaacagaattcattctgtgctacaaatactgagtaactctggaagtttggaaataaccccttcaaatgagataacatcactcactgaccaagatacagaaaattttttgactcaagaatgtggagcccgtccagaccttataacactgtcagaagagagagttattggaggcactctagctgaaacaggtgactggccctggcaagtcagtctacaactcaataatgtccaccactgtggaggtatcctgatcagtaacttgtgggtcctgacagcagctcactgcttcagaagctactctaatcctctacaatggactgccacctttggtgtttctacaatacgtcctatcttaagagtaagagtaaggtctattgtatcccataacaattacagacctacaactcgtgataatgatattgcagttgtacaacttgaaagacctatcacctttaacagaaatatccacagggtgtgtctcccagctgcgacccaaagtatcatacctggttctattgcatatgtcacaggatgggggtcgctcacttatggaggcaacacagtcaccgatctacggcaaggacaggtcagaatagtaagtaccgacgagtgcaatgaaccagctgggtacagtggaagcgtcttgcctggaatgctctgtgctggagtgccttcaggtgctgtggatgcgtgccaaggtgattctggtggcccactagtacaggaagactcacggatgctttggtttgttgtggggattgtaagctggggatatcagtgcggcctgccaaataaaccaggagtgtacacgcgagtgacaacctaccgcgactggattcgccagcaagctggagtctagtgccacgaaagtgctgtgaagtttgtgtgcaaatgtcctgactcagattcaactgcaaaagaaaccggaatgtctattt

SEQ ID NO: 39 - protein, 417 aa, Mesocricetus auratus
  misc_feature (26)..(26): The ′Xaa′ at location 26 stands for Thr.
MYRPRPVIPQSRFFSPFVVAFVVITXVLILAMTIGLLIHFLAFDKKTYFYHSSFQILNVEYTEALNSPATQEYRHLSERIESMITDAFRESNLRSEFIRTHVVKLRKEGNGVIADAVMKFRSSKRSNRKSMKNRIHSVLQILSNSGSLEITPSNEITSLTDQDTENFLTQECGARPDLITLSEERVIGGTLAETGDWPWQVSLQLNNVHHCGGILISNLWVLTAAHCFRSYSNPLQWTATFGVSTIRPILRVRVRSIVSHNNYRPTTRDNDIAVVQLERPITFNRNIHRVCLPAATQSIIPGSIAYVTGWGSLTYGGNTVTDLRQGQVRIVSTDECNEPAGYSGSVLPGMLCAGVPSGAVDACQGDSGGPLVQEDSRMLWFVVGIVSWGYQCGLPNKPGVYTRVTTYRDWIRQQAGV

SEQ ID NO: 40 - DNA, 21 bp, Artificial Sequence
  PCR primer
ctggccstggcaagtcagtct

SEQ ID NO: 41 - DNA, 29 bp, Artificial Sequence
  PCR primer
gtggrtcctgacwgcmgcycaytgcttca

SEQ ID NO: 42 - DNA, 27 bp, Artificial Sequence
  PCR primer
tgwgtcttcytgkactagygggccacc

SEQ ID NO: 43 - DNA, 29 bp, Artificial Sequence
  PCR primer
caytgatmkccccagctyacwatbcccac

SEQ ID NO: 44 - DNA, 1998 bp, Artificial Sequence
  fused DNA for human EGF and human placental alkalyne phosphatase
atgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccatgtcttcggaaatacaaggacttctgcatccatggagaatgcaaatatgtgaaggagctccgggctccctcctgcatctgccacccgggttaccatggagagaggtgtcatgggctgagcctcccagtggaaaatcgcttatatacctatgaccacacaaccatcctgatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatga

SEQ ID NO: 45 - protein, 8 aa, Artificial Sequence
  peptide fragment
NSGNLEIN

SEQ ID NO: 46 - protein, 8 aa, Artificial Sequence
  peptide fragment
ILGGTEAE

SEQ ID NO: 47 - protein, 8 aa, Artificial Sequence
  peptide fragment
DQAAANWL

SEQ ID NO: 48 - protein, 4 aa, Artificial Sequence
  peptide fragment
ILGG

SEQ ID NO: 49 - protein, 7 aa, Artificial Sequence
  peptide fragment
INEGAGP

SEQ ID NO: 50 - DNA, 38 bp, Artificial Sequence
  PCR primer
cccagatctgaattcagtaacccaggcattattttatc

SEQ ID NO: 51 - DNA, 30 bp, Artificial Sequence
  PCR primer
aactttggatccactccgtatttgataagg

SEQ ID NO: 52 - DNA, 32 bp, Artificial Sequence
  PCR primer
catgtttacacacagtgagaatggttccttcc

SEQ ID NO: 53 - DNA, 35 bp, Artificial Sequence
  PCR primer
ctgtgtgtaaacatggaagacgccaaaaacataaa

SEQ ID NO: 54 - DNA, 20 bp, Artificial Sequence
  PCR primer
cgggaggtagatgagatgtg

SEQ ID NO: 55 - DNA, 31 bp, Artificial Sequence
  PCR primer
gacttcgcgacgtgaggctccggtgcccgtc

SEQ ID NO: 56 - DNA, 38 bp, Artificial Sequence
  PCR primer
gactggtaccaagcttttcacgacacctgaaatggaag

SEQ ID NO: 57 - DNA, 22 bp, Artificial Sequence
  PCR primer
tcaacggagactgcgagtacac

SEQ ID NO: 58 - DNA, 19 bp, Artificial Sequence
  PCR primer
tcttgatggccttggagca

SEQ ID NO: 59 - DNA, 29 bp, Artificial Sequence
  probe
actcctttcgtgttgtcaccgagaacgtc